(19) United States
(12) Patent Application Publication
(10) Pub. No.: US 2009/0018166 A1
(43) Pub. Date: Jan. 15, 2009

(54) New Pyridine Analogues X 161

(75) Inventors: Kosrat Amin, Molndal (SE); Thomas Antonsson, Molndal (SE); Christoffer Bengtsson, Molndal (SE); David Brown, Molndal (SE); Ruth Bylund, Molndal (SE); Daniel Hovdal, Molndal (SE); Fabrizio Giordanetto, Molndal (SE); Johan Johansson, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/972,787

(22) Filed: Jan. 11, 2008

Related U.S. Application Data
(60) Provisional application No. 60/949,687, filed on Jul. 13, 2007.

(51) Int. Cl. A61K 31/4545; C07D 401/04

(52) U.S. Cl. 514/318; 546/193; 546/208

(57) ABSTRACT

The present invention relates to certain new pyridin analogues of Formula (I)

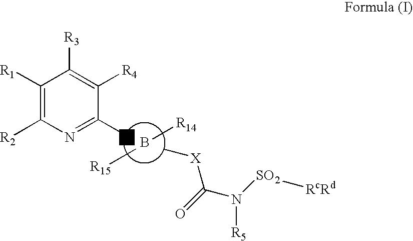

to processes for preparing such compounds, to their utility as P2Y₁₂ inhibitors and as anti-trombotic agents etc, their use as medicaments in cardiovascular diseases as well as pharmaceutical compositions containing them.

### FIELD OF THE INVENTION

The present invention provides novel pyridine compounds, their use as medicaments, compositions containing them and processes for their preparation.

### BACKGROUND OF THE INVENTION

Platelet adhesion and aggregation are initiating events in arterial thrombosis. Although the process of platelet adhesion to the sub-endothelial surface may have an important role to play in the repair of damaged vessel walls, the platelet aggregation that this initiates can precipitate acute thrombotic occlusion of vital vascular beds, leading to events with high morbidity such as myocardial infarction and unstable angina. The success of interventions used to prevent or alleviate these conditions, such as thrombolysis and angioplasty is also compromised by platelet mediated occlusion or re-occlusion.

Haemostasis is controlled via a tight balance between platelet aggregation, coagulation and fibrinolysis. Thrombus formation under pathological conditions, like e.g. arteriosclerotic plaque rupture, is firstly initiated by platelet adhesion, activation and aggregation. This results not only in the formation of a platelet plug but also in the exposure of negatively charged phospholipids on the outer platelet membrane promoting blood coagulation. Inhibition of the build-up of the initial platelet plug would be expected to reduce thrombus formation and reduce the number of cardiovascular events as was demonstrated by the anti-thrombotic effect of e.g. Aspirin (BMJ 1994; 308: 81-106 Antiplatelet Trialists' Collaboration. Collaborative overview of randomised trials of antiplatelet therapy, I: Prevention of death, myocardial infarction, and stroke by prolonged antiplatelet therapy in various categories of patients).

Platelet activation/aggregation can be induced by a variety of different agonists. However, distinct intracellular signalling pathways have to be activated to obtain full platelet aggregation, mediated via G-proteins G_{q}, G_{12/13} and Gᵢ (Platelets, A D Michelson ed., Elsevier Science 2002, ISBN 0-12-493951-1; 197-213: D Woulfe, et al. Signal transduction during the initiation, extension, and perpetuation of platelet plug formation) In platelets, the G-protein coupled receptor P2Y₁₂ (previously also known as the platelet P_{2T}, P2T_{ac}, or P2Y_{cyc} receptor) signals via Gi, resulting in a lowering of intra-cellular cAMP and full aggregation (Nature 2001; 409: 202-207 G Hollopeter, et al. Identification of the platelet ADP receptor targeted by antithrombotic drugs.). Released ADP from dense-granules will positively feedback on the P2Y12 receptor to allow full aggregation. WO 2002/098856 and WO 2004/052366 describe piperazino-carbonylmethylaminocarbonyl-naphtyl or -quinolyl derivatives as ADP receptor antagonist.

Clinical evidence for the key-role of the ADP-P2Y₁₂ feedback mechanism is provided by the clinical use of clopidogrel, an thienopyridine prodrug which active metabolite selectively and irreversibly binds to the P2Y₁₂ receptor, that has shown in several clinical trials to be effective in reducing the risk for cardiovascular events in patients at risk (Lancet 1996; 348: 1329-39: CAPRIE Steering committee, A randomised, blinded, trial of clopidogrel versus aspirin in patients at risk of ischaemic events (CAPRIE); N Engl J Med 2001; 345 (7): 494-502): The Clopidogrel in Unstable Angina to prevent Recurrent Events Trial Investigators. Effects of clopidogrel in addition to aspirin in patients with acute coronary syndromes without ST-segment elevation.). In these studies, the clinical benefit with a reduced bleeding risk as compared to thienopyridines (Sem Thromb Haemostas 2005; 31 (2): 195-204 J J J van Giezen & R G Humphries. Preclinical and clinical studies with selective reversible direct P2Y₁₂ antagonists. WO 2005/000281 describes a serie of pyrazolidine-3,5-dione derivatives and WO 2006/1147742 describes a serie of phenyl-pyrimidine derivatives which both series have been described as P2Y12 antagonists for the potential treatment of thrombosis. Other applications disclosing some P2Y12 antagonists for the potential treatment of thrombosis are WO 2006/073361 and WO 2007/008140.

It is an object of the present invention to provide improved, potent, reversible and selective P2Y₁₂-antagonists as anti-trombotic agents.

### SUMMARY OF THE INVENTION

We have now surprisingly found that certain pyridine compounds of Formula (I) or a pharmaceutically acceptable salt thereof are reversible and selective P2Y₁₂ antagonists, hereinafter referred to as the compounds of the invention. The compounds of the invention, having improved stability towards esterases, unexpectedly exhibit improved beneficial properties that render them particularly suitable for use in the treatment of diseases/conditions as described below (See p. 96-97). Examples of such beneficial properties are high potency, high selectivity, beneficial pharmacokinetic properties and an advantageous therapeutic window.

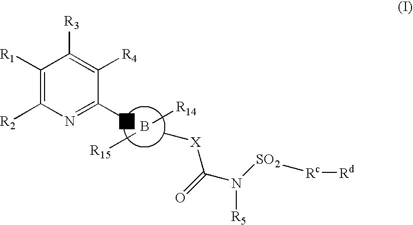

### DETAILED DESCRIPTION OF THE INVENTION

According to the present invention there is provided a novel compound of formula (I) or a pharmaceutically acceptable salt thereof:

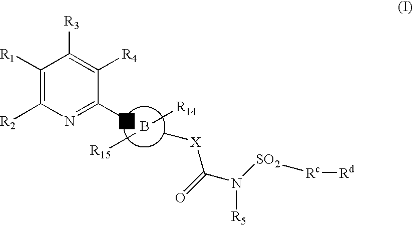

wherein

R₁ represents R₇C(O), R₁₇S, R₁₈C(S) or a group gII

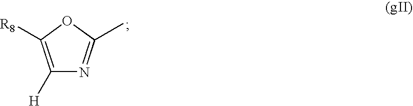

preferably R₁ represents R₇C(O) or the group (gII) below;

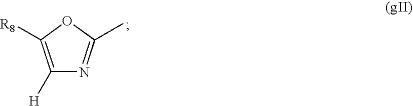

R₂ represents substituted (C₁-C₁₂)alkyl optionally interrupted by sulphur, substituted (C₁-C₁₂)alkoxy or substituted (C₁-C₁₂)alkylthio, wherein any one of these groups is substituted by one or more of azido, carboxy, cyano, (C₁-C₁₂)alkylcarbonyloxy, hydroxy(C₁-C₁₂)alkylcarbonyloxy, arylcarbonyloxy, heterocyclylcarbonyloxy, (C₁-C₁₂)alkyloxycarbonyl, (C₁-C₁₂)alkyl(C(S)), (C₁-C₁₂)alkyl(S(CO)), (C₁-C₁₂)alkylthio, hydroxy(C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl, heterocyclyl(C₁-C₁₂)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₁₂)alkyl, (C₁-C₁₂)alkylcarbonyl or R^{a(2)} and R^{b(2)} together with the nitrogen atom represent piperidine, pyrrolidine, azetidine or aziridine or any one of the groups

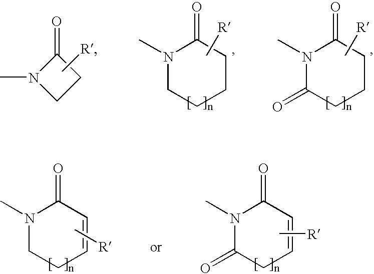

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;

Further R₂ represents substituted (C₁-C₁₂)alkoxy or substituted (C₁-C₁₂)alkylthio, wherein any one of these groups is substituted by one or more of any one of OH, aryl, (C₃-C₆)cycloalkyl or heterocyclyl; Further R₂ represents (C₁-C₁₂)alkylthio, substituted by one or more halogen (F, Cl, I, Br) atom(s); Further R₂ represents (C₁-C₁₂)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₁₂)alkoxy, (C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl and heterocyclyl(C₁-C₁₂)alkylcarbonyl; Further R₂ represents unsubstituted (C₁-C₁₂)alkyl with the proviso that at the same time R₅ represents carboxy(C₁-C₁₂)alkyl; Further R₂ represents a group of formula ((R^{a(2)})N(R^{b(2)}))(CO)—, in which R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₁₂)alkyl optionally interrupted by oxygen and/or optionally substituted by OH, aryl, cycloalkyl, heterocyclyl or one or more halogen (F, Cl, Br, I) atoms; further R₃ represents (C₁-C₁₂)alkoxy optionally substituted by one or more halogen (F, Cl, Br, I) atoms; further R₃ represents (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₁₂)alkyl optionally interrupted by oxygen and/or optionally substituted by OH, COOH, (C₁-C₆)alkoxycarbonyl, aryl, cycloalkyl, heterocyclyl or one or more halogen (F, Cl, Br, I) atoms; further R₄ represents (C₃-C₆)cycloalkyl, hydroxy(C₁-C₁₂)alkyl, (C₁-C₁₂)alkylC(O), (C₁-C₁₂)alkylcycloalkyl, (C₁-C₁₂)alkoxy wherein the alkoxy group may optionally be substituted by one or more halogen (F, Cl, Br, I) atoms, OH and/or COOH and/or (C₁-C₆)alkoxycarbonyl; further R₄ represents (C₁-C₁₂)alkylthioC(O), (C₁-C₁₂)alkylC(S), (C₁-C₁₂)alkoxyC(O), (C₃-C₆)cycloalkoxy, aryl, aryl(C₁-C₁₂)alkoxy, aryl(C₁-C₁₂)alkyl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl, (C₃-C₆)cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;

R₅ represents H or (C₁-C₁₂)alkyl or carboxy(C₁-C₆)alkyl; with the proviso that when R₂ is unsubstituted (C₁-C₁₂)alkyl, R₅ represents carboxy(C₁-C₁₂)alkyl;

R₇ represents (C₁-C₁₂)alkyl optionally interrupted by oxygen, and/or optionally substituted by OH, aryl, cycloalkyl, heterocyclyl or one or more halogen (F, Cl, Br, I) atoms; further R₇ represents (C₂-C₁₂)alkenyl optionally interrupted by oxygen, and/or optionally substituted by OH, aryl, cycloalkyl, heterocyclyl or one or more halogen (F, Cl, Br, I) atoms; further R₇ represents (C₂-C₁₂)alkynyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₁₂)alkyl, aryl or heterocyclyl;

R₈ represents H, (C₁-C₁₂)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;

R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₄ represents aryl, aryl(C₁-C₁₂)alkoxy, aryl(C₁-C₁₂)alkyl, (C₃-C₆)cycloalkyl(C₁-C₁₂)alkoxy, heterocyclyl, a halogen (F, Cl, Br, I) atom, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O), (C₁-C₁₂)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{e}; wherein R^{e} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₅ represents aryl, aryl(C₁-C₁₂)alkoxy, aryl(C₁-C₁₂)alkyl, (C₃-C₆)cycloalkyl(C₁-C₁₂)alkoxy, heterocyclyl, a halogen (F, Cl, Br, I) atom, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a}(15) and R^{b(15)} independently represent H, (C₁-C₁₂)alkyl, (C₁-C₁₂)alkylC(O)), (C₁-C₁₂)alkoxyC(O) or R^{a(15)} and R^{b(15)} together with the nitrogen atom represent piperidine, pyrrolidine, azetidine or aziridine;

R₁₇ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R₁₈ represents (C₁-C₁₂)alkyl optionally interrupted by oxygen and/or optionally substituted by OH, aryl, cycloalkyl, heterocyclyl or one or more halogen (F, Cl, Br, I) atoms; further R₁₈ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R^{c} is a single bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₁₂)alkyl, (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, halogen substituted (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

Preferred values of each variable group or specific embodiments of variable groups or terms are as follows. Such values or embodiments may be used where appropriate with any of the values, definitions, claims, aspects, embodiments or embodiments of the invention defined hereinbefore or hereinafter. In particular, each may be used as an individual limitation on the broadest definition of formula (I).

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the particular definitions for that group.

It will be understood that when formula I compounds contain a chiral centre, the compounds of the invention may exist in, and be isolated in, optically active or racemic form. The invention includes any optically active or racemic form of a compound of formula I which act as P2Y₁₂ receptor antagonists. The synthesis of optically active forms may be carried out by standard techniques of organic chemistry well known in the art, for example by, resolution of a racemic mixture, by chiral chromatography, synthesis from optically active starting materials or by asymmetric synthesis.

It will also be understood that the compounds of the formula I may exhibit the phenomenon of tautomerism, the present invention includes any tautomeric form of a compound of formula I which is a P2Y₁₂ receptor antagonist.

It will also be understood that in so far as compounds of the present invention exist as solvates, and in particular hydrates, these are included as part of the present invention.

It is also to be understood that generic terms such as “alkyl” include both the straight chain and branched chain groups such as butyl and tert-butyl. However, when a specific term such as “butyl” is used, it is specific for the straight chain or “normal” butyl group, branched chain isomers such as “t-butyl” being referred to specifically when intended.

In one embodiment alkyl is, unless otherwise specified, unsubstituted, with the proviso that when R₂ is the unsubstituted alkyl, then R₅ represents carboxy(C₁-C₁₂)alkyl.

In another embodiment alkyl is unsubstituted or substituted by one or more of the following groups, CN, (C₁-C₁₂)alkoxyC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine, with the proviso that when R₂ is the unsubstituted alkyl, then R₅ represents carboxy(C₁-C₁₂)alkyl.

In a further embodiment alkyl is unsubstituted or substituted by one or more of the following groups, CN, (C₁-C₁₂)alkoxyC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine, or any one of the groups

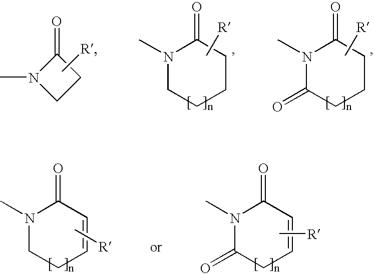

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms, with the proviso that when R₂ is the unsubstituted alkyl, then R₅ represents carboxy(C₁-C₁₂)alkyl.

The term “alkyl” includes both linear or branched chain groups.

In R^{d} any “alkyl” generally is optionally substituted with one or more halogens (F, Cl, Br or I). E.g. (C₁-C₁₂)alkylthio may be embodified by —SCF₃.

The term “cycloalkyl” generally denotes a substituted or unsubstituted (C₃-C₆), unless other chain length specified, cyclic hydrocarbon.

In one embodiment cycloalkyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “alkoxy” includes both linear or branched chain groups.

The term aryl denotes a substituted or unsubstituted (C₆-C₁₄) aromatic hydrocarbon and includes, but is not limited to, phenyl, naphthyl, tetrahydronaphtyl, indenyl, indanyl, antracenyl, fenantrenyl, and fluorenyl.

In one embodiment aryl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic hetorocyclic groups, and includes, but is not limited to azetidine, oxetan, furan, thiophene, pyrrole, pyrroline, pyrrolidine, 2-oxopyrrolidine, 2,5-dioxopyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, pyridine as well as pyridine-N-oxide, piperidine, 2-oxopiperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzothiadiazole, imidazothiazole, 2,3-dihydrobenzofuran, isoxazole, 3-benzisoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, eg. R₄ when selected as heterocyclyl may be a furan, when R^{d} (also when selected as heterocyclyl) may be a pyrrole.

In one embodiment of the invention the term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic hetorocyclic groups, and includes, but is not limited to azetidine, furan, thiophene, pyrrole, pyrroline, pyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, pyridine as well as pyridine-N-oxide, piperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzothiadiazole, imidazothiazole, 2,3-dihydrobenzofuran, isoxazole, 3-benzisoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, eg. R₄ when selected as heterocyclyl may be a furan, when R^{d} (also when selected as heterocyclyl) may be a pyrrole.

In one embodiment heterocyclyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

In another embodiment of the invention the heterocyclyl group comprises an aromatic 5-membered or 6-membered heterocyclic ring containing one, two or three heteroatoms selected from nitrogen, oxygen and sulphur, and an aromatic 5-membered or 6-membered heterocyclic ring containing one, two or three heteroatoms selected from nitrogen, oxygen and sulphur which is fused to a benzene ring;

In an alternative embodiment of the invention the heterocyclyl group is a non-aromatic 5-membered or 6-membered heterocyclic ring containing one, two or three heteroatoms selected from nitrogen, oxygen and sulphur, fused to a benzene ring.

In a further embodiment of the invention the heterocyclyl group is a group chosen among furyl, pyrrolyl, thienyl, pyridyl, N-oxido-pyridyl, pyrazinyl, pyrimidinyl, pyridazinyl, imidazolyl, oxazolyl, isooxazolyl, thiazolyl, isothiazolyl, oxadiazolyl, 1,2,3-triazolyl, 1,2,4-triazolyl, benzfuranyl, quinolyl, isoquinolyl, benzimidazolyl, indolyl, benzdihydrofuranyl, benzodioxolyl (such as 1,3-benzodioxolyl), benzoxadiazole, dihydrobenzodioxin, benzothiophene, benzothiadiazole, imidazothiazole, 2,3-dihydrobenzofuran, isoxazole, dihydropyrazole and benzdioxanyl (such as 1,4-benzdioxanyl). More particular values include, for example, furyl, pyrrolyl, thienyl, pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole, dihydropyrazole and benzdioxanyl (such as 1,4-benzdioxanyl).

In an even further embodiment of the invention the heterocyclyl group is a group chosen among furyl, pyrrolyl, thienyl, pyridyl, N-oxido-pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole or dihydropyrazole.

In one embodiment of the invention R₁ represents R₇C(O).

In another embodiment of the invention R₁ represents R₁₇S.

In a further embodiment of the invention R₁ represents R₁₈C(S).

In yet another embodiment R₁ represents a group (gII),

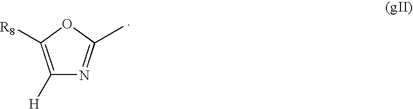

R₁ may also be embodified by the group gII,

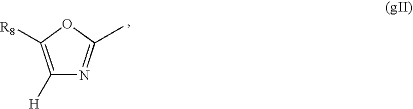

in which R₈ is selected from H, (C₁-C₆)alkyl, such as methyl or ethyl.

In another embodiment for the group R₈ this group can be chosen among hydrogen, methyl, ethyl, n-propyl and n-butyl.

In one embodiment R₂ represents substituted (C₁-C₁₂)alkyl optionally interrupted by sulphur, substituted (C₁-C₁₂)alkoxy or substituted (C₁-C₁₂)alkylthio, wherein any one of these groups is substituted by one or more of azido, carboxy, cyano, (C₁-C₁₂)alkylcarbonyloxy, hydroxy(C₁-C₁₂)alkylcarbonyloxy, arylcarbonyloxy, heterocyclylcarbonyloxy, (C₁-C₁₂)alkyloxycarbonyl, (C₁-C₁₂)alkyl(C(S)), (C₁-C₁₂)alkyl(S(CO)), (C₁-C₁₂)alkylthio, hydroxy(C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl, heterocyclyl(C₁-C₁₂)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₁₂)alkyl, (C₁-C₁₂)alkylcarbonyl or R^{a(2)} and R^{b(2)} together with the nitrogen atom represent piperidine, pyrrolidine, azetidine or aziridine or any one of the groups

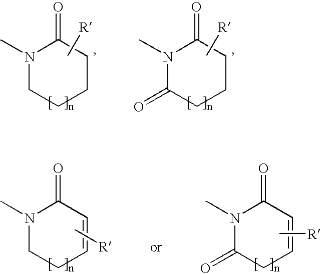

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;

Further R₂ represents substituted (C₁-C₁₂)alkoxy or substituted (C₁-C₁₂)alkylthio, wherein any one of these groups is substituted by one or more of any one of OH, aryl, (C₃-C₆)cycloalkyl or heterocyclyl; Further R₂ represents (C₁-C₁₂)alkylthio, substituted by one or more halogen (F, Cl, I, Br) atom(s); Further R₂ represents (C₁-C₁₂)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₁₂)alkoxy, (C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl and heterocyclyl(C₁-C₁₂)alkylcarbonyl; Further R₂ represents unsubstituted (C₁-C₁₂)alkyl with the proviso that at the same time R₅ represents carboxy(C₁-C₁₂)alkyl; Further R₂ represents a group of formula ((R^{a(2)})N(R^{b(2)}))(CO)—, in which R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine.

In another embodiment of the invention R₂ represents substituted (C₁-C₁₂)alkyl optionally interrupted by sulphur, substituted (C₁-C₁₂)alkoxy or substituted (C₁-C₁₂)alkylthio, wherein any one of these groups is substituted by one or more of azido, carboxy, cyano, (C₁-C₁₂)alkylcarbonyloxy, hydroxy(C₁-C₁₂)alkylcarbonyloxy, arylcarbonyloxy, heterocyclylcarbonyloxy, (C₁-C₁₂)alkyloxycarbonyl, (C₁-C₁₂)alkyl(C(S)), (C₁-C₁₂)alkyl(S(CO)), (C₁-C₁₂)alkylthio, hydroxy(C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl, heterocyclyl(C₁-C₁₂)alkylcarbonyl or of a group of formula NR^{a(2)}R^{b(2)} or —(CO)NR^{a(2)}R^{b(2)} in which R^{a(2)} and R^{b(2)} each and independently represent H, (C₁-C₁₂)alkyl, (C₁-C₁₂)alkylcarbonyl or R^{a(2)} and R^{b(2)} together with the nitrogen atom represent piperidine, pyrrolidine, azetidine or aziridine or any one of the groups

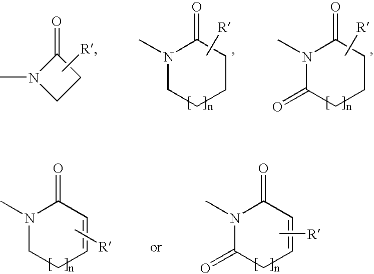

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;

Further R₂ represents substituted (C₁-C₁₂)alkoxy or substituted (C₁-C₁₂)alkylthio, wherein any one of these groups is substituted by one or more of any one of OH, aryl, (C₃-C₆)cycloalkyl or heterocyclyl; Further R₂ represents (C₁-C₁₂)alkylthio, substituted by one or more halogen (F, Cl, I, Br) atom(s); Further R₂ represents (C₁-C₁₂)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₁₂)alkoxy, (C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl and heterocyclyl(C₁-C₁₂)alkylcarbonyl; Further R₂ represents a group of formula ((R^{a(2)})N(R^{b(2)}))(CO)—, in which R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;

In a further embodiment R₂ represents unsubstituted (C₁-C₁₂)alkyl with the proviso that at the same time R₅ represents carboxy(C₁-C₁₂)alkyl;

In an even further embodiment, R₂ represents methyl substituted by any one of the groups

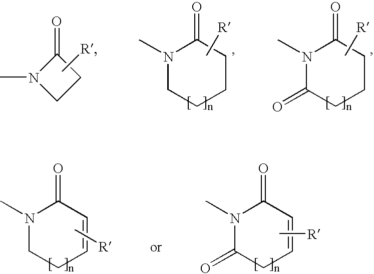

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms; or methyl substituted by (C₁-C₆)alkylcarbonyloxy, a group NR^{a(2)}R^{b(2)} wherein R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine; (C₁-C₆)alkoxycarbonyl(C₁-C₆)alkyl, (C₁-C₆)alkylcarbonyl(C₁-C₆)alkyl, azido, (C₁-C₆)alkylsulfonyl, heterocyclylthio, heterocyclyl(C₁-C₆)alkylthio, (C₁-C₆)alkoxycarbonyl(C₁-C₆)alkylthio, a group NR^{a(2)}R^{b(2)}carbonyl(C₁-C₆)alkylthio wherein R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine; hydroxy(C₁-C₆)alkylcarbonyloxy, (C₁-C₁₂)alkylthio, hydroxy(C₁-C₁₂)alkylthio or (C₁-C₆)alkylcarbonylamino(C₁-C₆)alkylthio.

In an alternative further embodiment, R₂ represents methyl substituted by any one of the groups

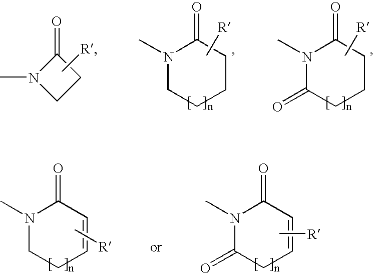

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;

In another alternative further embodiment, R₂ represents methyl substituted by any one of the groups

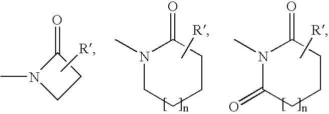

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;

In another special alternative embodiment, R₂ represents any one of the groups 2-oxo-piperidin-1-yl-methyl and 2-oxo-pyrrolidin-1-yl-methyl.

In a further special alternative embodiment, R₂ represents 2-oxo-piperidin-1-yl-methyl.

In an even further special alternative embodiment, R₂ represents 2-oxo-pyrrolidin-1-yl-methyl.

In an even further embodiment, R₂ represents —S—R″ wherein R″ represents hydroxy(C₁-C₁₂)alkyl, (C₁-C₆)alkylcarbonylamino(C₁-C₆)alkyl, carboxy(C₁-C₆)alkyl, (C₁-C₆)alkyloxycarbonyl(C₁-C₆)alkyl, a group NR^{a(2)}R^{b(2)}carbonyl(C₁-C₆)alkyl wherein R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine.

In another further embodiment, R₂ represents —O—R′″, wherein R′″ represents (C₁-C₆)alkylcarbonyl, (C₁-C₆)alkyloxycarbonyl(C₁-C₆)alkyl, cyano(C₁-C₆)alkyl, hydroxy(C₁-C₁₂)alkyl, (C₁-C₆)alkylcarbonylamino(C₁-C₆)alkyl, heterocyclyl(C₁-C₆)alkyl, carboxy(C₁-C₆)alkyl, (C₁-C₆)alkylaminocarbonyl(C₁-C₆)alkyl, (C₁-C₆)alkylcarbonyl(C₁-C₆)alkyl, a group NR^{a(2)}R^{b(2)}carbonyl(C₁-C₆)alkyl wherein R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine.

Embodiments for R₃ include, for example, H, methyl, methylsulfinyl, hydroxymethyl, methoxy or amino unsubstituted or optionally substituted with one or two methyl groups.

Other embodiments for R₃ include H or amino unsubstituted or optionally substituted with one or two methyl groups.

In a further embodiment of the invention R₃ is H.

Embodiments for R₄ include H, halogen such as chloro, methyl, cyano, nitro, amino unsubstituted or optionally substituted with one or two methyl groups and further includes 4-methoxy-4-oxobutoxy, 3-carboxy-propoxy and methylcarbonyl.

In a specific embodiment of the invention R₄ is selected from the group consisting of hydrogen, cyano, fluoro, chloro, bromo and iodo.

In a further specific embodiment of the invention R₄ is selected from the group consisting of cyano, fluoro, chloro, bromo and iodo.

In an even further specific embodiment of the invention R₄ is selected from the group consisting of cyano and chloro.

In another even further specific embodiment of the invention R₄ is selected from the group consisting of fluoro, cyano and chloro.

In one embodiment R₅ represents hydrogen or methyl, with the proviso that when R₂ is unsubstituted alkyl, then R₅ represents carboxy(C₁-C₁₂)alkyl.

In an alternative embodiment R₅ is hydrogen, with the proviso that when R₂ is unsubstituted alkyl, then R₅ represents carboxy(C₁-C₁₂)alkyl.

In another embodiment R₅ represents carboxy(C₁-C₁₂)alkyl. In a further embodiment R₅ represents carboxy(C₁-C₆)alkyl. In an even further embodiment R₅ represents carboxymethyl.

In an alternative further embodiment R₅ represents hydrogen or carboxy(C₁-C₆)alkyl, with the proviso that when R₂ is unsubstituted alkyl, then R₅ represents carboxy(C₁-C₆)alkyl.

In an even further alternative embodiment R₅ represents hydrogen.

In one embodiment of the invention R₇ represents (C₁-C₁₂)alkyl optionally interrupted by oxygen, and/or optionally substituted by OH, aryl, cycloalkyl, heterocyclyl or one or more halogen (F, Cl, Br, I) atoms; further R₇ represents (C₂-C₁₂)alkenyl optionally interrupted by oxygen, and/or optionally substituted by OH, aryl, cycloalkyl, heterocyclyl or one or more halogen (F, Cl, Br, I) atoms; further R₇ represents (C₂-C₁₂)alkynyl optionally interrupted by oxygen, and/or optionally substituted by OH, aryl, cycloalkyl, heterocyclyl or one or more halogen (F, Cl, Br, I) atoms;

In another embodiment of the invention R₇ represents (C₁-C₆)alkyl optionally interrupted by oxygen, and/or optionally substituted by OH, aryl, cycloalkyl, heterocyclyl or one or more halogen (F, Cl, Br, I) atoms; further R₇ represents (C₂-C₆)alkenyl optionally interrupted by oxygen, and/or optionally substituted by OH, aryl, cycloalkyl, heterocyclyl or one or more halogen (F, Cl, Br, I) atoms; further R₇ represents (C₂-C₆)alkynyl optionally interrupted by oxygen, and/or optionally substituted by OH, aryl, cycloalkyl, heterocyclyl or one or more halogen (F, Cl, Br, I) atoms;

In an alternative embodiment of the invention R₇ represents (C₁-C₁₂)alkyl optionally substituted by OH, aryl, cycloalkyl, heterocyclyl or one or more halogen (F, Cl, Br, I) atoms;

In an even further alternative embodiment of the invention R₇ represents (C₁-C₁₂)alkyl optionally substituted by OH or one or more halogen (F, Cl, Br, I) atoms;

In another embodiment of the invention R₇ represents (C₃-C₆)cycloalkyl, hydroxy(C₁-C₁₂)alkyl, aryl or heterocyclyl;

In one embodiment of the invention R₈ represents (C₁-C₁₂)alkyl optionally interrupted by oxygen, and/or optionally substituted by OH, aryl, cycloalkyl, heterocyclyl or one or more halogen (F, Cl, Br, I) atoms;

In another embodiment of the invention R₈ represents (C₁-C₁₂)alkyl optionally substituted by OH or one or more halogen (F, Cl, Br, I) atoms;

Embodiments for R₁₄ include, for example, hydrogen, methyl, amino, tert-butyloxycarbonyl, tert-butyloxycarbonyl-imino, 2-carboxyethyl and 3-tert-butoxy-3-oxo-propyl.

Other further embodiments for R₁₄ include, for example, hydrogen, methyl, tert-butyloxycarbonyl-imino, and amino.

In one embodiment of the invention R₁₅ represents H.

In a further embodiment of the invention both R₁₄ and R₁₅ represents H.

In one embodiment of the invention R₁₇ represents (C₁-C₁₂)alkyl, optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₁₂)alkyl, aryl or heterocyclyl.

In another embodiment of the invention R₁₇ represents (C₁-C₆)alkyl, optionally substituted by OH, aryl, cycloalkyl, heterocyclyl or one or more halogen (F, Cl, Br, I) atoms.

In a further embodiment of the invention R₁₇ represents (C₁-C₆)alkyl.

In one embodiment of the invention R₁₈ represents (C₁-C₁₂)alkyl, optionally interrupted by oxygen and/or optionally substituted by OH, aryl, cycloalkyl, heterocyclyl or one or more halogen (F, Cl, Br, I) atoms; further R₁₈ represents (C₃-C₆)cycloalkyl, hydroxy(C₁-C₁₂)alkyl, aryl or heterocyclyl.

In another embodiment of the invention R₁₈ represents (C₁-C₆)alkyl, optionally substituted by OH, aryl, cycloalkyl, heterocyclyl or one or more halogen (F, Cl, Br, I) atoms.

In a further embodiment of the invention R₁₈ represents (C₁-C₆)alkyl.

In one embodiment R^{d} represents (C₁-C₁₂)alkyl, (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

In one embodiment R^{d} represents (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, halogen substituted (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

Further embodiments for R^{d} includes aryl or heterocyclyl, more particularly, aryl or aromatic heterocyclyl.

Another embodiment for R^{d} include, aryl such as phenyl and aromatic heterocyclyl such as thienyl.

Other embodiments of R^{d} include phenyl which optionally may be substituted.

In a special embodiment R^{d} represents aryl, heterocyclyl or (C₃-C₆)cycloalkyl, and anyone of these groups are optionally substituted with one or more halogen (F, Cl, Br, I) atoms or mixed halogen atoms, and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine.

In a further special embodiment R^{d} represents aryl or (C₃-C₆)cycloalkyl, and anyone of these groups are optionally substituted with one or more halogen (F, Cl, Br, I) atoms or mixed halogen atoms, and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, halogen substituted (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;

Even further embodiments for R^{d} include phenyl optionally substituted at the 2,3,4,5 or 6-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2,3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl, 2,3-dihydro-1,4-benzodioxin-6-yl, 5-chloro-3-methyl-1-benzothien-2-yl, 2,1,3-benzothiadiazol-4-yl, 2,5-dimethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, 5-bromo-2-thienyl, 5-pyridin-2-yl-2-thienyl, 2,5-dichloro-3-thienyl, 4,5-dichloro-2-thienyl, benzothien-3-yl, 2,5-dimethyl-3-thienyl, 3-thienyl, 2-thienyl, 5-methylisoxazol-4-yl, pyridin-3-yl, [1-methyl-5-(trifluoromethyl)-1H-pyrazol-3-yl]-2-thienyl, 5-chloro-1,3-dimethyl-1H-pyrazol-4-yl, 4-[(4-chlorophenyl)sulfonyl]-3-methyl-2-thienyl, 5-(methoxycarbonyl)-2-furyl and 4-(methoxycarbonyl)-5-methyl-2-furyl.

Even other further embodiments for R^{d} include phenyl optionally substituted at the 2,3,4 or 5-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2,3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl, 2,3-dihydro-1,4-benzodioxin-6-yl, 5-chloro-3-methyl-1-benzothien-2-yl, 2,1,3-benzothiadiazol-4-yl, 2,5-dimethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, 5-bromo-2-thienyl, 5-pyridin-2-yl-2-thienyl, 2,5-dichloro-3-thienyl, 4,5-dichloro-2-thienyl, benzothien-3-yl, 2,5-dimethyl-3-thienyl, 3-thienyl, 2-thienyl, 5-methylisoxazol-4-yl, pyridin-3-yl, [1-methyl-5-(trifluoromethyl)-1H-pyrazol-3-yl]-2-thienyl, 5-chloro-1,3-dimethyl-1H-pyrazol-4-yl, 4-[(4-chlorophenyl)sulfonyl]-3-methyl-2-thienyl, 5-(methoxycarbonyl)-2-furyl and 4-(methoxycarbonyl)-5-methyl-2-furyl.

In one embodiment of the invention R^{c} represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, C1, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-(C₁-C₄)alkylene group with any substituents according to above.

In another embodiment of the invention R^{c} represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

In an alternative embodiment of the invention R^{c} is a single bond.

In a further alternative embodiment of the invention R^{c} represents imino (—NH—) or substituted imino (—NR₁₉—), wherein R₁₉ represents H or (C₁-C₄)alkyl;

In a preferred embodiment of the invention R^{c} represents an unsubstituted or monosubstituted or disubstituted (C₁-C₃)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-(C₁-C₃)alkylene group with any substituents according to above.

In a further embodiment of the invention R^{c} represents an unsubstituted or monosubstituted or disubstituted (C₁-C₄)alkylene group wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl, i.e. R^{c}R^{d} represents a heterocyclyl-(C₁-C₄)alkylene group with any substituents according to above.

In a further preferred embodiment of the invention R^{c} represents an unsubstituted or monosubstituted or disubstituted (C₁-C₃)alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents heterocyclyl, i.e. R^{c}R^{d} represents a heterocyclyl-(C₁-C₃)alkylene group with any substituents according to above.

In a particular embodiment of the invention R^{c} represents a C₁-alkylene group wherein any substituents each individually and independently are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine, and R^{d} represents aryl, i.e R^{c}R^{d} represents an aryl-C₁-alkylene group with any substituents according to above.

In a further particular embodiment of the invention R^{c} represents an unsubstituted or monosubstituted or disubstituted methylene group, imino (—NH—) or methylimino (—N(CH₃)—), wherein any substituents each and individually are selected from (C₁-C₄)alkyl, (C₁-C₄)alkoxy, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine.

In another further particular embodiment of the invention R^{c} represents an unsubstituted or monosubstituted or disubstituted methylene group, imino (—NH—) or methylimino (—N(CH₃)—), wherein any substituents each and individually are selected from (C₁-C₄)alkyl.

In one embodiment of the invention R₁₉ represents hydrogen.

In another embodiment of the invention R₁₉ represents methyl.

In a most particular embodiment of the invention R^{c}R^{d} represents a benzyl group, or a benzyl group which is substituted according to what is described in connection to substitution of the aryl group.

In one embodiment of the invention X represents a single bond.

In another embodiment of the invention X represents imino (—NH—) or methylene (—CH₂—).

In yet another embodiment X represents imino (—NH—).

In a further embodiment X represents methylene (—CH₂—).

Suitable values for the B ring/ring system include, for example, diazepanylene, piperazinylene, piperidinylene, pyrrolidinylene and azetidinylene, wherein anyone of them may be presents in any of their isomeric forms (e.g. piperazin-tetrahydropyridazin-tetrahydropyrimidin).

Embodiments for the B ring/ring system include, for example, diazepanylene, piperazinylene, piperidinylene, pyrrolidinylene and azetidinylene.

Further embodiments include these groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₁₂)alkyl optionally substituted by one or more of halogen (F, Cl, Br, I) or mixed halogen atoms, OH, aryl, cycloalkyl and heterocyclyl.

In an alternative to the embodiment for the B ring/ring system above, the embodiment include, for example, diazepanylene, piperazinylene, piperidinylene, pyrrolidinylene or azetidinylene groups which are substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₆)alkyl optionally substituted by one or more of halogen (F, Cl, Br, I) or mixed halogen atoms, OH, aryl, cycloalkyl and heterocyclyl.

In a further alternative to the embodiment for the B ring/ring system above, B is chosen from an azetidinylene group or a piperidinylene group, any of which optionally is substituted with R₁₄ having a (C₁-C₆)alkyl group, wherein the (C₁-C₆)alkyl group optionally is substituted with OH, COOH or COOR^{e} group(s), e.g. a 2-carboxyethyl group, and wherein R^{e} represents H, aryl, cycloalkyl, heterocyclyl or (C₁-C₆)alkyl optionally substituted by one or more of halogen (F, Cl, Br, I) or mixed halogen atoms, OH, aryl, cycloalkyl and heterocyclyl.

In an even further alternative to the embodiment for the B ring/ring system above, B is chosen from an unsubstituted azetidinylene group or an unsubstituted piperidinylene group.

In a special alternative to the embodiment for the B ring/ring system above, B is an unsubstituted piperidinylene group.

In a further special alternative to the embodiment for the B ring/ring system above, B is an unsubstituted azetidinylene group.

A 2nd embodiment of formula I is defined by;

R₁ represents R₇C(O), R₁₇S, R₁₈C(S) or a group gII

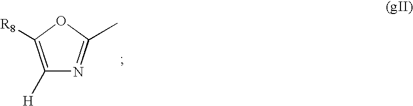

preferably R₁ represents R₇C(O) or the group (gII)

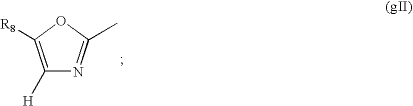

R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups

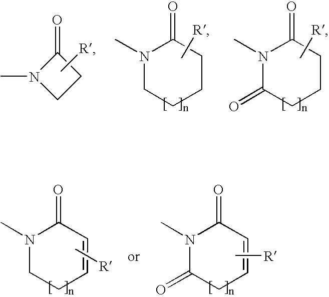

wherein n is an integer chosen from 0, 1 and 2, and R′ is H, CN, OH, a halogen (F, Cl, Br, I) atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;

Further R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; Further R₂ represents (C₁-C₆)alkylthio, substituted by one or more halogen (F, Cl, I, Br) atom(s); Further R₂ represents (C₁-C₆)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; Further R₂ represents unsubstituted (C₁-C₆)alkyl with the proviso that at the same time R₅ represents carboxy(C₁-C₆)alkyl; Further R₂ represents a group of formula ((R^{a(2)})N(R^{b(2)}))(CO)—, in which R^{a(2)} and R^{b(2)} each and independently represent H, (C₁-C₆)alkyl, aryl, aryl(C₁-C₆)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen (F, Cl, Br, I) atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, (C₁-C₆)alkoxycarbonyl, aryl, cycloalkyl, heterocyclyl or one or more halogen (F, Cl, Br, I) atoms; further R₄ represents (C₃-C₆)cycloalkyl, hydroxy(C₁-C₆)alkyl, (C₁-C₆)alkylC(O), (C₁-C₆)alkylcycloalkyl, (C₁-C₆)alkoxy wherein the alkoxygroup may optionally be substituted by one or more halogen (F, Cl, Br, I) atoms, OH and/or COOH and/or (C₁-C₆)alkoxycarbonyl; further R₄ represents (C₁-C₆)alkylthioC(O), (C₁-C₆)alkylC(S), (C₁-C₆)alkoxyC(O), (C₃-C₆)cycloalkoxy, aryl, aryl(C₁-C₆)alkoxy, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl, (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₅ represents H or (C₁-C₆)alkyl or carboxy(C₁-C₆)alkyl; with the proviso that when R₂ is unsubstituted (C₁-C₆)alkyl, R₅ represents carboxy(C₁-C₆)alkyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms; further R₇ represents (C₂-C₆)alkenyl optionally interrupted by oxygen, and/or optionally substituted by OH, aryl, cycloalkyl, heterocyclyl or one or more halogen (F, Cl, Br, I) atoms; further R₇ represents (C₂-C₆)alkynyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, aryl(C₁-C₆)alkoxy, aryl(C₁-C₆)alkyl, (C₃-C₆)cycloalkyl(C₁-C₆)alkoxy, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl or (C₃-C₆)cycloalkyl(C₁-C₆)alkylsulfonyl, a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, aryl(C₁-C₆)alkoxy, aryl(C₁-C₆)alkyl, (C₃-C₆)cycloalkyl(C₁-C₆)alkoxy, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O)), (C₁-C₆)alkoxyC(O) or R^{a(}15) and R^{b(15)} together with the nitrogen atom represent piperidine, pyrrolidine, azetidine or aziridine;

R₁₇ represents (C₁-C₆)alkyl optionally interrupted by oxygen and/or optionally substituted by OH, aryl, cycloalkyl, heterocyclyl or one or more halogen (F, Cl, Br, I) atoms; further R₁₇ represents (C₃-C₆)cycloalkyl, hydroxy(C₁-C₆)alkyl, (C₁-C₆)alkoxy, (C₃-C₆)cycloalkoxy, aryl or heterocyclyl;

R₁₈ represents (C₁-C₆)alkyl optionally interrupted by oxygen and/or optionally substituted by OH, aryl, cycloalkyl, heterocyclyl or one or more halogen (F, Cl, Br, I) atoms; further R₁₈ represents (C₃-C₆)cycloalkyl, hydroxy(C₁-C₆)alkyl, (C₁-C₆)alkoxy, (C₃-C₆)cycloalkoxy, aryl or heterocyclyl;

R^{c} is a single bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₆)alkyl, (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, halogen substituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 3rd embodiment of formula I is defined by;

R₁ represents R₇C(O), or a group gII

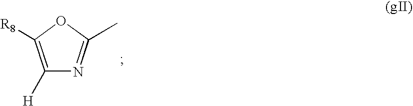

R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups

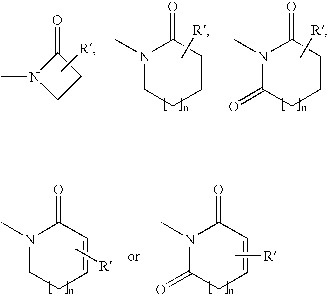

wherein n is an integer chosen from 0, 1 and 2, and R′ is H, CN, OH, a halogen (F, Cl, Br, I) atom, or one of the groups (C₁-C₆)alkyl, aryl, (C₁-C₆)alkoxy, (C₁-C₆)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;

Further R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; Further R₂ represents (C₁-C₆)alkylthio, substituted by one or more halogen (F, Cl, I, Br) atom(s); Further R₂ represents (C₁-C₆)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; Further R₂ represents unsubstituted (C₁-C₆)alkyl with the proviso that at the same time R₅ represents carboxy(C₁-C₆)alkyl; Further R₂ represents a group of formula ((R^{a(2)})N(R^{b(2)}))(CO)—, in which R^{a(2)} and R^{b(2)} each and independently represent H, (C₁-C₆)alkyl, aryl, aryl(C₁-C₆)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₁-C₆)alkoxy optionally substituted by one or more halogen (F, Cl, Br, I) atoms; further R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxygroup may optionally be substituted by one or more halogen (F, Cl, Br, I) atoms, OH and/or COOH and/or methoxycarbonyl; further R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₅ represents H or (C₁-C₆)alkyl or carboxy(C₁-C₆)alkyl; with the proviso that when R₂ is unsubstituted (C₁-C₆)alkyl, R₅ represents carboxy(C₁-C₆)alkyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms; further R₇ represents (C₂-C₆)alkenyl optionally interrupted by oxygen, and/or optionally substituted by OH, aryl, cycloalkyl, heterocyclyl or one or more halogen (F, Cl, Br, I) atoms; further R₇ represents (C₂-C₆)alkynyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl, hydroxy(C₁-C₆)alkyl, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl or heterocyclyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, aryl(C₁-C₆)alkoxy, aryl(C₁-C₆)alkyl, (C₃-C₆)cycloalkyl(C₁-C₆)alkoxy, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, aryl(C₁-C₆)alkoxy, aryl(C₁-C₆)alkyl, (C₃-C₆)cycloalkyl(C₁-C₆)alkoxy, heterocyclyl, a halogen (F, Cl, Br, I) atom, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a}(15) and R^{b(15)} independently represent H, (C₁-C₆)alkyl, (C₁-C₆)alkylC(O), (C₁-C₆)alkoxyC(O) or R^{a}(15) and R^{b(15)} together with the nitrogen atom represent piperidine, pyrrolidine, azetidine or aziridine;

R^{c} is a single bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₆)alkyl, (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, halogen substituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 4rth embodiment of formula I is defined by;

R₁ represents R₇C(O) or a group gII

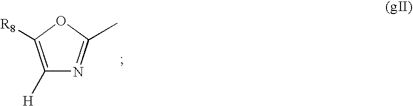

R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups

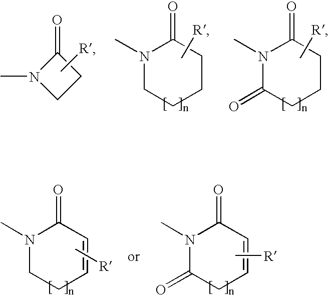

wherein n is an integer chosen from 0, 1 and 2, and R′ is H, CN, OH, a halogen (F, Cl, Br, I) atom, or one of the groups (C₁-C₄)alkyl, aryl, (C₁-C₄)alkoxy, (C₁-C₄)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen (F, Cl, Br, I) atoms;

Further R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; Further R₂ represents (C₁-C₆)alkylthio, substituted by one or more halogen (F, Cl, I, Br) atom(s); Further R₂ represents (C₁-C₆)alkylcarbonyloxy, aryl carbonyloxy, heterocyclylcarbonyloxy of which any one optionally is substituted by one or more of any one of the following groups or atoms; azido, cyano, halogen (F, Cl, Br, I) atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; Further R₂ represents unsubstituted (C₁-C₆)alkyl with the proviso that at the same time R₅ represents carboxy(C₁-C₆)alkyl;

R₃ represents H or a group of formula NR^{a (3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents CN, halogen (F, Cl, Br, I), further R₄ represents (C₁-C₆)alkylC(O), (C₁-C₆)alkoxy wherein the alkoxygroup may optionally be substituted by one or more halogen (F, Cl, Br, I) atoms, OH and/or COOH and/or methoxycarbonyl;

R₅ represents H or carboxy(C₁-C₆)alkyl; with the proviso that when R₂ is unsubstituted (C₁-C₆)alkyl, R₅ represents carboxy(C₁-C₆)alkyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms; further R₇ represents(C₂-C₆)alkenyl optionally interrupted by oxygen, and/or optionally substituted by OH, aryl, cycloalkyl, heterocyclyl or one or more halogen (F, Cl, Br, I) atoms; further R₇ represents (C₂-C₆)alkynyl optionally interrupted by oxygen, and/or optionally substituted by OH, aryl, cycloalkyl, heterocyclyl or one or more halogen (F, Cl, Br, I) atoms; further R₇ represents (C₃-C₆)cycloalkyl or hydroxy(C₁-C₆)alkyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;

R₁₅ represents H;

R^{c} represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno (F, Cl, Br, I), hydroxyl, NR^{a}(R^{c})R^{b}(R^{c}) in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; Further R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above; preferably R^{c} represents imino or (C₁-C₄)alkyleneimino or an unsubstituted or monosubstituted or polysubstituted (C₁-C₄)alkylene group or (C₁-C₄)oxoalkylene group with any substituents according to above;

R₁₉ represents H or (C₁-C₄)alkyl;

R^{d} represents (C₁-C₆)alkyl, (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, halogen substituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; further X may represent a group (—CH₂-)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 5th embodiment of formula I is defined by that;

R₁ is chosen from a group consisting of methylcarbonyl, ethylcarbonyl, n-propylcarbonyl, isopropylcarbonyl, cyclopropylcarbonyl, n-butylcarbonyl, 4-buten-1-ylcarbonyl, 3,3,3-trifluoropropylcarbonyl and 5-ethyl-1,3-oxazol-2-yl;

R₂ is chosen from a group consisting of (2-oxopyrrolidin-1-yl)methyl and (2-oxopiperidin-1-yl)methyl,

R₃ is H;

R₄ is chosen from a group consisting of fluoro, chloro and cyano;

R₅ is H or methyl;

R₇ is chosen from a group consisting of methyl, ethyl, n-propyl, isopropyl, cyclopropyl, n-butyl, 4-buten-1-yl and 3,3,3-trifluoropropyl;

R₈ is ethyl;

R₁₄ is H;

R₁₅ is H;

R^{c} is chosen from a group consisting of methylene (—CH₂—), methylmethine (—CH(CH₃)—), imino (—NH—) and methylimino (—N(CH₃)—);

R₁₉ is chosen from H or methyl;

R^{d} is chosen from a group consisting of cyclobutyl, cyclopentyl, cyclohexyl, phenyl, 4-methylphenyl, 4-isopropylphenyl, 4-methoxyphenyl, 4-fluorophenyl, 4-chlorophenyl, 2,4-difluorophenyl, and 4-cyanophenyl;

X represents a single bond;

B is 4-piperidin-1-ylene and the substituents R₁₄ and R₁₅ are connected to the B ring/ring system, in such a way that no quaternary ammonium compounds are formed (by these connections).

In a 6th embodiment of formula (I), formula (I) is defined as being any compound(s) of formula (Ia)-(Ii):

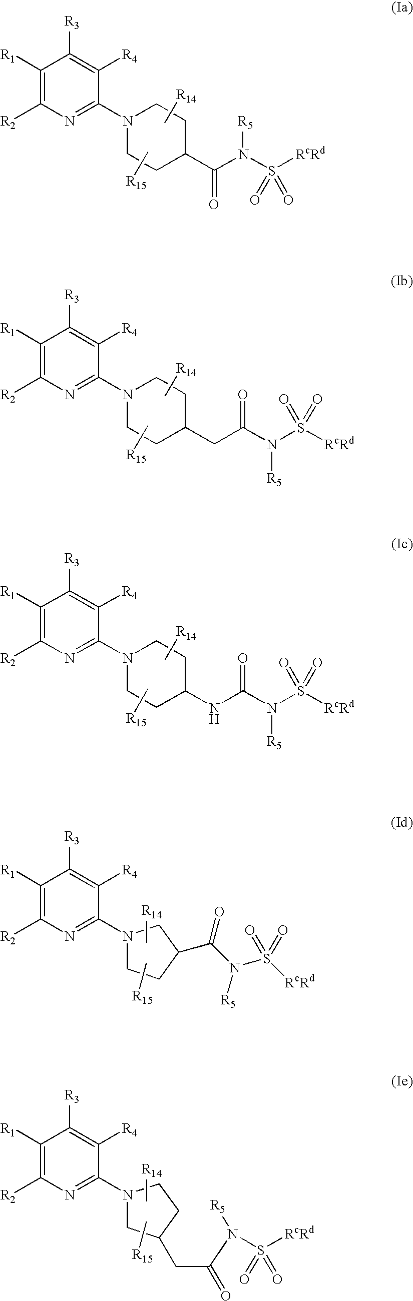

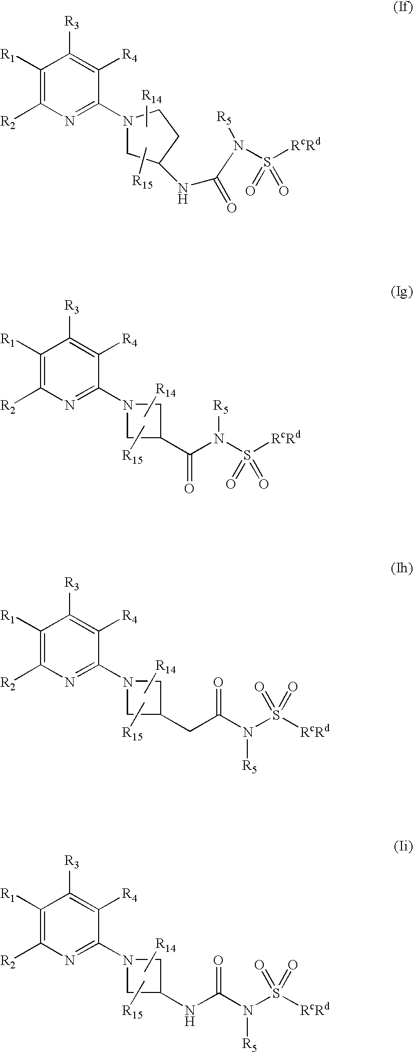

In the above Ia to Ii the various values of R are as defined above and include any of the previously mentioned embodiments.

In a 7^{th} embodiment formula (I) is defined as being any compound(s) of formula (Iaa)-(Igg);

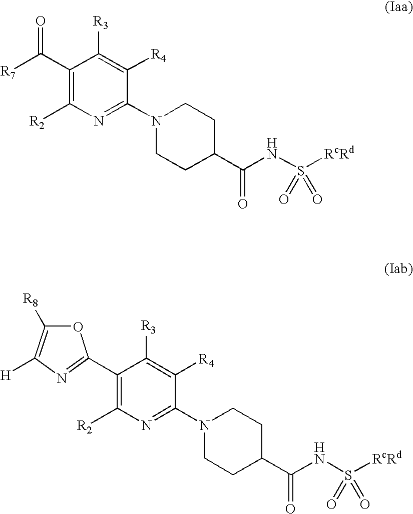

In the above Iaa to Iab the various values of R (except R₁₄ and R₁₅ being H) are as defined above and include any of the previously mentioned embodiments.

Examples of specific compounds according to the invention can be selected from;
1-{5-acetyl-3-cyano-6-[(2-oxopyrrolidin-1-yl)methyl]pyridin-2-yl}-N-(benzylsulfonyl)piperidine-4-carboxamide
N-(benzylsulfonyl)-1-{5-butyryl-3-cyano-6-[(2-oxopyrrolidin-1-yl)methyl]pyridin-2-yl}piperidine-4-carboxamide
1-{5-acetyl-3-cyano-6-[(2-oxopiperidin-1-yl)methyl]pyridin-2-yl}-N-(benzylsulfonyl)piperidine-4-carboxamide
1-{5-butyryl-3-cyano-6-[(2-oxopiperidin-1-yl)methyl]pyridin-2-yl}-N-[(4-methylbenzyl)sulfonyl]piperidine-4-carboxamide
1-{5-butyryl-3-cyano-6-[(2-oxopyrrolidin-1-yl)methyl]pyridin-2-yl}-N-[(4-methylbenzyl)sulfonyl]piperidine-4-carboxamide
1-{5-butyryl-3-cyano-6-[(2-oxopyrrolidin-1-yl)methyl]pyridin-2-yl}-N-[(2,4-difluorobenzyl)sulfonyl]piperidine-4-carboxamide
1-{5-butyryl-3-cyano-6-[(2-oxopyrrolidin-1-yl)methyl]pyridin-2-yl}-N-[(cyclopentylmethyl)sulfonyl]piperidine-4-carboxamide
N-(benzylsulfonyl)-1-{3-cyano-5-(cyclopropylcarbonyl)-6-[(2-oxopyrrolidin-1-yl)methyl]pyridin-2-yl}piperidine-4-carboxamide
1-{3-cyano-5-(cyclopropylcarbonyl)-6-[(2-oxopyrrolidin-1-yl)methyl]pyridin-2-yl}-N-[(cyclopentylmethyl)sulfonyl]piperidine-4-carboxamide
N-(benzylsulfonyl)-1-{3-cyano-6-[(2-oxopyrrolidin-1-yl)methyl]-5-propionylpyridin-2-yl}piperidine-4-carboxamide
1-{3-cyano-6-[(2-oxopyrrolidin-1-yl)methyl]-5-propionylpyridin-2-yl}-N-[(4-methylbenzyl)sulfonyl]piperidine-4-carboxamide
1-{3-cyano-6-[(2-oxopyrrolidin-1-yl)methyl]-5-propionylpyridin-2-yl}-N-[(cyclopentylmethyl)sulfonyl]piperidine-4-carboxamide
1-{3-cyano-5-(cyclopropylcarbonyl)-6-[(2-oxopiperidin-1-yl)methyl]pyridin-2-yl}-N-{[methyl(phenyl)amino]sulfonyl}piperidine-4-carboxamide
1-{3-cyano-5-(cyclopropylcarbonyl)-6-[(2-oxopiperidin-1-yl)methyl]pyridin-2-yl}-N-[(cyclopentylmethyl)sulfonyl]piperidine-4-carboxamide
N-(benzylsulfonyl)-1-{3-cyano-5-isobutyryl-6-[(2-oxopyrrolidin-1-yl)methyl]pyridin-2-yl}piperidine-4-carboxamide
1-{3-cyano-5-isobutyryl-6-[(2-oxopyrrolidin-1-yl)methyl]pyridin-2-yl}-N-[(cyclopentylmethyl)sulfonyl]piperidine-4-carboxamide
1-{3-cyano-6-[(2-oxopyrrolidin-1-yl)methyl]-5-pentanoylpyridin-2-yl}-N-[(cyclopentylmethyl)sulfonyl]piperidine-4-carboxamide
N-(benzylsulfonyl)-1-{3-cyano-6-[(2-oxopyrrolidin-1-yl)methyl]-5-pentanoylpyridin-2-yl}piperidine-4-carboxamide
1-{3-cyano-6-[(2-oxopyrrolidin-1-yl)methyl]-5-pentanoylpyridin-2-yl}-N-[(4-methylbenzyl)sulfonyl]piperidine-4-carboxamide
1-{3-cyano-6-[(2-oxopyrrolidin-1-yl)methyl]-5-pentanoylpyridin-2-yl}-N-[(2,4-difluorobenzyl)sulfonyl]piperidine-4-carboxamide
1-{3-cyano-6-[(2-oxopyrrolidin-1-yl)methyl]-5-pentanoylpyridin-2-yl}-N-{[(4-fluorophenyl)(methyl)amino]sulfonyl}piperidine-4-carboxamide
1-{3-cyano-6-[(2-oxopyrrolidin-1-yl)methyl]-5-pentanoylpyridin-2-yl}-N-{[methyl(phenyl)amino]sulfonyl}piperidine-4-carboxamide
1-{3-cyano-5-(cyclopropylcarbonyl)-6-[(2-oxopyrrolidin-1-yl)methyl]pyridin-2-yl}-N-{[methyl(phenyl)amino]sulfonyl}piperidine-4-carboxamide
1-{3-cyano-5-(cyclopropylcarbonyl)-6-[(2-oxopyrrolidin-1-yl)methyl]pyridin-2-yl}-N-[(cyclobutylmethyl)sulfonyl]piperidine-4-carboxamide
1-{3-cyano-5-(cyclopropylcarbonyl)-6-[(2-oxopyrrolidin-1-yl)methyl]pyridin-2-yl}-N-{[(4-fluorophenyl)(methyl)amino]sulfonyl}piperidine-4-carboxamide
N-(benzylsulfonyl)-1-{3-cyano-6-[(2-oxopyrrolidin-1-yl)methyl]-5-pent-4-enoylpyridin-2-yl}piperidine-4-carboxamide
1-{3-cyano-6-[(2-oxopyrrolidin-1-yl)methyl]-5-pent-4-enoylpyridin-2-yl}-N-[(cyclopentylmethyl)sulfonyl]piperidine-4-carboxamide
1-{3-cyano-6-[(2-oxopyrrolidin-1-yl)methyl]-5-pent-4-enoylpyridin-2-yl}-N-{[(4-fluorophenyl)(methyl)amino]sulfonyl}piperidine-4-carboxamide
1-{3-cyano-6-[(2-oxopyrrolidin-1-yl)methyl]-5-pent-4-enoylpyridin-2-yl}-N-{[methyl(phenyl)amino]sulfonyl}piperidine-4-carboxamide
N-(benzylsulfonyl)-1-{5-butyryl-3-chloro-6-[(2-oxopyrrolidin-1-yl)methyl]pyridin-2-yl}piperidine-4-carboxamide
1-{5-butyryl-3-chloro-6-[(2-oxopyrrolidin-1-yl)methyl]pyridin-2-yl}-N-{[(4-fluorophenyl)(methyl)amino]sulfonyl}piperidine-4-carboxamide
1-{5-butyryl-3-chloro-6-[(2-oxopyrrolidin-1-yl)methyl]pyridin-2-yl}-N-[(4-methylbenzyl)sulfonyl]piperidine-4-carboxamide
1-{5-butyryl-3-cyano-6-[(2-oxopyrrolidin-1-yl)methyl]pyridin-2-yl}-N-{[(4-fluorophenyl)(methyl)amino]sulfonyl}piperidine-4-carboxamide
1-{5-butyryl-3-cyano-6-[(2-oxopyrrolidin-1-yl)methyl]pyridin-2-yl}-N-{[methyl(phenyl)amino]sulfonyl}piperidine-4-carboxamide
1-{5-butyryl-3-cyano-6-[(2-oxopyrrolidin-1-yl)methyl]pyridin-2-yl}-N-[(4-methoxybenzyl)sulfonyl]piperidine-4-carboxamide
1-{5-butyryl-3-cyano-6-[(2-oxopyrrolidin-1-yl)methyl]pyridin-2-yl}-N-[(cyclohexylmethyl)sulfonyl]piperidine-4-carboxamide
1-{5-butyryl-3-cyano-6-[(2-oxopyrrolidin-1-yl)methyl]pyridin-2-yl}-N-{[(4-fluorophenyl)amino]sulfonyl}piperidine-4-carboxamide
N-(anilinosulfonyl)-1-{5-butyryl-3-cyano-6-[(2-oxopyrrolidin-1-yl)methyl]pyridin-2-yl}piperidine-4-carboxamide
1-{5-butyryl-3-chloro-6-[(2-oxopyrrolidin-1-yl)methyl]pyridin-2-yl}-N-[(cyclopentylmethyl)sulfonyl]piperidine-4-carboxamide
1-{3-cyano-6-[(2-oxopyrrolidin-1-yl)methyl]-5-pent-4-enoylpyridin-2-yl}-N-[(4-methylbenzyl)sulfonyl]piperidine-4-carboxamide
1-{3-cyano-6-[(2-oxopyrrolidin-1-yl)methyl]-5-pent-4-enoylpyridin-2-yl}-N-[(2,4-difluorobenzyl)sulfonyl]piperidine-4-carboxamide
1-{3-cyano-6-[(2-oxopyrrolidin-1-yl)methyl]-5-(4,4,4-trifluorobutanoyl)pyridin-2-yl}-N-[(2,4-difluorobenzyl)sulfonyl]piperidine-4-carboxamide
1-{3-cyano-6-[(2-oxopyrrolidin-1-yl)methyl]-5-(4,4,4-trifluorobutanoyl)pyridin-2-yl}-N-[(4-methylbenzyl)sulfonyl]piperidine-4-carboxamide
1-{3-cyano-6-[(2-oxopyrrolidin-1-yl)methyl]-5-(4,4,4-trifluorobutanoyl)pyridin-2-yl}-N-[(cyclopentylmethyl)sulfonyl]piperidine-4-carboxamide
1-{3-cyano-6-[(2-oxopyrrolidin-1-yl)methyl]-5-(4,4,4-trifluorobutanoyl)pyridin-2-yl}-N-{[(4-fluorophenyl)(methyl)amino]sulfonyl}piperidine-4-carboxamide
1-{3-cyano-6-[(2-oxopyrrolidin-1-yl)methyl]-5-pentanoylpyridin-2-yl}-N-{[(4-fluorophenyl)amino]sulfonyl}piperidine-4-carboxamide
N-[(4-cyanobenzyl)sulfonyl]-1-{3-cyano-6-[(2-oxopyrrolidin-1-yl)methyl]-5-pentanoylpyridin-2-yl}piperidine-4-carboxamide
1-{3-cyano-6-[(2-oxopyrrolidin-1-yl)methyl]-5-pentanoylpyridin-2-yl}-N-[(cyclohexylmethyl)sulfonyl]piperidine-4-carboxamide
1-{3-cyano-6-[(2-oxopyrrolidin-1-yl)methyl]-5-pentanoylpyridin-2-yl}-N-[(4-isopropylbenzyl)sulfonyl]piperidine-4-carboxamide
1-{3-chloro-6-[(2-oxopyrrolidin-1-yl)methyl]-5-pent-4-enoylpyridin-2-yl}-N-{[(4-fluorophenyl)(methyl)amino]sulfonyl}piperidine-4-carboxamide
N-(benzylsulfonyl)-1-{3-chloro-6-[(2-oxopyrrolidin-1-yl)methyl]-5-pent-4-enoylpyridin-2-yl}piperidine-4-carboxamide
1-{5-butyryl-3-chloro-6-[(2-oxopyrrolidin-1-yl)methyl]pyridin-2-yl}-N-[(cyclohexylmethyl)sulfonyl]piperidine-4-carboxamide
1-{5-butyryl-3-chloro-6-[(2-oxopyrrolidin-1-yl)methyl]pyridin-2-yl}-N-[(2,4-difluorobenzyl)sulfonyl]piperidine-4-carboxamide
1-{3-chloro-6-[(2-oxopyrrolidin-1-yl)methyl]-5-pentanoylpyridin-2-yl}-N-[(cyclopentylmethyl)sulfonyl]piperidine-4-carboxamide
1-{3-chloro-6-[(2-oxopyrrolidin-1-yl)methyl]-5-pentanoylpyridin-2-yl}-N-[(2,4-difluorobenzyl)sulfonyl]piperidine-4-carboxamide
1-{3-chloro-6-[(2-oxopyrrolidin-1-yl)methyl]-5-pentanoylpyridin-2-yl}-N-{[(4-fluorophenyl)(methyl)amino]sulfonyl}piperidine-4-carboxamide
N-(benzylsulfonyl)-1-{3-chloro-6-[(2-oxopyrrolidin-1-yl)methyl]-5-pentanoylpyridin-2-yl}piperidine-4-carboxamide
N-(benzylsulfonyl)-1-{3-cyano-6-[(2-oxopyrrolidin-1-yl)methyl]-5-(4,4,4-trifluorobutanoyl)pyridin-2-yl}piperidine-4-carboxamide
N-[(4-chlorobenzyl)sulfonyl]-1-{3-chloro-6-[(2-oxopyrrolidin-1-yl)methyl]-5-pentanoylpyridin-2-yl}piperidine-4-carboxamide
1-{3-chloro-6-[(2-oxopyrrolidin-1-yl)methyl]-5-pentanoylpyridin-2-yl}-N-[(1-phenylethyl)sulfonyl]piperidine-4-carboxamide
1-{3-cyano-6-[(2-oxopyrrolidin-1-yl)methyl]-5-pentanoylpyridin-2-yl}-N-[(1-phenylethyl)sulfonyl]piperidine-4-carboxamide
1-{3-cyano-5-(5-ethyl-1,3-oxazol-2-yl)-6-[(2-oxopyrrolidin-1-yl)methyl]pyridin-2-yl}-N-{[(4-fluorophenyl)(methyl)amino]sulfonyl}piperidine-4-carboxamide
N-(benzylsulfonyl)-1-{3-cyano-5-(5-ethyl-1,3-oxazol-2-yl)-6-[(2-oxopyrrolidin-1-yl)methyl]pyridin-2-yl}piperidine-4-carboxamide
1-{3-chloro-5-(5-ethyl-1,3-oxazol-2-yl)-6-[(2-oxopyrrolidin-1-yl)methyl]pyridin-2-yl}-N-{[(4-fluorophenyl)(methyl)amino]sulfonyl}piperidine-4-carboxamide
N-(benzylsulfonyl)-1-{3-chloro-5-(5-ethyl-1,3-oxazol-2-yl)-6-[(2-oxopyrrolidin-1-yl)methyl]pyridin-2-yl}piperidine-4-carboxamide
N-(benzylsulfonyl)-1-{5-butyryl-3-chloro-6-[(2-oxopiperidin-1-yl)methyl]pyridin-2-yl}piperidine-4-carboxamide
1-{5-butyryl-3-chloro-6-[(2-oxopiperidin-1-yl)methyl]pyridin-2-yl}-N-{[(4-fluorophenyl)(methyl)amino]sulfonyl}piperidine-4-carboxamide
1-{5-butyryl-3-cyano-6-[(2-oxopyrrolidin-1-yl)methyl]pyridin-2-yl}-N-[(1-phenylethyl)sulfonyl]piperidine-4-carboxamide
1-{5-butyryl-3-chloro-6-[(2-oxopyrrolidin-1-yl)methyl]pyridin-2-yl}-N-{[(4-fluorophenyl)(methyl)amino]sulfonyl}-N-methylpiperidine-4-carboxamide
1-{5-butyryl-3-cyano-6-[(2-oxopyrrolidin-1-yl)methyl]pyridin-2-yl}-N-[(cyclopentylmethyl)sulfonyl]-N-methylpiperidine-4-carboxamide
1-{3-chloro-6-[(2-oxopyrrolidin-1-yl)methyl]-5-pentanoylpyridin-2-yl}-N-[(2,4-difluorobenzyl)sulfonyl]-N-methylpiperidine-4-carboxamide
N-(benzylsulfonyl)-1-{5-butyryl-3-fluoro-6-[(2-oxopyrrolidin-1-yl)methyl]pyridin-2-yl}piperidine-4-carboxamide;

and pharmaceutically acceptable salts thereof.

Processes

The following processes together with the intermediates are provided as a further feature of the present invention.

Compounds of formula (I) may be prepared by the following processes a1-a12;

a1) Compounds of formula (I) in which R₁, R₂, R₃, R₄, B, R₅, R₁₄, R₁₅, R^{c} and R^{d} are defined as in formula (I) above, X is a single bond or a carbon, can be formed by reacting a compound of formula (II), in which R₁, R₂, R₃, R₄, B, R₁₄, and R₁₅ are defined

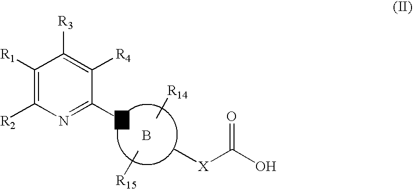

as in formula (I) above, X is a single bond or a carbon, with a compound of formula (III) in which R₅, R^{c} and R^{d} are defined as in formula (I) above.

R₅—NHSO₂—R^{c}—R^{d} (III)

The reaction is generally carried out in an inert organic solvent such as dichloromethane at ambient temperature. The reaction may be carried out using standard conditions or in the presence of TBTU, EDCI, PyBrop or the combination of EDCI and HOBt. Optionally, the reaction may be carried out in the presence of an organic base such as triethylamine or DIPEA.

a2) Compounds of formula (I) in which R₁, R₂, R₃, R₄, B, R₅, R₁₄, R₁₅, R^{c} and R^{d} are defined as in formula (I) above, X is a nitrogen, (—CH₂—NH—) or a single bond connected to a nitrogen which is a member of the B ring, can be formed by reacting a compound of formula (IV), in which R₁, R₂, R₃, R₄, B, R₁₄, and R₁₅ are defined as in formula (I) above and X is a nitrogen, (—CH₂—NH₂) or a hydrogen that is connected to a nitrogen which is a member of the B-ring, with a compound of the general

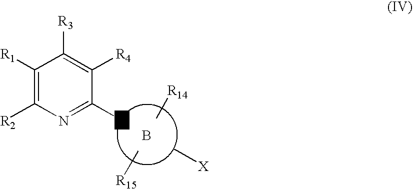

formula (III) which is defined as above.

The reaction is generally carried out in an inert solvent such as DCM. The reaction may be carried out in the presence of CDI. Optionally, the reaction may be carried out in the presence of an organic base such as triethylamine, DBU or DIPEA.

a3) Compounds of formula (I) in which R₁, R₂, R₃, R₄, B, R₁₄, R₁₅, R^{c} and R^{d} are defined as in formula (I) above, R₅ is a hydrogen, X is a nitrogen, (—CH₂—NH—) or a single bond connected to a nitrogen which is a member of the B ring, can be formed by reacting a compound of formula (IV) which is defined in a2) above, with a compound of formula (V)

O═C═N—SO₂—R^{c}R^{d} (V)

in which R^{c} and R^{d} are defined as in formula (I) above.

The reaction is generally carried out in an inert solvent such as THF. Optionally, the reaction may be carried out in the presence of an organic base such as triethylamine or DIPEA.

a4) Compounds of formula (I) in which R₁, R₂, R₃, R₄, B, R₅, R₁₄, R₁₅, R^{c} and R^{d} are defined as in formula (I) above, X is a nitrogen, (—CH₂—NH—) or a single bond connected to a nitrogen which is a member of the B ring, can be formed by reacting a compound of formula (IV) which is defined in above, with a compound of formula (VI),

R^{d}R^{c}—SO₂NR₅—COOCH₂CCl₃ (VI)

in which R₅, R^{c} and R^{d} are defined as in formula (I) above. The reaction is generally carried out in an inert solvent such as DMA. Optionally, the reaction may be carried out in the presence of an organic base such as triethylamine or DIPEA.

a5) Compounds of formula (I) may also be prepared by reacting a compound of formula (VII) in which R₁, R₂, R₃, and R₄ are defined as in formula (I) above and L is a suitable leaving group, such as chloro, bromo, iodo, fluoro, triflate (OTf) mesylate (OMs) or tosylate (OTs),

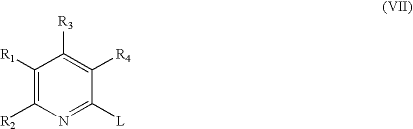

with a compound of the general formula (VIII) in which B, X, R₅, R₁₄, R₁₅, R^{c} and R^{d} are defined as in formula (I) above.

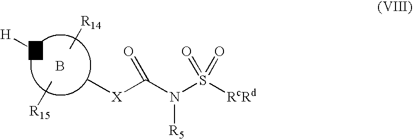

The reaction is generally carried out in an inert solvent such as DMA. Optionally, the reaction may be carried out in the presence of an organic base such as triethylamine or DIPEA.

The reaction is generally carried out at elevated temperatures using standard equipment or in a single-node microwave oven.

For some compounds, it is advantageous to carry out the reaction in ethanol in the presence of an organic base such as triethylamine.

a6) Compounds of formula (I) where R₁ represents R₆OC(O) and R₂, R₃, R₄, B, R₅, R₆, R₁₄, R₁₅, X, R^{c} and R^{d} are defined as in formula (I) above, can be transesterified using standard procedures or by reacting with R_{6′}—O⁻Li⁺ reagent, to become another compound of the general formula (I) wherein R₁ becomes R_{6′}OC(O).

a7) A compound of formula (I) in which R₁, R₂, R₃, R₄, B, R₅, R₁₄, R₁₅, and R^{d} are defined as in formula (I) above and R^{c} represents imino (—NH—) or (C₁-C₄)alkylimino in which the imino group could be substituted using standard conditions or using an alkylating agent like L-R₁₉, in which R₁₉ is defined as in formula (I) above and L is a leaving group exemplified by chloro, bromo, iodo, triflate(OTf) or tosylate(OTs), to give compounds of formula (I) in which R₁, R₂, R₃, R₄, B, R₅, R₁₄, R₁₅, and R^{d} are defined as in formula (I) above and R^{c} represents N-substituted imino (—NR₁₉—) or N-substituted (C₁-C₄)alkylimino (—N(R₁₉)—((C₁-C₄)alkyl), optionally in the presence of a strong base such as NaH.

a8) Compounds of formula (I) in which R₁ is R₆OC(O) and R₃, R₄, B, R₅, R₆, R₁₄, R₁₅, X, R^{c} and R^{d} are as defined in formula (I) above, R₂ is a substituted (C₁-C₁₂)alkoxy group defined as in formula (I) above may be prepared by reacting a compound of formula (IX)

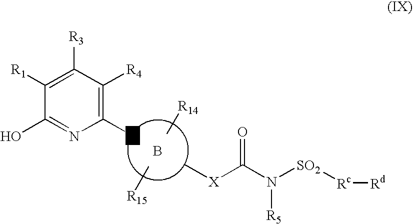

in which R₁ is R₆OC(O) and R₃, R₄, B, R₅, R₆, R₁₄, R₁₅, X, R^{c} and R^{d} are as defined in formula (I) above with a compound of formula (X)

L-R_{2′} (X)

in which R₂′ is a substituted (C₁-C₁₂)alkyl defined as in formula (I) above and L is a leaving group such as chloro, bromo, iodo, triflate (OTf) or tosylate (OTs).

The reaction may be carried out in an inert organic solvent such as DMA, THF or CH₃CN. The reaction may be carried out using standard conditions or in the presence of a suitable base such as sodium hydride, DIPEA or silver carbonate or potassium carbonate. Preferentially silvercarbonate is used.

The reaction may be carried out at ambient temperature or at elevated temperatures using standard equipment or a single node microwave oven.

a9) Compounds of formula (I) in which R₁ is R₆OC(O) and R₃, R₄, B, R₅, R₆, R₁₄, R₁₅, X, R^{c} and R^{d} are as defined in formula (I) above, R₂ is a (C₁-C₁₂)alkylcarbonyloxy, arylcarbonyloxy or heterocyclylcarbonyloxy group defined as above can be prepared by reacting a compound of formula (IX) defined as above with the corresponding carboxylic acid chloride or a carboxylic acid anhydride.

The reaction may be carried out in an inert organic solvent such as DCM or THF. The reaction may be carried out using standard conditions or in the presence of a suitable base such as DIPEA, Pyridine or DMAP.

The reaction may be carried out at ambient temperature or at elevated temperatures using standard equipment or a single node microwave oven.

a10) Compounds of formula (I) in which R₁ is R₆OC(O) and R₃, R₄, B, R₅, R₆, R₁₄, R₁₅, X, R^{c} and R^{d} are as defined in formula (I) above, R₂ is a substituted (C₁-C₁₂)alkoxy group or a substituted (C₁-C₁₂)alkylthio group defined as in formula (I) above can be prepared by reacting a compound of formula (XI)

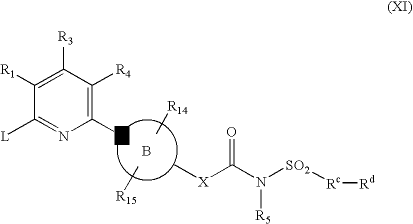

in which R₁ is R₆OC(O) and R₃, R₄, B, R₅, R₆, R₁₄, R₁₅, X, R^{c} and R^{d} are as defined in formula (I) above and L is a suitable leaving group such as Cl, Br, I or triflate (OTf) with the corresponding substituted (C₁-C₁₂)alcohol and substituted (C₁-C₁₂)alkylthiol respectively.

The reaction may be performed using standard conditions in the presence of a palladium catalyst such as or Pd(PPh₃)₄ or Pd₂(dba)₃ in combination with a suitable phosphine ligand such as PPh₃ or XANTPHOS. The reaction may be carried out in an inert solvent such as DCM, THF or dioxane optionally in the presence of a base such as DIPEA.

The reaction may be carried out at ambient temperature or at elevated temperatures using standard equipment or a single node microwave oven.

a11) Compounds of formula (I) in which R₁ is R₆OC(O) and R₃, R₄, B, R₅, R₆, R₁₄, R₁₅, X, R^{c} and R^{d} are as defined in formula (I) above, R₂ is a substituted C₁-alkyl group defined as in formula (I) above can be prepared by reacting a compound of formula (XII)

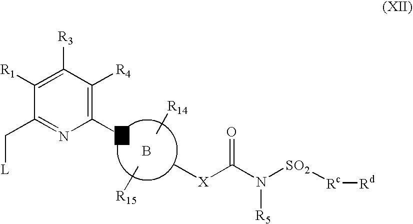

in which R₁ is R₆OC(O) and R₃, R₄, B, R₅, R₆, R₁₄, R₁₅, X, R^{c} and R^{d} are as defined in formula (I) above and L is a suitable leaving group such as Cl, Br, I, triflate (OTf) or tosylate (OTs) with the corresponding nucleophile to give the substituted C₁-alkyl group described for R₂ above.

The reaction is carried out using standard conditions in an inert solvent such as EtOH, DMF or acetone.

Preferentially the reaction is carried out in the presence of a base such as DIPEA, TEA or Cs₂CO₃.

Optionally the reaction is performed in the presence of sodium iodide.

The reaction may be carried out at ambient temperature or at elevated temperatures using standard equipment or a single node microwave oven.

a12) Compounds of formula (I) in which R₁, R₂, R₃, R₄, X, B, R₁₄, R₁₅, R^{c} and R^{d} are defined as in formula (I) above and R₅ is (C₁-C₁₂)alkyl or carboxy(C₁-C₆)alkyl may be prepared by reaction of a compound of formula (I) in which R₁, R₂, R₃, R₄, X, B, R₁₄, R₁₅, R^{c} and R^{d} are defined as in formula (I) and R₅ is H with a compound of formula (C₁-C₁₂)alkyl-L or carboxy(C₁-C₆)alkyl-L respectively, wherein L is a leaving group such as Cl, Br, I, triflate (OTf) os tosylate (OTs).

The reaction is carried out in an inert organic solvent such as DMF, THF or CH₃CN. The reaction may be carried out using standard conditions or in the presence of a suitable base such as sodium hydride, DIPEA or silver carbonate or potassium carbonate.

The reaction may be carried out at ambient temperature or at elevated temperatures using standard equipment or a single node microwave oven.

a13) Compounds of formula (I) may also be prepared by reacting a compound of formula (VII) in which R₁, R₂, R₃, and R₄ are defined as in formula (I) above except that L is a hydroxy group with a compound of the general formula (VIII) in which B, X, R₅, R₁₄, R₁₅, R^{c} and R^{d} are defined as in formula (I) above.

The reaction is generally carried out in an inert organic solvent such as DCM or THF at ambient temperature. The reaction is carried out in the presence of a suitable coupling reagent such as for example PyBrop preferentially in the presence of an organic base such as TEA or DIPEA.

The intermediates referred to above may be prepared by, for example, the methods/processes outlined below.

b1) The compounds of formula (II) in which R₁, R₂, R₃, R₄, B, R₁₄, and R₁₅ are defined as in formula (I) above, X is a single bond or a carbon, may be prepared by reacting a compound of formula (VII) defined above and L is a suitable leaving group (such as fluoro, chloro, bromo, iodo, triflate (OTf) mesylate (OMs) or tosylate (OTs)), with a compound of the general formula (XIII),

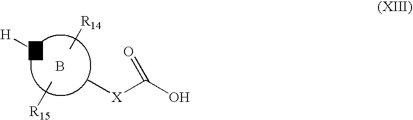

in which B, R₁₄, R₁₅ are defined as in formula (I) above and X is a single bond or a carbon.

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the presence of an organic base such as TEA or DIPEA.

b2) The compounds of formula (II) in which R₁, R₂, R₃, R₄, B, R₁₄, and R₁₅ are defined as in formula (I) above, X is a single bond or a carbon, may be prepared by reacting a compound of formula (VII) defined above except that L is a hydroxy group with a compound of the general formula (XIII) in which B, X, R₅, R₁₄, and R₁₅ are defined as in formula (I) above.

The reaction is generally carried out in an inert organic solvent such as DCM or THF at ambient temperature. The reaction is carried out in the presence of a suitable coupling reagent such as for example PyBrop preferentially in the presence of an organic base such as TEA or DIPEA.
c1) Compounds of formula (IV) which are defined as above may be prepared by reacting the corresponding compound of formula (VII) which is defined above, with a compound of formula (XIV) in which B, R₁₄, R₁₅ are defined as in formula (I) above, X is a nitrogen, (—CH₂—NH—) or a single bond connected to a nitrogen which is a member of the B ring.

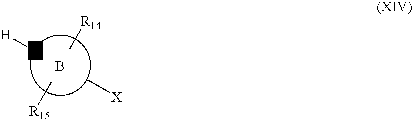

The reaction is generally carried out at elevated temperatures using standard equipment or in a single-node microwave oven. The reaction can be carried out in an inert solvent such as ethanol, DMA or a mixture of solvents such as ethanol-water. Optionally the reaction may be carried out in the presence of an organic base such as TEA or DIPEA.

c2) Compounds of formula (IV) which are defined as above may be prepared by reacting the corresponding compound of formula (VII) which is defined above except that L is a hydroxy group, with a compound of formula (XIV) in which B, R₁₄, R₁₅ are defined as in formula (I) above, X is a nitrogen, (—CH₂—NH—) or a single bond connected to a nitrogen which is a member of the B ring.

The reaction is generally carried out in an inert organic solvent such as DCM or THF at ambient temperature. The reaction is carried out in the presence of a suitable coupling reagent such as for example PyBrop preferentially in the presence of an organic base such as TEA or DIPEA.

d) Synthesis of compounds of the general formula (XV),

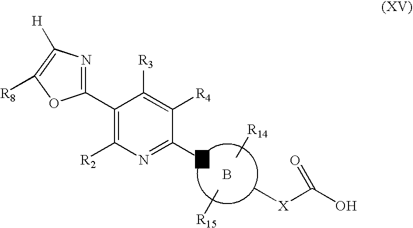

in which R₂, R₃, R₄, B, R₈, R₁₄ and R₁₅ are defined as in formula (I) above and X is a carbon or a single bond comprises the below steps. (d1-d5)

d1) Reacting the corresponding compounds of the general formula (XIII) which is defined as above with a compound of the general formula (XVI)

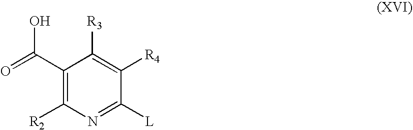

in which R₂, R₃ and R₄ are defined as in formula (I) above, and L is a suitable leaving group, such as chloro, bromo, iodo, triflate (OTf), mesylate (OMs) or tosylate (OTs), to give a compound of formula (XVII).

The reactions are carried out at elevated temperatures using standard equipment or a single-node microwave oven. Optionally the reaction may be carried out in the presence of an organic base such as TEA or DIPEA.

d2) The compounds of formula (XVII) can then be reacted

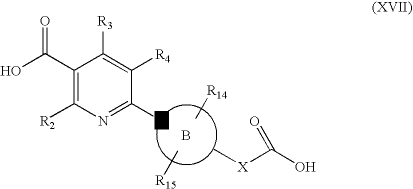

with a compound of the general formula (XVIII),

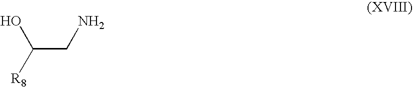

in which R₈ is defined as in formula (I) above, to give compounds of the general formula (XIX). The reactions may be carried out using standard conditions or in the presence of EDCI or the combination of EDCI and HOBt. Optionally the reaction may be carried out in the presence of an organic base such as TEA or DIPEA.

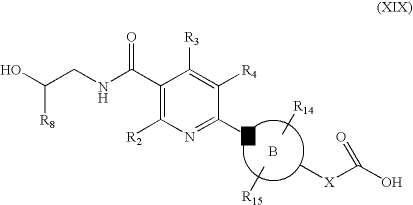

d3) This compound (XIX) can then be transformed to a compound of the general formula (XX)

d4) The preparation of compounds with the general formula (XX),

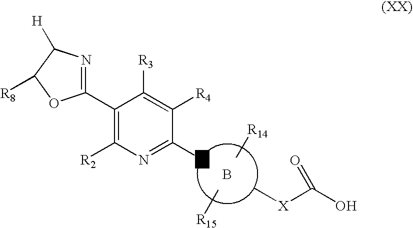

in which R₂, R₃, R₄, B, R₈, R₁₄ and R₁₅ are defined as in formula (I) above and X is a carbon or a single bond using known methods or a known reagent such as methanesulfonyl chloride. Optionally the reaction may be carried out in the presence of an organic base such as TEA.

d5) a compound of the general formula (XV) as defined above can be made by oxidizing the corresponding compound of the general formula (XX) using a known oxidation reagent such as DDQ.

e) The preparation of compounds of the general formula (XV) also comprises the steps (e1-e7) below;

e1) Reacting a compound the general formula (XXI),

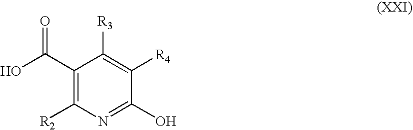

in which R₂, R₃ and R₄ are defined as in formula (I) above, with a compound of the general formula (XXII), in which R₈ is defined as in formula (I) above,

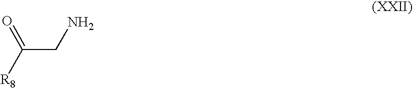

using standard conditions or in the presence of EDCI or the combination of EDCI and HOBt. Optionally the reaction may be carried out in the presence of an organic base such as TEA. This reaction gives a compound of the general formula (XXIII).

e2) The compound of the general formula (XXIII) obtained

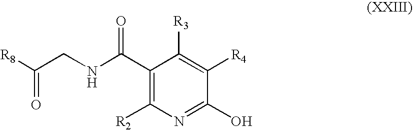

can then be transformed to a compound of the general formula (XXIV), in which R₂, R₃, R₄ and R₈ are defined as in formula (I) above, using known techniques or using a known reagent such as POCl₃ or in the presence of (Methoxycarbonylsulfamoyl)triethylammonium hydroxide (Burgess reagent).

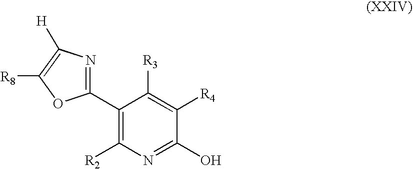

The preparation of compounds of the general formula (XXIV) which is defined as above can also comprise the steps (e3-e5) below;

e3) Reacting a compound of the general formula (XXI) above with a compound of the general formula (XVIII), defined as above, to give a compound of the formula (XXV). The reaction is generally carried out in DCM at ambient temperature. The reaction may be carried out using standard conditions or in the presence of EDCI or the combination of EDCI and HOBt. Optionally the reaction may be carried out in the presence of an organic base such as TEA or DIPEA.

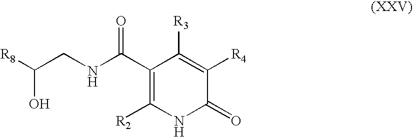

e4) The compound of formula (XXV) can be transformed to a compound (XXIII) using standard conditions or an oxidizing agent such as the mixture of oxalylchloride and DMSO.

e5) The compound of formula (XXIII) can then be transformed into a compound of the general formula (XXIV), using standard conditions or in the presence of (Methoxycarbonylsulfamoyl)triethylammonium hydroxide (Burgess reagent). The reaction is generally performed in an inert solvent such as THF. The reaction is carried out at elevated temperatures using standard equipment or a single-node microwave oven.

e6) A compound of the general formula (XXIV) can then be transformed to a compound of the general formula (XXVI),

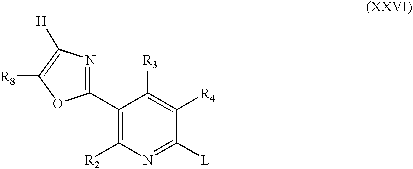

in which R₂, R₃, R₄, R₈ are defined as in formula (I) above and L is a sufficient leaving group, such as chloro, bromo, iodo, triflate (OTf), mesylate (OMs) or tosylate (OTs), using a known techniques or a reagent such as oxalyl chloride or thionyl chloride.

e7) The compound of formula (XXVI) can then be reacted with a compound of the general formula (XIII), which is defined as above, to give a compound of the general formula (XV), defined as above. The reactions are carried out at elevated temperatures using standard equipment or a single-node microwave oven. Optionally the reactions may be carried out in the presence of an organic base such as TEA or DIPEA.

f) Preparation of Compounds of the general formula (XXVII),

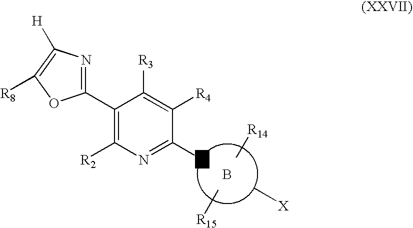

in which R₂, R₃, R₄, B, R₈, R₁₄ and R₁₅ are defined as in formula (I) above, X is a nitrogen, (—CH₂—NH—) or a single bond connected to a nitrogen which is a member of the B ring, comprises the below steps. (f1-f4)

f1) Reacting a compound of the general formula (XIV) which is defined as above with a compound of the general formula (XVI) which is defined as above, to give a compound of the general formula (XXVIII).

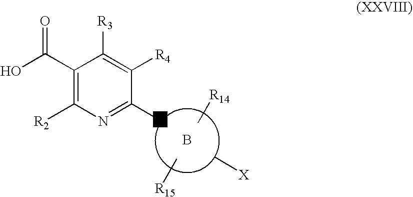

The reactions are carried out at elevated temperatures using standard equipment or a single-node microwave oven. Optionally the reaction may be carried out in the prescence of an organic base such as TEA or DIPEA.

f2) The compound of formula (XXVIII) can be reacted with a compound of formula (XVIII), which is defined as above, to give compounds of the general formula (XXIX). The reactions are carried out using standard conditions or in the presence of EDCI or the combination of EDCI and HOBt. Optionally the reactions may be carried out in the presence of an organic base such as TEA or DIPEA.

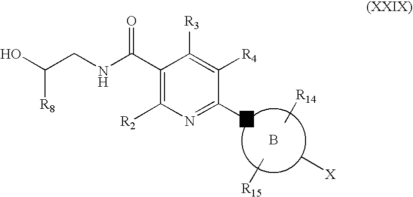

f3) This compound can then be transformed to a compound of the general formula (XXX) in which R₂, R₃, R₄, B, R₈, R₁₄ and R₁₅, are defined as in formula (I) above,

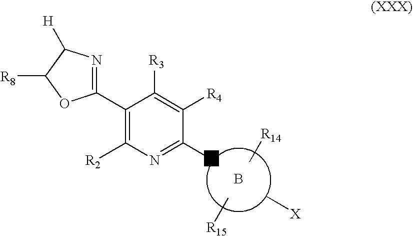

X is a nitrogen, (—CH₂—NH—) or a hydrogen connected to a nitrogen which is a member of the B ring, using known methods or a sufficient reagent such as methanesulfonyl chloride. Optionally the reaction may be carried out in the presence of an organic base such as TEA.

f4) (XXVII) can then prepared by oxidizing a compound of the general formula (XXX), which is defined as above. The reaction can be performed using standard conditions or a reagent like DDQ.

Compounds of the general formula (I), in which R₁ is R₇C(O) and R₂, R₃, R₄, R₇, B, R₁₄ and R₁₅ are defined as in formula (I) above, X is a single bond or a carbon atom comprises the following steps (g1)-g2):

g1) Reacting a compound of the general formula (XVII), described above, with N,O-dimethylhydroxylamine. The reaction can be performed using known reagents like CDI, EDCI or the combination of EDCI and HOBt to give a compound of the general formula (XXXI)

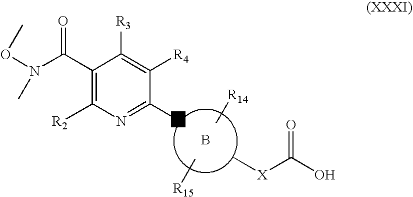

g2) Reacting compounds of the general formula (XXXI), defined as above, with a reagent of the general formula R₇—MgX′, in which R₇ is defined as in formula (I) above and X′ is a halogen, or a reagent of the formula R₇-M, in which M is a metal examplified by Zn and Li.

Compounds of the general formula (II), in which R₁ is R₇C(O) and R₂, R₃, R₄, R₇, B, R₁₄ and R₁₅ are defined as in formula (I) above, X is a single bond or a carbon atom also comprises the following steps (g3-g4):

g3) Reacting compounds of general formula LI

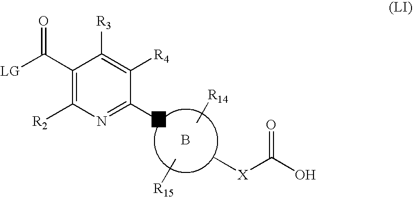

wherein R₂, R₃, R₄, B, R₁₄ and R₁₅ is as defined in formula (I) above, X is a single bond or a carbon atom and LG is a leaving group such as Cl or F with a reagent of general formula R₇—MgX′, in which R₇ is defined as in formula (I) above.

The reaction is carried out using standard conditions in an inert solvent such as THF catalyzed by ferric acetylacetonate or other suitable ferric salts such as for example FeCl₃.

The reaction may be performed at ambient temperature or preferentially at lower temperatures for example in the range of −78° C. and 0° C.

(See for example Fürstner A et al, J. Org Chem, 2004, pp 3943-3949)

g4) Compounds of general formula (LI) above can by prepared by reacting a compound of general formula (XVII) defined as above using standard conditions or with a chlorinating reagent such as oxalyl chloride, thionyl chloride or POCl₃ (e.g. when LG is Cl). Advantageously dimethylformamide may be used as catalyst.

The reaction can also be performed using standard conditions with cyanuric fluoride preferentially in the presence of pyridine (e.g. when LG is F)

The reaction may be performed in an inert solvent such as DCM or toluene. The reaction is carried out at ambient temperature or at elevated temperatures.

Compounds of the general formula (II), in which R₁ is R₇C(O) (this is a special case for all compounds which contains a R₇ group containing a CH₂ group next to the cabonyl in R₁ referred to below as R_{7′}—CH₂) and R₂, R₃, R₄, R₇, B, R₁₄ and R₁₅ are defined as in formula (I) above, X is a single bond or a carbon atom also comprises the following steps (g5-g7)

g5) By double decarboxylation of a compound of general formula (LII)

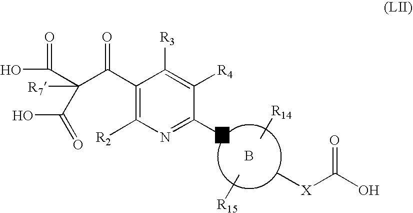

The reaction is generally carried at elevated temperature using standard equipment. Preferentially the reaction is carried out under acidic conditions in an inert solvent such as MeCN or THF.

g6) Compounds of the formula (LII) above can be prepared by reaction of a compound of formula (LI) with a compound of formula (LIII)

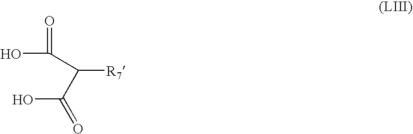

The reaction is carried out in an inert solvent such as THF at ambient temperature in the presence of a suitable base such as sodium pentoxide or NaH.

(For similar chemistry see, Asish D. et al, J. Chem. Soc. Perkin Treans. I, 1989, pp 603-607 and Rathke, M et al, J. Org. Chem. 1985, pp 2622-24).

Compounds of the general formula (IV), in which R₁ is R₇C(O) and R₂, R₃, R₄, R₇, B, R₁₄ and R₁₅ are defined as in formula (I) above, X is a nitrogen, (—CH₂—NH—) or a single bond connected to a nitrogen which is a member of the B ring, comprises the following steps (h1-h2).

h1) Reacting a compound of the general formula (XXVIII), defined as above, with N,O-dimethylhydroxylamine. The reaction can be performed using known reagents like CDI, EDCI or the combination of EDCI and HOBt to give a compound of the general formula (XXXII).

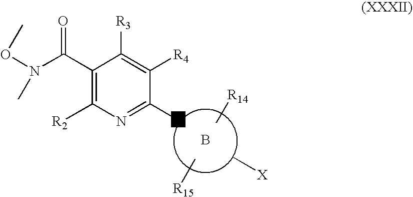

h2) A compound of the general formula (XXXII), which is defined as above can be reacted with a reagent of the general formula R₇—MgX, in which R₇ is defined as in formula (I) above and X is a halogen, or a reagent of the formula R₇-M, in which M is a metal exemplified by Zn and Li.

Compounds of the general formula (IV), in which R₁ is R₇C(O) and R₂, R₃, R₄, R₇, B, R₁₄ and R₁₅ are defined as in formula (I) above, X is a nitrogen, (—CH₂—NH—) or a single bond connected to a nitrogen which is a member of the B ring, also comprises the following steps (h3-h4).

h3) Reacting compounds of general formula LIV

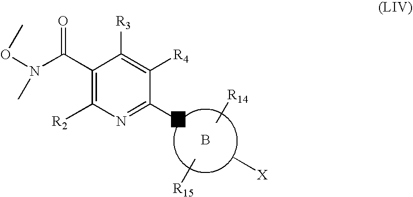

wherein R₂, R₃, R₄, B, R₁₄ and R₁₅ is as defined in formula (I) above, X is a nitrogen, (—CH₂—NH—) or a single bond connected to a nitrogen which is a member of the B ring and LG is a leaving group such as Cl or F with a reagent of general formula R₇—MgX′, in which R₇ is defined as in formula (I) above.

The reaction is carried out using standard conditions in an inert solvent such as THF catalyzed by ferric acetylacetonate or other suitable ferric salts.

The reaction may be performed at ambient temperature or preferentially at lower temperatures for example in the range of −78° C. and 0° C.

(See for example Fürstner A et al, J. Org Chem, 2004, pp 3943-3949)

h4) Compounds of general formula (LIV) above can by prepared by reacting a compound of general formula (XXVIII) defined as above using standard conditions or with a chlorinating reagent such as oxalyl chloride, thionyl chloride or POCl₃ (e.g. when LG is Cl). Advantageously dimethylformamide may be used as catalyst. The reaction can also be performed using standard conditions with cyanuric fluoride preferentially in the presence of pyridine (e.g. when LG is F)

The reaction may be performed in an inert solvent such as DCM or toluene. The reaction is carried out at ambient temperature or at elevated temperatures.

Compounds of the general formula (IV), in which R₁ is R₇C(O) (this is a special case for all compounds which contains a R₇ group containing a CH₂ group next to the cabonyl in R₁ referred to below as R₇, —CH₂) and R₂, R₃, R₄, B, R₁₄ and R₁₅ are defined as in formula (I) above, X is a nitrogen, (—CH₂—NH—) or a single bond connected to a nitrogen which is a member of the B ring, also comprises the following steps (h5-h6).

h5) By double decarboxylation of a compound of general formula (LV)

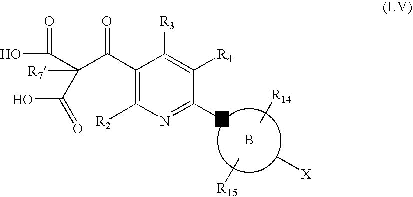

The reaction is generally carried at elevated temperature using standard equipment. Preferentially the reaction is carried out under acidic conditions in an inert solvent such as MeCN or THF.

h6) Compounds of the formula (LV) above can be prepared by reaction of a compound of formula (LIV) with a compound of formula (LIII)

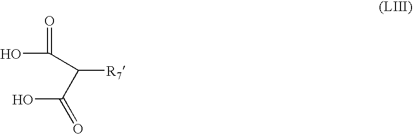

The reaction is carried out in an inert solvent such as THF at ambient temperature in the presence of a suitable base such as sodium pentoxide or NaH.

(For similar chemistry see, Asish D. et al, J. Chem. Soc. Perkin Treans. I, 1989, pp 603-607 and Rathke, M et al, J. Org. Chem. 1985, pp 2622-24).

Compounds of the general formula (VIII) can be formed in one of the processes (i1-i4). The compounds of formula (VIII) in which R₅ is a hydrogen are advantageously isolated as a zwitterion. A ring nitrogen of compounds of formula (XIII) and (XIV) used in the below steps may be protected by a protective group such as t-butyloxycarbonyl.

i1) Compounds of the general formula (VIII) in which B, R₅, R₁₄, R₁₅, R^{c} and R^{d} are defined as in formula (I) above, X is a single bond or a carbon, may be formed by reacting a compound of formula (XIII) with a compound of formula (III). The reaction is generally carried out in an inert organic solvent such as dichloromethane at ambient temperature. The reaction may be carried out using standard conditions or in the presence of EDCI or the combination of EDCI and HOBt. Optionally, the reaction may be carried out in the presence of an organic base such as triethylamine or DIPEA.

i2) Compounds of the general formula (VIII) in which R₅ is hydrogen, B, R₁₄, R₁₅, R^{c} and R^{d} are defined as in formula (I) above, X is a nitrogen, (—CH₂—NH—) or a single bond connected to a nitrogen which is a member of the B ring, can be formed by reacting a compound of formula (XIV) defined as above with a compound of formula (V), defined as above. The reaction is generally carried out in an inert solvent such as THF. The reaction may also be carried out in the presence of an organic base such as triethylamine or DIPEA.

i3) Compounds of the general formula (VIII) in which B, R₅, R₁₄, R₁₅, R^{c} and R^{d} defined as in formula (I) above, X is a nitrogen, (—CH₂—NH—) or a single bond connected to a nitrogen which is a member of the B ring, can also be formed by reacting a compound of formula (XIV) with a compound of formula (VI) which is defined as above. The reaction is generally carried out in a solvent such as DMA. This reaction may also be carried out in the presence of an organic base such as triethylamine or DIPEA

i4) A compound of formula (VIII) which is protected with t-butoxy carbonyl may be transformed into a compound without the protective group using standard procedures or a reagent such as HCl or TFA.

(j) Compounds of the general formula (VII) which are defined as above can be formed by reacting a compound of formula (XXXIII) using standard conditions or with a chlorinating reagent such as oxalyl chloride, thionyl chloride or POCl₃. Advantageously dimethylformamide may be used. The reaction may be performed in an inert solvent such as DCM. Advantageously the inert solvent is toluene.

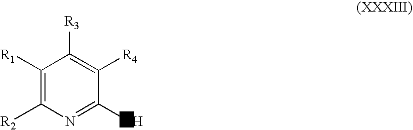

l) Preparation of compounds of the general formula (XXI) which is defined as above except for R₃ which is hydrogen, comprises the following steps (l₁-l₃);

l1) Reacting a compound of the formula (XXXIV), in which R₂ and R₆ are defined as in formula (I) above with dimethoxy-N,N-dimethylmethaneamine to form a

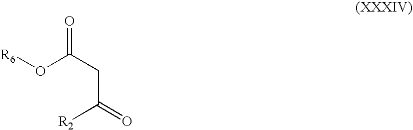

compound of formula (XXXV).

l2) This compound (XXXV) can then be reacted further with a compound of the

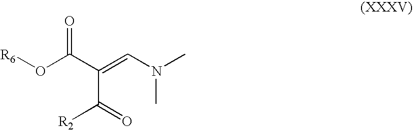

general formula R₄CH₂C(O)NH₂, in which R₄ is defined as in formula (I) above to give a compound of the general formula (XXXVI). The reaction is generally performed in an inert solvent such as ethanol, optionally in the presence of a strong base such as sodium ethoxide.

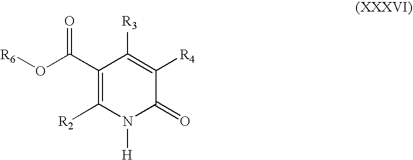

(l3) A compound of the general formula (XXXVI) can then be transformed to a compound of the general formula (XXI). The reaction is generally performed in a protic solvent such as water together with a co-solvent such as THF or methanol. The reaction can be performed using standard reagents or in the presence of LiOH, NaOH or KOH.

m) Compounds of the general formula (IX) wherein R₃, R₁₄, R₁₅, B, X, R₅, R^{c} and R^{d} are defined as in formula (I) R₁ is R₆OC(O) and R₄ is CN may be prepared by the following steps m1-m9 below

m1) Reacting a compound of the general formula (XXXVII)

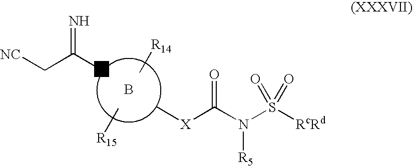

where R₅, B, R₁₄, R₁₅, X, R^{c} and R^{d} are as defined in formula (I) above with a compound of formula (XXXVIII)

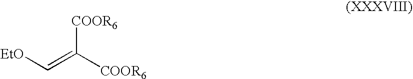

The reaction is generally carried out in an inert organic solvent such as EtOH or DMSO.

The reaction is carried out at ambient temperature or at elevated temperatures using standard equipment or a single node microwave oven.

m2) Compounds of the general formula (XXXVIII) defined above can be prepared by reacting a compound of the general formula (VIII) as defined above with a compound of formula (XXXIX)

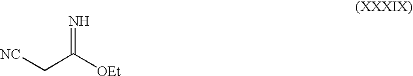

using essentially the same procedure as described in [Macconi, A et. Al., J. Heterocyclic chemistry, 26, p. 1859 (1989)].

m3) Compounds of general formula (IX) above wherein R₃, B, R₁₄, R₁₅, R₅, R^{c} and R^{d} are defined as in formula (I), R₁ is R₆OC(O), R₄ is CN and X is a single bond or a carbon atom may be prepared by reacting a compound of formula (XXXX)

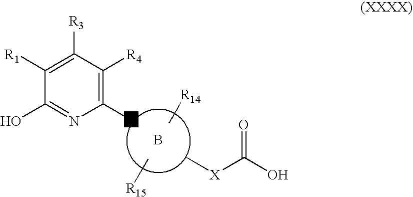

with a compound of formula (III) defined as above.

The reaction is generally carried out in an inert organic solvent such as dichloromethane at ambient temperature. The reaction may be carried out using standard conditions or in the presence of TBTU, EDCI, PyBrop or the combination of EDCI and HOBt. Optionally, the reaction may be carried out in the presence of an organic base such as triethylamine or DIPEA.

m4) Compounds of general formula (XXXX) may be prepared by reacting a compound of general formula (XXXXI)

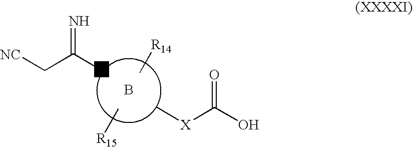

wherein R₁₄, R₁₅, and B is defined as in formula (I) and X is a single bond or a carbon atom with a compound of formula (XXXVIII) defined as above.

The reaction is generally carried out in an inert organic solvent such as EtOH or DMSO.

The reaction is carried out at ambient temperature or at elevated temperatures using standard equipment or a single node microwave oven.

m5) Compounds of the general formula (XXXXI) defined above can be prepared by reacting a compound of the general formula (XIII) as defined above with a compound of formula (XXXIX) using essentially the same procedure as described in [Macconi, A et. Al., J. Heterocyclic chemistry, 26, p. 1859 (1989)].

m6) Compounds of general formula (IX) above wherein R₃, B, R₁₄, R₁₅, R₅, R^{c} and R^{d} are defined as in formula (I), R₁ is R₆OC(O), R₄ is CN and X is a nitrogen, (—CH₂—NH—) or a single bond connected to a nitrogen which is a member of the B ring may be prepared by reacting a compound of formula (XXXXII)

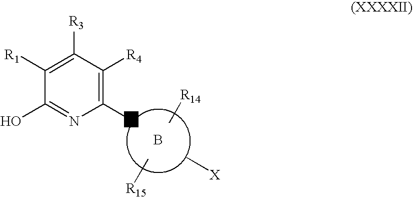

with a compound of formula (III) defined as above.

The reaction is generally carried out in an inert solvent such as DCM. The reaction may be carried out in the presence of CDI. Optionally, the reaction may be carried out in the presence of an organic base such as triethylamine, DBU or DIPEA.

m7) Compounds of general formula (IX) above wherein R₃, R₁₄, R₁₅, R^{c} and R^{d} are defined as in formula (I), R₁ is R₆OC(O), R₄ is CN, R₅ is hydrogen and X is a nitrogen, (—CH₂—NH—) or a single bond connected to a nitrogen which is a member of the B ring may be prepared by reacting a compound of formula (XXXXII) with a compound of general formula (V) as defined above.

The reaction is generally carried out in an inert solvent such as THF. Optionally, the reaction may be carried out in the presence of an organic base such as triethylamine or DIPEA.

m8) Compounds of general formula (IX) above wherein R₃, B, R₁₄, R₁₅, R₅, R^{c} and R^{d} are defined as in formula (I), R₁ is R₆OC(O), R₄ is CN and X is a nitrogen, (—CH₂—NH—) or a single bond connected to a nitrogen which is a member of the B ring may be prepared by reacting a compound of formula (XXXXII) with a compound of general formula (VI) as defined above.

The reaction is generally carried out in an inert solvent such as DMA. Optionally, the reaction may be carried out in the presence of an organic base such as triethylamine or DIPEA.

m9) Compounds of general formula (XXXXII) above may be prepared by essentially the same procedure described in steps m4)-m5) above from a compound of formula (XIV).

n1) Compounds of the general formula (XII) above in which R₁ is R₆OC(O)R₄, is CN and R₃, B, R₅, R₆, R₁₄, R₁₅, X, R^{c} and R^{d} are as defined in formula (I) above may be prepared by reacting a compound of formula (XXXXIII)

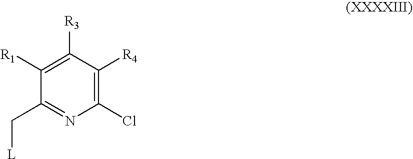

wherein R₁ is R₆OC(O)R₄ is CN, R₃ is as defined in formula (I) and L is a leaving group such as Cl, with a compound of formula (VIII) defined as above.

The reaction may be carried out in an inert solvent such as DMA or EtOH. Optionally, the reaction may be carried out in the presence of an organic base such as triethylamine or DIPEA.

The reaction is generally carried out at elevated temperatures using standard equipment or in a single-node microwave oven.

For some compounds, it is advantageous to carry out the reaction in ethanol in the presence of an organic base such as triethylamine.

n2) Compounds of general formula (XXXXIII) as defined above may be prepared by reacting a compound of formula (XXXXIV), wherein

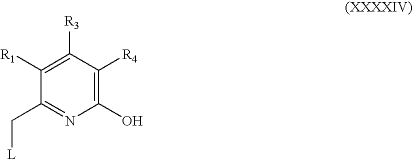

R₁ is R₆OC(O)R₄ is CN, R₃ is as defined in formula (I) and L is a leaving group such as for example Cl, with a chlorinating reagent such as oxalyl chloride, thionyl chloride or POCl₃. Advantageously dimethylformamide may be used. The reaction may be performed in an inert solvent such as DCM.

The reaction is generally carried out at elevated temperatures.

n3) Compounds of the general formula (XXXXIV) as defined above may be prepared by reacting a compound of general formula (XXXXV), wherein R₆ is as defined in formula (I),

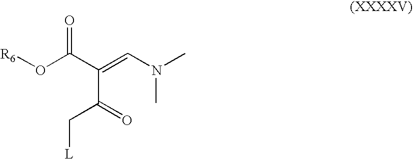

with NC—CH₂C(O)NH₂.

The reaction is generally performed in an inert solvent such as ethanol, optionally in the presence of a strong base such as sodium ethoxide.

o1) Compounds of general formula (II), wherein R₃, B, R₁₄, R₁₅, R₅, R^{c} and R^{d} are defined as in formula (I), R₁ is R₆OC(O), R₄ is CN, R₂ is a substituted (C₁-C₁₂)alkoxy group and X is a single bond or a carbon atom may be prepared by reacting a compound of formula (XXXX) as defined above, with a compound of formula (X)

L-R_{2′} (X)

in which R2′ is a substituted (C₁-C₁₂)alkyl defined as in formula (I) above and L is a leaving group such as chloro, bromo, iodo, triflate (OTf) or tosylate (OTs).

The reaction may be carried out in an inert organic solvent such as DMA, THF or CH₃CN. The reaction may be carried out using standard conditions or in the presence of a suitable base such as sodium hydride, DIPEA or silver carbonate or potassium carbonate. Preferentially silvercarbonate is used.

The reaction may be carried out at ambient temperature or at elevated temperatures using standard equipment or a single node microwave oven.

o2) Compounds of general formula (IV), wherein R₃, B, R₁₄, R₁₅, R₅, R^{c} and R^{d} are defined as in formula (I), R₁ is R₆OC(O), R₄ is CN, R₂ is a substituted (C₁-C₁₂)alkoxy group and X is a nitrogen atom, (—CH₂—NH—) or a single bond connected to a nitrogen atom which is a member of the B-ring may be prepared by reacting a compound of formula (XXXXII) as defined above, with a compound of formula (X)

L-R_{2′} (X)

in which R2′ is a substituted (C₁-C₁₂)alkyl defined as in formula (I) above and L is a leaving group such as chloro, bromo, iodo, triflate (OTf) or tosylate (OTs).

The reaction may be carried out in an inert organic solvent such as DMA, THF or CH₃CN. The reaction may be carried out using standard conditions or in the presence of a suitable base such as sodium hydride, DIPEA or silver carbonate or potassium carbonate. Preferentially silvercarbonate is used.

The reaction may be carried out at ambient temperature or at elevated temperatures using standard equipment or a single node microwave oven.

p) Compounds of general formula (XII) as defined above may be prepared by reacting a compound of formula (IX) with a halogenating reagent, such as thionylchloride, POCl₃ or oxalyl chloride. Optionally the reaction is performed in the presence of DMF.

The reaction may also be carried out in an inert solvent, such as DCM, using trifluoromethanesulfonic anhydride, optionally in the presence of an organic base such as TEA or DIPEA at or below r.t.

q) The preparation of compounds of the general formula (XXXXVI), in which B, R₁₄ and R₁₅ are defined as for formula (I) with the exception that R₁₄ is connected to the same atom as X, and X is defined as a single bond, comprises the below step;

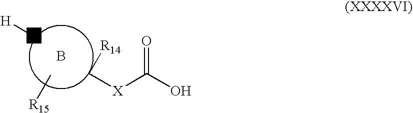

q1) Reacting the corresponding (XXXXVII) with R₁₄-L, wherein L is a suitable leaving group, such as chloro, bromo, iodo,

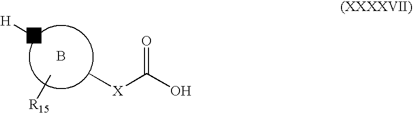

triflate (OTf), mesylate (OMs) or tosylate (OTs) to form compounds of the general formula (XXXXVI), using standard conditions or in the presence of a mixture of BuLi and diisopropylamine (to form LDA).

The preparation of compounds of the formula (III) comprises the below processes. (r1-r3)

r1) A compound of the formula LR^{c}R^{d} wherein L is a suitable leaving group, such as chloro, bromo, iodo could be transformed to the corresponding compound (III) using a sequence of reactions using first SMOPS* (*Baskin and Wang. Tetrahedron Letters, 2002, 43, 8479-83. See esp. page 8480, left hand column.) followed by hydrolysis using a base like NaOMe in an inert solvent like DMSO at room temperature. Followed by treatment by NH₂OSO₃H and NaOAc to give a compound of formula (III).

r2) A compound of the formula LSO₂R^{c}R^{d} wherein L is a suitable leaving group, such as chloro, bromo, iodo could be reacted with ammonium hydroxide or H₂NR₅ in an inert solvent such as DCM to give a compound of formula (III).

r3) A compound of the formula LR^{c}R^{d} wherein L is a suitable leaving group, such as chloro, bromo, iodo could be transformed to the corresponding compound (III) using a sequence of reactions first Na₂SO₃, followed by a using a reagent such as PCl₅, POCl₃ or SOCl₂, followed by ammonium hydroxide or H₂NR₅ to give a compound of formula (III).

At any stage in the synthesis of amine substituted pyridines, a halogen substituent in the 2, 4 or 6 position of the pyridine can be substituted with azide using known techniques. The azide can be reduced to the corresponding amine. These amines can subsequently be alkylated or acylated using known methods or with an alkylhalide or acylhalide, respectively.

Persons skilled in the art will appreciate that an acid can be transformed to the corresponding activated ester such as an acid chloride, followed by reaction with a thiol, R₁₆SH to give thioesters, R₁₆SC(O).

Persons skilled in the art will appreciate that an acid can be transformed to the corresponding activated ester such as an acid chloride, followed by reaction with a alcohol, R₆OH to give esters, R₆OC(O).

Persons skilled in the art will appreciate that a compound of formula (III) could be alkylated at the carbon atom in the alpha position to the sulfonamide using an alkylhalide. Preferably under basic conditions using a strong base such as sodium hydride.

Persons skilled in the art will appreciate that a nitrogen substituent at the 3 position of a pyridine could be replaced by a thioether chain, R₁₇S—, using known techniques or R₁₇SSR₁₇ and tert-Butylnitrite.

Persons skilled in the art will appreciate that a thioketone could be made from the corresponding ketone using known techniques or using Lawessons reagent.

Persons skilled in the art will appreciate that a pyridine N-oxide could be formed by from a pyridine using an oxidizing agent such as Urea hydrogen peroxide or hydrogen peroxide, with or without the presence of trifluoroaceticanhydrid.

The compounds of the invention may be isolated from their reaction mixtures using conventional techniques.

It will be appreciated that by those skilled in the art that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldiphenylsilyl or trimethylsilyl) and tetrahydropyranyl. Suitable protecting groups for carboxylic acids include (C₁-C₆)alkyl or benzyl esters. Suitable protecting groups for amino include allyl, t-butyloxycarbonyl, benzyloxycarbonyl, 2-(trimethylsilyl)ethoxymethyl or 2-trimethylsilylethoxycarbonyl (Teoc).

The protection and deprotection of functional groups may take place before or after any reaction in the above mentioned processes.

Persons skilled in the art will appreciate that, in order to obtain compounds of the invention in an alternative, and on some occasions, more convenient, manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

Persons skilled in the art will appreciate that starting materials for any of the above processes can in some cases be commercially available.

Persons skilled in the art will appreciate that processes could for some starting materials above be found in the general common knowledge.

The type of chemistry involved will dictate the need for protecting groups as well as sequence for accomplishing the synthesis.

The use of protecting groups is fully described in “Protective groups in Organic Chemistry”, edited by J W F McOmie, Plenum Press (1973), and “Protective Groups in Organic Synthesis”, 3^{rd} edition, T. W. Greene & P. G. M Wutz, Wiley-Interscience (1999).

Protected derivatives of the invention may be converted chemically to compounds of the invention using standard deprotection techniques (e.g. under alkaline or acidic conditions). The skilled person will also appreciate that certain compounds of Formula (II)-(XXXXVII) and (LI)-(LV) may also be referred to as being “protected derivatives”

Compounds of the invention may also contain one or more asymmetric carbon atoms and may therefore exhibit optical and/or diastereoisomerism. Diastereoisomers may be separated using conventional techniques, e.g. chromatography or crystallization. The various stereisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerization, or by derivatisation, for example with a homochiral acid followed by separation of the diasteromeric derivatives by conventional means (e.g. HPLC, chromatography over silica or crystallization). Stereo centers may also be introduced by asymmetric synthesis, (e.g. metalloorganic reactions using chiral ligands). All stereoisomers are included within the scope of the invention. It will also be understood that some of the compounds described in the processes above may exhibit the phenomenon of tautomerism and the processes described above includes any tautomeric form.

All novel intermediates form a further aspect of the invention.

Salts of the compounds of formula (I) may be formed by reacting the free acid, or a salt thereof, or the free base, or a salt or a derivative thereof, with one or more equivalents of the appropriate base (for example ammonium hydroxide optionally substituted by C₁-C₆-alkyl or an alkali metal or alkaline earth metal hydroxide) or acid (for example a hydrohalic (especially HCl), sulphuric, oxalic or phosphoric acid). The reaction may be carried out in a solvent or medium in which the salt is insoluble or in a solvent in which the salt is soluble, e.g. water, ethanol, tetrahydrofuran or diethyl ether, which may be removed in vacuo, or by freeze drying. The reaction may also carried out on an ion exchange resin. The non-toxic physiologically acceptable salts are preferred, although other salts may be useful, e.g. in isolating or purifying the product.

### Pharmacological Data

Functional inhibition of- the P2Y₁₂ receptor can be measured by in vitro assays using cell membranes from P2Y₁₂ transfected CHO-cells, the methodology is indicated below.

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation

y=A+((B−A)/(1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.

Most of the compounds of the invention have an activity, when tested in the functional inhibition of 2-Me-S-ADPinduced P2Y₁₂ signalling assay described, at a concentration of around 2 μM or below.

For example the compounds described in Examples 4 and 12 gave the following test result in the functional inhibition of 2-Me-S-ADPinduced P2Y₁₂ signalling assay described.

| | IC₅₀(μM) |
|---|---|
| Example 4 | 0.28 |
| Example 12 | 0.13 |

The compounds of the invention act as P2Y₁₂ receptor antagonists and are therefore useful in therapy. Thus, according to a further aspect of the invention there is provided a compound of formula (I), or a pharmaceutically acceptable salt thereof, for use in therapy.

Thus, according to another further aspect of the invention there is provided a compound of formula (I), or a pharmaceutically acceptable salt thereof, for use as a medicament.

In a further aspect there is provided the use of a compound of formula (I), or a pharmaceutically acceptable salt thereof, for the manufacture of a medicament for treatment of a platelet aggregation disorder. In another aspect of the invention there is provided the use of a compound of formula (I), or a pharmaceutically acceptable salt thereof, for the manufacture of a medicament for the inhibition of the P2Y₁₂ receptor.

In yet another aspect of the invention there is provided a compound of formula (I), or a pharmaceutically acceptable salt thereof, for use as an inhibitor of the P2Y₁₂ receptor.

In still another aspect of the invention there is provided a compound of formula (I), or a pharmaceutically acceptable salt thereof, for use in the treatment of platelet aggregation disorder.

The compounds are useful in therapy, especially adjunctive therapy, particularly they are indicated for use as: inhibitors of platelet activation, aggregation and degranulation, promoters of platelet disaggregation, anti-thrombotic agents or in the treatment or prophylaxis of unstable angina, coronary angioplasty (PTCA), myocardial infarction, perithrombolysis, primary arterial thrombotic complications of atherosclerosis such as thrombotic or embolic stroke, transient ischaemic attacks, peripheral vascular disease, myocardial infarction with or without thrombolysis, arterial complications due to interventions in atherosclerotic disease such as angioplasty, endarterectomy, stent placement, coronary and other vascular graft surgery, thrombotic complications of surgical or mechanical damage such as tissue salvage following accidental or surgical trauma, reconstructive surgery including skin and muscle flaps, conditions with a diffuse thrombotic/platelet consumption component such as disseminated intravascular coagulation, thrombotic thrombocytopaenic purpura, haemolytic uraemic syndrome, thrombotic complications of septicaemia, adult respiratory distress syndrome, anti-phospholipid syndrome, heparin-induced thrombocytopaenia and pre-eclampsia/eclampsia, or venous thrombosis such as deep vein thrombosis, venoocclusive disease, haematological conditions such as myeloproliferative disease, including thrombocythaemia, sickle cell disease; or in the prevention of mechanically-induced platelet activation in vivo, such as cardio-pulmonary bypass and extracorporeal membrane oxygenation (prevention of microthromboembolism), mechanically-induced platelet activation in vitro, such as use in the preservation of blood products, e.g. platelet concentrates, or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in other inflammatory conditions such as asthma, in which platelets and platelet-derived factors are implicated in the immunological disease process.

According to the invention there is further provided the use of a compound according to the invention in the manufacture of a medicament for the treatment of the above disorders. In particular the compounds of the invention are useful for treating myocardial infarction, thrombotic stroke, transient ischaemic attacks, peripheral vascular disease and angina, especially unstable angina. The invention also provides a method of treatment of the above disorders which comprises administering to a patient suffering from such a disorder a therapeutically effective amount of a compound according to the invention.

In a further aspect the invention provides a pharmaceutical composition comprising a compound of formula (I), or a pharmaceutically acceptable salt thereof, together with a pharmaceutically acceptable diluent, adjuvant and/or carrier.

The compounds may be administered topically, e.g. to the lung and/or the airways, in the form of solutions, suspensions, HFA aerosols and dry powder formulations; or systemically, e.g. by oral administration in the form of tablets, pills, capsules, syrups, powders or granules, or by parenteral administration in the form of sterile parenteral solutions or suspensions, by subcutaneous administration, or by rectal administration in the form of suppositories or transdermally.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction.

Dry powder formulations and pressurised HFA aerosols of the compounds of the invention may be administered by oral or nasal inhalation. For inhalation the compound is desirably finely divided. The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol or another polyol. Suitable carriers include sugars and starch. Alternatively the finely divided compound may be coated by another substance. The powder mixture may also be dispensed into hard gelatine capsules, each containing the desired dose of the active compound.

Another possibility is to process the finely divided powder into spheres, which break up during the inhalation procedure. This spheronized powder may be filled into the drug reservoir of a multidose inhaler, e.g. that known as the Turbuhaler® in which a dosing unit meters the desired dose which is then inhaled by the patient. With this system the active compound with or without a carrier substance is delivered to the patient.

The pharmaceutical composition comprising the compound of the invention may conveniently be tablets, pills, capsules, syrups, powders or granules for oral administration; sterile parenteral or subcutaneous solutions, suspensions for parenteral administration or suppositories for rectal administration.

For oral administration the active compound may be admixed with an adjuvant or a carrier, e.g. lactose, saccharose, sorbitol, mannitol, starches such as potato starch, corn starch or amylopectin, cellulose derivatives, a binder such as gelatine or polyvinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved either in a readily volatile organic solvent or an aqueous solvent.

For the preparation of soft gelatine capsules, the compound may be admixed with e.g. a vegetable oil or polyethylene glycol. Hard gelatine capsules may contain granules of the compound using either the above mentioned excipients for tablets, e.g. lactose, saccharose, sorbitol, mannitol, starches, cellulose derivatives or gelatine. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and carboxymethylcellulose as a thickening agent or other excipients known to those skilled in art.

The invention will be further illustrated with the following non-limiting examples:

### EXAMPLES

### General Experimental Procedure

Mass spectra was recorded on a Finnigan LCQ Duo ion trap mass spectrometer equipped with an electrospray interface (LC-MS) or LC-MS system consisting of a Waters ZQ using a LC-Agilent 1100 LC system. ¹H NMR measurements were performed on a Varian Mercury VX 400 spectrometer, operating at a 1H frequency of 400 and Varian UNITY plus 400, 500 and 600 spectrometers, operating at 1H frequencies of 400, 500 and 600 respectively. Chemical shifts are given in ppm with the solvent as internal standard. Protones on heteroatoms such as NH and OH protons are only reported when detected in NMR and can therfore be missing.

HPLC separations were performed on a Waters YMC-ODS AQS-3 120 Angstrom 3×500 mm or on a Waters Delta Prep Systems using Kromasil C8, 10 μm columns.

Straight phase chromatography was performed using Biotage silica gel 40S, 40M, 12i or Merck silica gel 60 (0.063-0.200 mm). Flash-chromatography was performed using either standard glass- or plastic-columns or on a Biotage Horizon system

Purification Method A: The purification system and LC-MS system used in purification Method A, referred to in some of the Examples below, was Waters Fraction Lynx I Purification System Column: Sunfire Prep C₁₈, 5 μm OBD, 19×150 mm column. Gradient 5-95% CH₃CN in 0.1 mM HCOOH (pH=3). MS triggered fraction collection was used. Mass spectra were recorded on either Micromass ZQ single quadropole or a Micromass quattro micro, both equipped with a pneumatically assisted electrospray interface.

Reactions performed in a microwave reactor were performed in a Personal Chemistry Smith Creator, Smith synthesizer or an Emrys Optimizer.

IUPAC names were generated with ACDLabs Name: Release 9:00, Product version 9.04.

The GTPγS values (IC₅₀ in μM) mentioned in the examples below were measured by the method described below:

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation

y=A+((B−A)/(1+((C/x)̂D)))

and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.

### List of Used Abbreviations:

| Abbreviation | Explanation |
|---|---|
| AcOH | acetic acid |
| aq | Aqueous |
| Boc tert- | butyloxycarbonyl |
| br | Broad |
| BSA | Bovine Serum Albumine |
| CDI | Carbonyldiimidazole |
| d | Doublet |
| dba | 1,5-diphenylpenta-1,4-dien-3-one |
| DBU | 1,8-diazabicyclo[5.4.0]undec-7-ene |
| DCM | Dichloromethane |
| DDQ | 2,3-Dichloro-5,6-dicyano-1,4-benzoquinone |
| DIPEA | N,N-Diisopropylethylamine |
| DMA | N,N-Dimethylacetamide |
| DMAP | N,N-dimethylaminopyridine |
| DMF | N,N-dimethylformamide |
| DMSO | Dimethylsulphoxide |
| EDCI | N-[3-(dimethylamino)propyl]-N′-ethylcarbodiimide |
| | hydrochloride |
| EtOAc | Ethyl acetate |
| EtOH | Ethanol |
| FA | formic acid |
| g | gram |
| h | hours |
| HEPES | [4-(2-hydroxyethyl)-1-piperazineethanesulfonic |
| | acid |
| HFA | Hydrofluoroalkanes |
| HOAc | Acetic acid |
| HOBt | 1-Hydroxybenzotriazole |
| HPLC | High-performance liquid chromatography |
| Hz | Hertz |
| IPA | isopropylalcohol |
| iPr | isopropyl |
| J | Coupling constant |
| LC | Liquid chromatography |
| m | Multiplet |
| MeCN | acetonitrile |
| MeOH | Methanol |
| mg | milligram |
| MHz | Megahertz |
| min | minutes |
| mL | Millilitre |
| MS | Mass spectra |
| Ms | methylsulfonyl |
| MTBE | methyl tert-butylether |
| NBS | N-bromsuccinimide |
| NCS | N-chlorosuccinimide |
| NMP | N-metylpyrrolidone |
| NMR | Nuclear magnetic resonance |
| OAc | acetate |
| Ph | Phenyl |
| q | Quartet |
| r.t | room temperature |
| s | singlet |
| t | triplet |
| TB | Tyrodes Buffer |
| TBTU | N-[(1H-1,2,3-benzotriazol-1- |
| | yloxy)(dimethylamino)methylene]-N- |
| | methylmethanaminium tetrafluoroborate |
| TEA | Triethylamine |
| Tf | trifluoromethylsulfonyl |
| TFA | Trifluoroacetic acid |
| THF | Tetrahydrofurane |
| TMEDA | N,N,N′,N′-tetramethylethylendiamine |
| Ts | p-toluenesulfonyl |

### Sulfone Amides

### Synthesis of Sulfone Amides

The synthesis of the sulfonamides used in the examples below was made with one of the three methods described below:

i) By reacting the corresponding sulfonyl chloride with ammonia in THF or MeOH or by treatment with ammonium hydroxide in methylene chloride. The sulfonamides obtained was used without further purification.
ii) By essentially following the procedure described by Seto, T. et. al. in J. Organic Chemistry, Vol 68, No 10 (2003), pp. 4123-4125.
or
iii) By essentially following the procedure described by Wang, Z et. al. in Tetrahedron Letters, Vol 43 (2002), pp 8479-8483.

### Synthesis of Sulfamides.

The different sulfamides in the examples below (like N-Methyl-N-phenylsulfamide) were prepared by essentially the same procedure as described in Example 13(c) by replacing N-methylaniline with the appropriate amine.

N-Methyl-N-phenylsulfamide (Typical Procedure Taken from Ex. 13(c)

Chlorosulfonyl isocyanate (3.7 mL, 42.4 mmol) was dissolved in dry DCM (40 mL), the solution was cooled to 0° C. and tert-butanol (3.98 mL, 42.4 mmol) was added drop wise. The reaction mixture was stirred at r.t for 2 h, the solution was cooled to 0° C. and N-methylaniline (4.61 mL, 42.4 mmol) and TEA (8.85 mL, 63.6 mmol) dissolved in dry DCM (20 mL) were added drop wise through a dropping funnel. The reaction was stirred at r.t for 3 h, water was added and the organic phase was separated and dried (phase separator, Isolute) and concentrated in vacuo. The residue was dissolved in DCM (40 mL) and trifluoroacetic acid (32.7 mL, 423 mmol) was added. The reaction was stirred at r.t for 20 min, the solvent was concentrated in vacuo and co evaporated with DCM (3×). The crude product was purified with flash column chromatography, using a mixture of heptane:EtOAc 70:30 as eluent, to give N-methyl-N-phenylsulfamide. Yield: 5.96 g (76%).

¹H-NMR (500 MHz, CDCl₃) δ 3.22 (3H, s), 4.77 (2H, s), 7.28-7.33 (1H, m), 7.36-7.42 (4H, m).

MSm/z: 187 (M+1).

### Example 1

### 1-{5-Acetyl-3-cyano-6-[(2-oxopyrrolidin-1-yl)methyl]pyridin-2-yl}-N-(benzylsulfonyl)piperidine-4-carboxamide

### (a) Ethyl 3-oxo-4-(2-oxopyrrolidin-1-yl)butanoate

2-Pyrrolidone (27 g, 0.3 mol) in toluene (100 mL) was added drop wise to a solution of NaH (15 g, 0.64 mol) in 2-methyl tetrahydrofurane (250 mL) at −5° C., the reaction mixture was stirred at −5° C. for 2 h. Ethyl 4-chloroacetoacetate (50 g, 0.3 mol) dissolved in toluene (100 mL) was added drop wise to the solution at −5° C., the reaction mixture was stirred at r.t over night. Acetic acid (36.5 mL, 0.64 mmol) in water (250 mL) was added, the organic solvent was separated, dried (MgSO₄) and concentrated in vacuo to give ethyl 3-oxo-4-(2-oxopyrrolidin-1-yl)butanoate. The crude product was used in the next step without further purification. Yield: 57.5 g (89%).

¹H-NMR (500 MHz, DMSO-d₆) δ 1.19 (3H, t), 1.96 (2H, pentet), 2.25 (2H, t), 3.31 (2H, d), 3.64 (2H, s), 4.10 (2H, q), 4.20 (2H, s).

### (b) Ethyl 5-cyano-6-hydroxy-2-[(2-oxopyrrolidin-1-yl)methyl]nicotinate

Ethyl 3-oxo-4-(2-oxopyrrolidin-1-yl)butanoate (4.1 g, 19.3 mmol) was dissolved in EtOH (80 mL), N,N-dimethylformamide dimethyl acetal (2.7 mL, 20.2 mmol) was added. The reaction mixture was stirred at 50° C. for 1.5 h, the solution was cooled to r.t and DIPEA (0.67 mL, 3.85 mmol) and malononitrile (1.4 mL, 22.23 mmol) in EtOH (20 mL) were added. The reaction was stirred at r.t for 2.5 h, a solution of acetic acid (1.3 mL, 23.1 mmol) in water (4 mL) was added and the EtOH was concentrated in vacuo. Water was added and the precipitate was filtered, the solid material was washed with water and MeOH and dried to give ethyl 5-cyano-6-hydroxy-2-[(2-oxopyrrolidin-1-yl)methyl]nicotinate. Yield: 4.3 g (77%).

¹H-NMR (500 MHz, DMSO-d₆) δ 1.28 (3H, t), 1.96 (2H, quintet), 2.25 (2H, t), 4.23 (2H, q), 4.72 (2H, s), 8.47 (1H, s), 12.61 (1H, s).

Note: one signal (1H) overlapps with the H₂O signal in the DMSO.

MSm/z: 290 (M+1), 288 (M−1).

### (c) Ethyl 6-[4-(tert-butoxycarbonyl)piperidin-1-yl]-5-cyano-2-[(2-oxopyrrolidin-1-yl)methyl]nicotinate

DIPEA (19.7 mL, 113 mmol) and bromo-tris-pyrrolidino-phosphonium hexafluorophospate (13.2 g, 28.3 mmol) were added to a solution of ethyl 5-cyano-6-hydroxy-2-[(2-oxopyrrolidin-1-yl)methyl]nicotinate (5.5 g, 18.9 mmol) in DCM (50 mL), the reaction mixture was stirred for 15 min at rt. 4-tert-Butyl-carboxylicacid-piperidine (4.14 g, 22.3 mmol) in DCM (10 mL) was added, the reaction mixture was stirred at r.t over night. 1M HCl was added and the organic solvent was separated and concentrated in vacuo, the residue was purified by HPLC (Kromasil C8, 250×50 Idmm using a gradient of 30% to 90% MeCN over 30 min with an acidic second eluent (H₂O/MeCN/FA:95:5:0.2) to give ethyl 6-[4-(tert-butoxycarbonyl)piperidin-1-yl]-5-cyano-2-[(2-oxopyrrolidin-1-yl)methyl]nicotinate. Yield: 6.3 g (74%).

¹H-NMR (600 MHz, CDCl₃) δ 1.35 (3H, t), 1.43 (9H, s), 1.72-1.80 (2H, m), 1.94-2.00 (2H, m), 2.05-2.11 (2H, m), 2.42-2.46 (2H, m), 2.49-2.54 (1H, m), 3.28-3.34 (2H, m), 3.44-3.49 (2H, m), 4.30 (2H, q), 4.42-4.47 (2H, m), 4.89 (2H, s), 8.35 (1H, s).

MSm/z: 457 (M+1), 455 (M−1).

### (d) 6-[4-(tert-Butoxycarbonyl)piperidin-1-yl]-5-cyano-2-[(2-oxopyrrolidin-1-yl)methyl]nicotinic acid

Ethyl 6-[4-(tert-butoxycarbonyl)piperidin-1-yl]-5-cyano-2-[(2-oxopyrrolidin-1-yl)methyl]nicotinate (6.3 g, 13.8 mmol) was dissolved in EtOH (40 mL) and MeCN (20 mL). NaOH (10 mL, 4.97 M) was added, the reaction mixture was heated in the microwave at 80° C. for 5 min. Formic acid and water were added to pH 3, the solution was cooled in the fridge for 1 h. The precipitate was filtered off and washed with acidic water and 2-propanol. The solid material was dried under vacuum and co-evaporated from MeCN (×4) to give 6-[4-(tert-butoxycarbonyl)piperidin-1-yl]-5-cyano-2-[(2-oxopyrrolidin-1-yl)methyl]nicotinic acid. Yield: 5.64 g (95.4%)

¹H-NMR (600 MHz, CDCl₃) δ 1.44 (9H, s), 1.73-1.81 (2H, m), 1.95-2.02 (2H, m), 2.07-2.13 (2H, m), 2.45-2.49 (2H, m), 2.50-2.56 (1H, m), 3.30-3.36 (2H, m), 3.53-3.57 (2H, m), 4.43-4.49 (2H, m), 4.86 (2H, s), 7.99 (1H, s), 8.34 (1H, s).

MSm/z: 429 (M+1), 427 (M−1).

### (e) tert-Butyl 1-{5-(chlorocarbonyl)-3-cyano-6-[(2-oxopyrrolidin-1-yl)methyl]pyridin-2-yl}piperidine-4-carboxylate

6-[4-(tert-Butoxycarbonyl)piperidin-1-yl]-5-cyano-2-[(2-oxopyrrolidin-1-yl)methyl]nicotinic acid (4.2 g, 9.80 mmol) was dissolved in DCM (100 mL) and cooled to 0° C. Oxalyl chloride (1.83 mL, 21.6 mmol) and 10 drops of DMF were added, the reaction was stirred at r.t for 1 h. The solvent was concentrated in vacuo and used in the next step without further purification.

### (f) tert-Butyl 1-{3-fluoro-5-(fluorocarbonyl)-6-[(2-oxopyrrolidin-1-yl)methyl]pyridin-2-yl}piperidine-4-carboxylate

Pyridine (0.126 mL, 1.3 mmol) was added to a solution of 6-[4-(tert-Butoxycarbonyl)piperidin-1-yl]-5-cyano-2-[(2-oxopyrrolidin-1-yl)methyl]nicotinic acid (0.506 g, 1.2 mmol in dry DCM (6 mL) under nitrogen. The reaction mixture was stirred 5 minutes at room temperature. Cyanuric fluoride (1.2 mmol, 0.162 g) was added. The reaction mixture was stirred at room temperature under nitrogen for 16 h.

Water (20 mL) was added. Extra pyridine (0.2 mL) was added. The aqueous phase was extracted with DCM (3×20 mL). Combined organic phases were dried with sodium sulphate and concentrated. This gave 0.543 g.

¹H NMR (CDCl₃, 400 MHz) δ 1.30 (9H, s), 1.71-1.83 (2H, m), 1.92-2.02 (2H, m), 2.15 (2H, q, J=7.5 Hz), 2.49 (2H, t, J=8.1 Hz), 2.45-2.62 (1H, m), 3.18-3.31 (2H, m), 3.51 (2H, t, J=7.0 Hz), 4.32-4.42 (2H, m), 4.82 (2H, s), 7.66 (1H, d, J=14.2 Hz).

### (g) tert-Butyl 1-{5-acetyl-3-cyano-6-[(2-oxopyrrolidin-1-yl)methyl]pyridin-2-yl}piperidine-4-carboxylate

tert-Butyl 1-{5-(chlorocarbonyl)-3-cyano-6-[(2-oxopyrrolidin-1-yl)methyl]pyridin-2-yl}piperidine-4-carboxylate (1.04 g, 2.33 mmol) was dissolved in dry THF (25 mL), ferric acetylacetonate (247 mg, 0.70 mmol) and methyl magnesium chloride (2.34 mL, 7 mmol, 3 M in THF) was added dropwise. The reaction was stirred at r.t for 30 min, the reaction was quenched with MeOH (5 mL) and partitioned between water and EtOAc. The aqueous phase was extracted with EtOAc (×2), the combined organic phase was dried (Na₂SO₄), concentrated in vacuo and purified by HPLC (Kromasil C8, 250×50 Idmm using a gradient of 30% to 80% MeCN over 45 min with an acidic second eluent (H₂O/MeCN/FA:95:5:0.2)) to give tert-butyl 1-{5-acetyl-3-cyano-6-[(2-oxopyrrolidin-1-yl)methyl]pyridin-2-yl}piperidine-4-carboxylate. Yield: 190 mg (19%).

¹H-NMR (600 MHz, CDCl₃) δ 1.45 (9H, s), 1.75-1.82 (2H, m), 1.97-2.03 (2H, m), 2.06-2.13 (2H, m), 2.44-2.48 (2H, m), 2.50 (3H, s), 2.52-2.58 (1H, m), 3.33-3.39 (2H, m), 3.47-3.50 (2H, m), 4.47-4.53 (2H, m), 4.86 (2H, s), 8.17 (1H, s).

MSm/z: 427 (M+1), 425 (M−1).

The coupling reaction for some of the compounds in the Examples below (that are referring to this procedure) was performed at 0° C. instead of r.t. and by using the acid fluoride described in step (f) above. This is indicated in each specific. (For description of similar chemistry see also Fürstner, A. et al, J. Org. Chem. 2004, pp. 3943-3949.)

### (h) 1-{5-Acetyl-3-cyano-6-[(2-oxopyrrolidin-1-yl)methyl]pyridin-2-yl}piperidine-4-carboxylic acid

tert-Butyl 1-{5-acetyl-3-cyano-6-[(2-oxopyrrolidin-1-yl)methyl]pyridin-2-yl}piperidine-4-carboxylate (190 mg, 0.45 mmol) was dissolved in DCM (2 mL), TFA (2 mL, 26 mmol) was added. The reaction mixture was stirred at r.t for 1 h, the solvent was concentrated in vacuo and the crude product was coevaporated with DCM (3×). The crude product was used in the next step without purification.

MSm/z: 371 (M+1), 369 (M−1)

### (i) 1-{5-Acetyl-3-cyano-6-[(2-oxopyrrolidin-1-yl)methyl]pyridin-2-yl}-N-(benzylsulfonyl)piperidine-4-carboxamide

1-{5-Acetyl-3-cyano-6-[(2-oxopyrrolidin-1-yl)methyl]pyridin-2-yl}piperidine-4-carboxylic acid (36 mg, 0.097 mmol) was dissolved in DCM (4 mL) and added to 1-phenylmethanesulfonamide (18.7 mg, 0.11 mmol). Bromo-tris-pyrrolidino-phosphonium hexafluorophosphate (68 mg, 0.15 mmol) and DIPEA (0.1 mL, 58 mmol) were added. The reaction mixture was stirred at r.t over night, 2% KHSO₄ was added and the aqueous phase was extracted with DCM and the organic phase was dried (phase separator) and concentrated in vacuo. The residue was purified by preparative reverse phase HPLC (Kromasil C8, 10 μm, using an increasing gradient of MeCN with an acidic second eluent (H₂O/MeCN/FA 95:5:0.2)) to give 1-{5-acetyl-3-cyano-6-[(2-oxopyrrolidin-1-yl)methyl]pyridin-2-yl}-N-(benzylsulfonyl)piperidine-4-carboxamide. Yield: 35.3 mg (53%).

¹H-NMR (600 MHz, CDCl₃) δ 1.73-1.89 (6H, m), 2.05-2.10 (2H, m), 2.38 (2H, t), 2.50 (3H, s), 3.20-3.24 (1H, m), 3.45 (2H, t), 4.51-4.56 (2H, m), 4.62 (2H, s), 4.83 (2H, s), 7.32-7.42 (5H, m), 8.19 (1H, s), 10.06 (1H, s).

MSm/z: 524 (M+1), 522 (M−1).

GTPγS(IC₅₀ μM): 0.283

### Example 2

### N-(Benzylsulfonyl)-1-{5-butyryl-3-cyano-6-[(2-oxopyrrolidin-1-yl)methyl]pyridin-2-yl}piperidine-4-carboxamide

### (a) Ethyl 6-[4-(tert-butoxycarbonyl)piperidin-1-yl]-5-cyano-2-[(2-oxopyrrolidin-1-yl)methyl]nicotinate

Prepared according to Example 1(g) from tert-butyl 1-{5-(florocarbonyl)-3-cyano-6-[(2-oxopyrrolidin-1-yl)methyl]pyridin-2-yl}piperidine-4-carboxylate

(Example 1(f)) (4.35 g, 10.1 mmol) by using propyl magnesium bromide in place of methylmagnesium chloride to give ethyl 6-[4-(tert-butoxycarbonyl)piperidin-1-yl]-5-cyano-2-[(2-oxopyrrolidin-1-yl)methyl]nicotinate. This reaction was performed at 0° C. Yield: 1.97 g (43%).

MSm/z: 455 (M+1), 453 (M−1).

### (b) 1-{5-Butyryl-3-cyano-6-[(2-oxopyrrolidin-1-yl)methyl]pyridin-2-yl}piperidine-4-carboxylic acid

Prepared according to Example 1(h) from ethyl 6-[4-(tert-butoxycarbonyl)piperidin-1-yl]-5-cyano-2-[(2-oxopyrrolidin-1-yl)methyl]nicotinate (445 mg, 0.98 mmol) to give 1-{5-butyryl-3-cyano-6-[(2-oxopyrrolidin-1-yl)methyl]pyridin-2-yl}piperidine-4-carboxylic acid. Yield: 390 mg (100%).

MSm/z: 399 (M+1), 397 (M−1)

### (c) N-(Benzylsulfonyl)-1-{5-butyryl-3-cyano-6-[(2-oxopyrrolidin-1-yl)methyl]pyridin-2-yl}piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (96 mg, 0.24 mmol) to give N-(benzylsulfonyl)-1-{5-butyryl-3-cyano-6-[(2-oxopyrrolidin-1-yl)methyl]pyridin-2-yl}piperidine-4-carboxamide. Yield: 83 mg (62%).

¹H-NMR (500 MHz, DMSO-d₆) δ 0.92 (3H, t), 1.55-1.64 (4H, m), 1.80-1.85 (2H, m), 2.00 (2H, quintet), 2.29 (2H, t), 2.58-2.64 (1H, m), 2.93 (2H, t), 3.17-3.23 (2H, m), 3.43 (2H, t), 4.48-4.52 (2H, m), 4.66-4.70 (4H, m), 7.28-7.43 (5H, m), 8.64 (1H, s), 11.59 (1H, s).

MSm/z: 552 (M+1), 550 (M−1).

GTPγS(IC₅₀ μM): 0.022

### Example 3

### 1-{5-Acetyl-3-cyano-6-[(2-oxopiperidin-1-yl)methyl]pyridin-2-yl}-N-(benzylsulfonyl)piperidine-4-carboxamide

### (a) Ethyl 3-oxo-4-(2-oxopiperidin-1-yl)butanoate

Delta-valerolaktam (3.16 g, 31.9 mmol) in toluene (12.5 mL) was added drop wise to a solution of NaH (1.53 g, 63.8 mmol) in 2-methyl tetrahydrofurane at −5° C., the reaction mixture was stirred at −5° C. for 2 h. Ethyl 4-chloroacetoacetate (5 g, 30.4 mmol) dissolved in toluene (12.5 mL) was added drop wise to the solution at −5° C., the reaction mixture was stirred at r.t over night. Acetic acid (3.5 mL, 60.8 mmol) in water (25 mL) was added, the organic solvent was separated, dried (MgSO₄) and concentrated in vacuo to give ethyl 3-oxo-4-(2-oxopiperidin-1-yl)butanoate. Yield: 6.3 g (91%).

¹H-NMR (500 MHz, CDCl₃) δ 1.26 (3H, t), 1.84 (4H, m), 2.42 (2H, m), 3.30 (2H, m), 3.64 (2H, s), 4.18 (2H, q), 4.26 (2H, s).

MSm/z: 228

### (b) Ethyl 5-cyano-6-oxo-2-[(2-oxopiperidin-1-yl)methyl]-5,6-dihydropyridine-3-carboxylate

Ethyl 3-oxo-4-(2-oxopiperidin-1-yl)butanoate (6.3 g, 27.7 mmol) was dissolved in EtOH (100 mL), N,N-dimethylformamide dimethyl acetal (3.9 mL, 29.1 mmol) was added. The reaction mixture was stirred at 50° C. for 1.5 h, the solution was cooled to r.t and DIPEA (0.97 mL, 5.54 mmol) and malononitrile (2 mL, 31.9 mmol) in EtOH (25 mL) were added. The reaction was stirred at r.t for 2.5 h, a solution of acetic acid (1.9 mL, 32.3 mmol) in water (10 mL) was added and the EtOH was concentrated in vacuo. Water was added and the precipitate was filtered, the solid material was washed with water and MeOH and dried to give ethyl 5-cyano-6-oxo-2-[(2-oxopiperidin-1-yl)methyl]-5,6-dihydropyridine-3-carboxylate. Yield: 3.43 g (41%).

¹H-NMR (500 MHz, DMSO-d₆) δ 1.29 (3H, t), 1.76 (4H, m), 2.28 (2H, m), 3.31 (2H, m), 4.24 (2H, q), 4.81 (2H, s), 8.47 (1H, s).

MSm/z: 304 (M+1)

### (c) Ethyl 6-[4-(tert-butoxycarbonyl)piperidin-1-yl]-5-cyano-2-[(2-oxopiperidin-1-yl)methyl]nicotinate

DIPEA (1.3 mL, 7.47 mmol), bromo-tris-pyrrolidino-phosphonium hexafluorophospate (1.5 g, 3.3 mmol) and 4-piperidinecarboxylic acid tert-butyl ester (0.6 g, 3.24 mmol) were added to a solution of ethyl 5-cyano-6-oxo-2-[(2-oxopiperidin-1-yl)methyl]-5,6-dihydropyridine-3-carboxylate (0.9 g, 2.07 mmol) in DCM (25 mL), the reaction was stirred for 30 min at rt. Sat. NaHCO₃ was added and the organic solvent was concentrated in vacuo, the residue was purified by HPLC (Kromasil C8 using an increasing gradient of MeCN in HOAc(aq, 0.2%). The organic solvent was concentrated in vacuo, water and EtOAc was added and the organic solvent was separated, dried (MgSO₄) and concentrated in vacuo to give ethyl 6-[4-(tert-butoxycarbonyl)piperidin-1-yl]-5-cyano-2-[(2-oxopiperidin-1-yl)methyl]nicotinate. Yield: 1.11 g (80%).

¹H-NMR (500 MHz, CDCl₃) δ 1.37 (3H, t), 1.45 (9H, s), 1.73-1.89 (6H, m), 1.96-2.02 (2H, m), 2.42-2.47 (2H, m), 2.50-2.56 (1H, m), 3.29-3.38 (4H, m), 4.31 (2H, q), 4.46-4.51 (2H, m), 4.96 (2H, s), 8.36 (1H, s).

MSm/z: 471 (M+1).

### (d) 6-[4-(tert-Butoxycarbonyl)piperidin-1-yl]-5-cyano-2-[(2-oxopiperidin-1-yl)methyl]nicotinic acid

Ethyl 6-[4-(tert-butoxycarbonyl)piperidin-1-yl]-5-cyano-2-[(2-oxopiperidin-1-yl)methyl]nicotinate (1.11 g, 2.36 mmol) was added to a solution of NaOH (5 mL, 2 M) in MeCN (30 mL), the reaction mixture was refluxed for 1.5 h. The organic solvent was concentrated in vacuo, water was added followed by citric acid to pH 3. The precipitate was filtered of and the solid material was dried to give 6-[4-(tert-butoxycarbonyl)piperidin-1-yl]-5-cyano-2-[(2-oxopiperidin-1-yl)methyl]nicotinic acid. Yield: 0.78 g (75%).

MSm/z: 443 (M+1), 441 (M−1).

### (f) tert-Butyl 1-{3-cyano-5-(3-ethoxy-3-oxopropanoyl)-6-[(2-oxopiperidin-1-yl)methyl]pyridin-2-yl}piperidine-4-carboxylate

N,N′-Carbonyldiimidazole (0.57 g, 3.5 mmol) was added to a solution of 6-[4-(tert-butoxycarbonyl)piperidin-1-yl]-5-cyano-2-[(2-oxopiperidin-1-yl)methyl]nicotinic acid (1.1 g, 2.44 mmol) in MeCN (10 mL), the reaction mixture was stirred at 50° C. for 30 min. The reaction was cooled to 0° C. and ethyl potassium malonate (0.63 g, 3.7 mmol) and magnesium chloride, anhydrous, (0.32 g, 3.36 mmol) were added, the reaction mixture was stirred at r.t for 1 h and the refluxed for 2 h. After cooling, 1M HCl and DCM was added, the organic solvent was separated and dried (phase separator) and concentrated in vacuo to give tert-butyl 1-{3-cyano-5-(3-ethoxy-3-oxopropanoyl)-6-[(2-oxopiperidin-1-yl)methyl]pyridin-2-yl}piperidine-4-carboxylate. The crude material was used in the next step without further purification.

MSm/z: 513 (M+1), 511 (M−1)

### (g) 1-{5-Acetyl-3-cyano-6-[(2-oxopiperidin-1-yl)methyl]pyridin-2-yl}piperidine-4-carboxylic acid

tert-Butyl 1-{3-cyano-5-(3-ethoxy-3-oxopropanoyl)-6-[(2-oxopiperidin-1-yl)methyl]pyridin-2-yl}piperidine-4-carboxylate (1.25 g, 2.44 mmol) was added to a solution of formic acid (10 mL) and water (5 mL). Sulfuric acid (0.5 mL) was added dropwise and the reaction mixture was heated at 100° C. for 30 min. Water and EtOAc were added, the organic phase was separated, dried (MgSO₄) and concentrated in vacuo. Water and MeCN were added to the residue and the mixture was cooled over night, the participate was filtered and dried to give 1-{5-acetyl-3-cyano-6-[(2-oxopiperidin-1-yl)methyl]pyridin-2-yl}piperidine-4-carboxylic acid. Yield: 0.21 g (22%).

MSm/z: 385 (M+1), 383 (M−1).

### (h) 1-{5-Acetyl-3-cyano-6-[(2-oxopiperidin-1-yl)methyl]pyridin-2-yl}-N-(benzylsulfonyl)piperidine-4-carboxamide

1-{5-Acetyl-3-cyano-6-[(2-oxopiperidin-1-yl)methyl]pyridin-2-yl}piperidine-4-carboxylic acid (98 mg, 0.26 mmol) was dissolved in dry DCM (10 mL), DIPEA (0.14 mL, 0.77 mmol) and bromo-tris-pyrrolidino-phosphonium hexafluorophosphate (165 mg, 0.35 mmol) were added. The reaction mixture was stirred for 30 min at rt, 1-phenylmethanesulfonamide (61 mg, 0.36 mmol) was added and the reaction was stirred over night at rt. HCl (20 mL, 1M in water) was added, the phases were separated and the organic solvent was concentrated in vacuo and purified by HPLC (Kromasil C8 using an increasing gradient of MeCN in HOAc (aq, 0.2%)) to give 1-{5-Acetyl-3-cyano-6-[(2-oxopiperidin-1-yl)methyl]pyridin-2-yl}-N-(benzylsulfonyl)piperidine-4-carboxamide. Yield: 26 mg (19%).

¹H NMR (500 MHz, CDCl₃) δ 1.70-1.86 (8H, m), 2.28 (2H, t), 2.48-2.55 (4H, m), 3.07-3.11 (1H, m), 3.17-3.22 (2H, m), 3.32 (2H, t), 4.45-4.50 (2H, m), 4.61 (2H, s), 4.86 (2H, s), 7.31-7.38 (5H, m), 8.20 (1H, s).

MSm/z: 496 (M+1), 494 (M−1).

GTPγS(IC₅₀ μM): 1.654

### Example 4

### 1-{5-Butyryl-3-cyano-6-[(2-oxopiperidin-1-yl)methyl]pyridin-2-yl}-N-[(4-methylbenzyl)sulfonyl]piperidine-4-carboxamide

### (a) tert-Butyl 1-{5-butyryl-3-cyano-6-[(2-oxopiperidin-1-yl)methyl]pyridin-2-yl}piperidine-4-carboxylate

6-[4-(tert-Butoxycarbonyl)piperidin-1-yl]-5-cyano-2-[(2-oxopiperidin-1-yl)methyl]nicotinic acid (Example 3 (d)) (0.2 g, 0.45 mmol) was dissolved in dry DCM (5 mL) and cooled to 0° C., oxalyl chloride (0.12 mL, 1.26 mmol) and 3 drops of dry DMF were added and the reaction was stirred at 0° C. for 30 min. The solvent was concentrated in vacuo and the residue was dissolved in dry THF (5 mL), ferric acetylacetonate (48 mg, 0.136 mmol) was added followed by dropwise addition of propyl magnesium chloride (0.79 mL, 1.58 mmol, 2M in dimethylether). The reaction was stirred at r.t 2 h, water was added and the organic solvent was separated and dried (phase separator) and concentrated in vacuo. The residue was diluted with water and extracted with EtOAc (2×150 mL). The combined organic phases was dried (Na₂SO₄), filtered and concentrated in vacuo. The crude product was purified by HPLC (Kromasil C8, 250×50 ID mm, using a gradient of 40% to 95% MeCN over 30 minutes with an acidic second eluent (H₂O/MeCN/FA, 95/5/0.2)). The organic solvent was concentrated in vacuo and the water phase was diluted with water and made basic (pH=9) with sat. NaHCO₃ (aq), the water phase was extracted with EtOAc (2×150 mL). The combined organic phases were dried (Na₂SO₄), filtered and concentrated in vacuo to give tert-butyl 1-{5-butyryl-3-cyano-6-[(2-oxopiperidin-1-yl)methyl]pyridin-2-yl}piperidine-4-carboxylate. Yield: 64 mg (14%).

### (b) 1-{5-Butyryl-3-cyano-6-[(2-oxopiperidin-1-yl)methyl]pyridin-2-yl}piperidine-4-carboxylic acid

tert-Butyl 1-{5-butyryl-3-cyano-6-[(2-oxopiperidin-1-yl)methyl]pyridin-2-yl}piperidine-4-carboxylate (30 mg, 0.064 mmol) was dissolved in DCM (2 mL), TFA (1 mL, 12 mmol) was added. The reaction mixture was stirred at r.t for 1 h, the solvent was concentrated in vacuo and the crude product was coevaporated with DCM (3×). The crude product was used in the next step without purification.

MSm/z: 413 (M+1), 411 (M−1).

### (c) 1-{5-Butyryl-3-cyano-6-[(2-oxopiperidin-1-yl)methyl]pyridin-2-yl}-N-[(4-methylbenzyl)sulfonyl]piperidine-4-carboxamide

Prepared according to Example 3(h) from 1-{5-Butyryl-3-cyano-6-[(2-oxopiperidin-1-yl)methyl]pyridin-2-yl}piperidine-4-carboxylic acid (26.4 mg, 0.06 mmol) by using 1-(4-methylphenyl)methanesulfonamide in place of 1-phenylmethanesulfonamide to give 1-{5-butyryl-3-cyano-6-[(2-oxopiperidin-1-yl)methyl]pyridin-2-yl}-N-[(4-methylbenzyl)sulfonyl]piperidine-4-carboxamide. Yield: 9.2 mg (25%).

¹H NMR (500 MHz, DMSO-d₆) δ 0.92 (3H, t), 1.55-1.65 (4H, m), 1.75-1.85 (6H, m), 2.25-2.28 (2H, m), 2.31 (3H, s), 2.57-2.62 (1H, m), 2.92 (2H, t), 3.18-3.24 (2H, m), 3.32-3.35 (2H, m), 4.52-4.56 (2H, m), 4.63 (2H, s), 4.73 (2H, s), 7.16-7.22 (4H, m), 8.63 (1H, s), 11.53 (1H, s).

MSm/z: 580 (M+1), 578 (M−1).

GTPγS(IC₅₀ μM): 0.021

### Example 5

### 1-{5-butyryl-3-cyano-6-[(2-oxopyrrolidin-1-yl)methyl]pyridin-2-yl}-N-[(4-methylbenzyl)sulfonyl]piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (Example 2 (b)) (96.4 mg, 0.24 mmol) by using 1-(4-methylphenyl)methanesulfonamide in place of 1-phenylmethanesulfonamide to give 1-{5-butyryl-3-cyano-6-[(2-oxopyrrolidin-1-yl)methyl]pyridin-2-yl}-N-[(4-methylbenzyl)sulfonyl]piperidine-4-carboxamide. Yield: 79.5 mg (58%).

¹H-NMR (500 MHz, DMSO-d₆) δ 0.92 (3H, t), 1.55-1.65 (4H, m), 1.81-1.85 (2H, m), 2.00 (2H, quintet), 2.27-2.32 (5H, m), 2.58-2.64 (1H, m), 2.93 (2H, t), 3.17-3.23 (2H, m), 3.43 (2H, t), 4.48-4.52 (2H, m), 4.63 (2H, s), 4.68 (2H, s), 7.15-7.22 (4H, m), 8.64 (1H, s), 11.54 (1H, s).

MSm/z: 566 (M+1), 564 (M−1).

GTPγS(IC₅₀ μM): 0.009

### Example 6

### 1-{5-butyryl-3-cyano-6-[(2-oxopyrrolidin-1-yl)methyl]pyridin-2-yl}-N-[(2,4-difluorobenzyl)sulfonyl]piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (Example 2(b)) (96.4 mg, 0.24 mmol) by using 1-(2,4-difluorophenyl)methanesulfonamide in place of 1-phenylmethanesulfonamide to give 1-{5-butyryl-3-cyano-6-[(2-oxopyrrolidin-1-yl)methyl]pyridin-2-yl}-N-[(2,4-difluorobenzyl)sulfonyl]piperidine-4-carboxamide. Yield: 65 mg (46%).

¹H NMR (500 MHz, DMSO-d₆) δ 0.92 (3H, t), 1.55-1.65 (4H, m), 1.84-1.89 (2H, m), 2.00 (1H, quintet), 2.28 (2H, t), 2.60-2.66 (2H, m), 2.93 (2H, t), 3.18-3.24 (2H, m), 3.43 (2H, t), 4.51 (2H, d), 4.68 (2H, s), 4.74 (2H, s), 7.16-7.20 (1H, m), 7.32-7.36 (1H, m), 7.43-7.49 (1H, m), 8.64 (1H, s), 11.74 (1H, br s).

MSm/z: 588 (M+1), 586 (M−1).

GTPγS(IC₅₀ μM): 0.0099

### Example 7

### 1-{5-Butyryl-3-cyano-6-[(2-oxopyrrolidin-1-yl)methyl]pyridin-2-yl}-N-[(cyclopentylmethyl)sulfonyl]piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (Example 2(b)) (390 mg, 0.98 mmol) by using 1-cyclopentylmethanesulfonamide in place of 1-phenylmethanesulfonamide to give 1-{5-butyryl-3-cyano-6-[(2-oxopyrrolidin-1-yl)methyl]pyridin-2-yl}-N-[(cyclopentylmethyl)sulfonyl]piperidine-4-carboxamide. Yield: 374 mg (70%).

¹H NMR (500 MHz, DMSO-d₆) δ 0.91 (3H, t), 1.20-1.28 (2H, m), 1.44-1.63 (8H, m), 1.82-1.92 (4H, m), 1.98 (2H, quintet), 2.10-2.18 (1H, m), 2.27 (2H, t), 2.65-2.71 (1H, m), 2.92 (2H, t), 3.19-3.26 (2H, m), 3.40-3.43 (4H, m), 4.46-4.51 (2H, m), 4.67 (2H, s), 8.63 (1H, s), 11.70 (1H, s).

MSm/z: 544 (M+1), 542 (M−1)

GTPγS(IC₅₀ μM): 0.028

### Example 8

### N-(benzylsulfonyl)-1-{3-cyano-5-(cyclopropylcarbonyl)-6-[(2-oxopyrrolidin-1-yl)methyl]pyridin-2-yl}piperidine-4-carboxamide

### (a) tert-Butyl 1-{3-cyano-5-(cyclopropylcarbonyl)-6-[(2-oxopyrrolidin-1-yl)methyl]pyridin-2-yl}piperidine-4-carboxylate

Prepared according to Example 1(g) from tert-butyl 1-{5-(chlorocarbonyl)-3-cyano-6-[(2-oxopyrrolidin-1-yl)methyl]pyridin-2-yl}piperidine-4-carboxylate

(Example 1(e)) (1 g, 2.33 mmol) by using cyclopropyl magnesium bromide in place of methylmagnesium chloride to give tert-butyl 1-{3-cyano-5-(cyclopropylcarbonyl)-6-[(2-oxopyrrolidin-1-yl)methyl]pyridin-2-yl}piperidine-4-carboxylate. This reaction was performed at 0° C. Yield: 0.27 g (25%).

¹H NMR (500 MHz, CDCl₃) δ 1.01-1.05 (1H, m), 1.16-1.20 (1H, m), 1.45 (9H, s), 1.74-1.85 (3H, m), 1.94-2.11 (5H, m), 2.33-2.39 (1H, m), 2.41-2.46 (1H, m), 2.49-2.59 (1H, m), 3.26-3.37 (2H, m), 3.45-3.49 (1H, m), 3.57-3.65 (1H, m), 4.26-4.29 (2H, m), 4.40-4.51 (2H, m), 4.80 (1H, s), 8.34 (1H, s).

### (b) 1-{3-Cyano-5-(cyclopropylcarbonyl)-6-[(2-oxopyrrolidin-1-yl)methyl]pyridin-2-yl}piperidine-4-carboxylic acid

Prepared according to Example 1(h) from tert-butyl 1-{3-cyano-5-(cyclopropylcarbonyl)-6-[(2-oxopyrrolidin-1-yl)methyl]pyridin-2-yl}piperidine-4-carboxylate (0.69 g, 1.52 mmol) to give 1-{3-cyano-5-(cyclopropylcarbonyl)-6-[(2-oxopyrrolidin-1-yl)methyl]pyridin-2-yl}piperidine-4-carboxylic acid. Yield: 1.5 g (99%).

MSm/z: 397 (M+1), 395 (M−1).

### (c) N-(Benzylsulfonyl)-1-{3-cyano-5-(cyclopropylcarbonyl)-6-[(2-oxopyrrolidin-1-yl)methyl]pyridin-2-yl}piperidine-4-carboxamide

Prepared according to Example 1(i) from 1-{3-cyano-5-(cyclopropylcarbonyl)-6-[(2-oxopyrrolidin-1-yl)methyl]pyridin-2-yl}piperidine-4-carboxylic acid (109 mg, 0.28 mmol) to give N-(Benzylsulfonyl)-1-{3-cyano-5-(cyclopropylcarbonyl)-6-[(2-oxopyrrolidin-1-yl)methyl]pyridin-2-yl}piperidine-4-carboxamide. Yield: 39 mg (26%).

¹H-NMR (500 MHz, DMSO-d₆) δ 0.99-1.02 (3H, m), 1.26 (1H, t), 1.57-1.74 (2H, m), 1.80-1.86 (2H, m), 1.99 (2H, quintet), 2.28 (2H, t), 2.58-2.64 (1H, m), 2.77-2.82 (1H, m), 3.18-3.23 (2H, m), 3.42 (2H, t), 4.49-4.53 (2H, m), 4.65 (2H, s), 4.70 (2H, s), 7.28-7.31 (2H, m), 7.38-7.43 (3H, m), 8.80 (1H, s), 11.60 (1H, br s).

MSm/z: 550 (M+1), 548 (M−1).

GTPγS(IC₅₀ μM): 0.032

### Example 9

### 1-{3-Cyano-5-(cyclopropylcarbonyl)-6-[(2-oxopyrrolidin-1-yl)methyl]pyridin-2-yl}-N-[(cyclopentylmethyl)sulfonyl]piperidine-4-carboxamide

Prepared according to Example 1(i) from 1-{3-cyano-5-(cyclopropylcarbonyl)-6-[(2-oxopyrrolidin-1-yl)methyl]pyridin-2-yl}piperidine-4-carboxylic acid (Example 8(b)) (109 mg, 0.28 mmol) by using 1-cyclopentylmethanesulfonamide in place of 1-phenylmethanesulfonamide to give 1-{3-cyano-5-(cyclopropylcarbonyl)-6-[(2-oxopyrrolidin-1-yl)methyl]pyridin-2-yl}-N-[(cyclopentylmethyl)sulfonyl]piperidine-4-carboxamide. Yield: 21 mg (14%).

¹H-NMR (500 MHz, DMSO-d₆) δ 0.98-1.01 (4H, m), 1.20-1.28 (2H, m), 1.46-1.53 (2H, m), 1.55-1.63 (4H, m), 1.82-1.93 (4H, m), 1.98 (2H, quintet), 2.10-2.19 (1H, m), 2.27 (2H, t), 2.66-2.72 (1H, m), 2.76-2.81 (1H, m), 3.20-3.26 (2H, m), 3.40-3.42 (4H, m), 4.48-4.52 (2H, m), 4.64 (2H, s), 8.79 (1H, s), 11.70 (1H, s).

MSm/z: 542 (M+1), 540 (M−1).

GTPγS(IC₅₀ μM): 0.058

### Example 10

### N-(benzylsulfonyl)-1-{3-cyano-6-[(2-oxopyrrolidin-1-yl)methyl]-5-propionylpyridin-2-yl}piperidine-4-carboxamide

### (a) tert-Butyl 1-{3-cyano-6-[(2-oxopyrrolidin-1-yl)methyl]-5-propionylpyridin-2-yl}piperidine-4-carboxylate

Prepared according to Example 1(g) from tert-butyl 1-{5-(chlorocarbonyl)-3-cyano-6-[(2-oxopyrrolidin-1-yl)methyl]pyridin-2-yl}piperidine-4-carboxylate (Example 1(e)) (1.56 g, 3.64 mmol) by using ethylmagnesium bromide in place of methylmagnesium chloride to give tert-butyl 1-{3-cyano-6-[(2-oxopyrrolidin-1-yl)methyl]-5-propionylpyridin-2-yl}piperidine-4-carboxylate. Yield: 0.34 g (21%).

¹H-NMR (500 MHz, CDCl₃) δ 0.96 (1H, t), 1.14 (1H, t), 1.42 (9H, s), 1.71-1.83 (2H, m), 1.86-1.92 (1H, m), 1.94-2.01 (2H, m), 2.03-2.09 (1H, m), 2.35-2.54 (4H, m), 2.79-2.84 (1H, m), 3.22-3.34 (2H, m), 3.44-3.48 (1H, m), 3.52-3.56 (1H, m), 4.25 (1H, s), 4.37-4.49 (2H, m), 4.82 (2H, s), 8.18 (1H, s).

### (b) 1-{3-Cyano-6-[(2-oxopyrrolidin-1-yl)methyl]-5-propionylpyridin-2-yl}piperidine-4-carboxylic acid

Prepared according to Example 1(h) from tert-butyl 1-{3-cyano-6-[(2-oxopyrrolidin-1-yl)methyl]-5-propionylpyridin-2-yl}piperidine-4-carboxylate (340 mg, 0.77 mmol) to give 1-{3-cyano-6-[(2-oxopyrrolidin-1-yl)methyl]-5-propionylpyridin-2-yl}piperidine-4-carboxylic acid. Yield: 296 mg (100%).

MSm/z: 385 (M+1), 383 (M−1).

### (c) N-(Benzylsulfonyl)-1-{3-cyano-6-[(2-oxopyrrolidin-1-yl)methyl]-5-propionylpyridin-2-yl}piperidine-4-carboxamide

Prepared according to Example 1(i) from tert-butyl 1-{3-cyano-6-[(2-oxopyrrolidin-1-yl)methyl]-5-propionylpyridin-2-yl}piperidine-4-carboxylate (99 mg, 0.26 mmol) to give N-(benzylsulfonyl)-1-{3-cyano-6-[(2-oxopyrrolidin-1-yl)methyl]-5-propionylpyridin-2-yl}piperidine-4-carboxamide. Yield: 36 mg (26%).

¹H-NMR (400 MHz, DMSO-d₆) δ 0.99 (3H, t), 1.50-1.84 (6H, m), 1.96 (2H, pentet), 2.25 (2H, t), 2.50-2.61 (1H, m), 2.92 (2H, q), 3.11-3.20 (2H, m), 4.41-4.49 (2H, m), 4.64-4.66 (4H, m), 7.23-7.28 (2H, m), 7.33-7.40 (3H, m), 8.58 (1H, s), 11.56 (1H, br s).

MSm/z: 538 (M+1), 536 (M−1)

GTPγS(IC₅₀ μM): 0.098

### Example 11

### 1-{3-cyano-6-[(2-oxopyrrolidin-1-yl)methyl]-5-propionylpyridin-2-yl}-N-[(4-methylbenzyl)sulfonyl]piperidine-4-carboxamide

Prepared according to Example 1(i) from 1-{3-cyano-6-[(2-oxopyrrolidin-1-yl)methyl]-5-propionylpyridin-2-yl}piperidine-4-carboxylic acid (Example 10(b)) (99 mg, 0.26 mmol) by using 1-(4-methylphenyl)methanesulfonamide in place of 1-phenylmethanesulfonamide to give 1-{3-cyano-6-[(2-oxopyrrolidin-1-yl)methyl]-5-propionylpyridin-2-yl}-N-[(4-methylbenzyl)sulfonyl]piperidine-4-carboxamide. Yield: 110 mg (78%).

¹H-NMR (400 MHz, DMSO-d₆) δ 1.00 (3H, t), 1.50-1.65 (2H, m), 1.75-1.84 (2H, m), 1.96 (2H, pentet), 2.25 (2H, t), 2.27 (3H, s), 2.51-2.62 (1H, m), 2.92 (2H, q), 3.11-3.21 (2H, m), 3.39 (2H, t), 4.42-4.50 (2H, m), 4.59 (2H, s), 4.64 (2H, s), 7.10-7.19 (4H, m), 8.58 (1H, s), 11.51 (1H, br s).

MSm/z: 552 (M+1), 550 (M−1).

GTPγS(IC₅₀ μM): 0.037

### Example 12

### 1-{3-Cyano-6-[(2-oxopyrrolidin-1-yl)methyl]-5-propionylpyridin-2-yl}-N-[(cyclopentylmethyl)sulfonyl]piperidine-4-carboxamide

Prepared according to Example 1(i) from 1-{3-cyano-6-[(2-oxopyrrolidin-1-yl)methyl]-5-propionylpyridin-2-yl}piperidine-4-carboxylic acid (Example 10(b)) (99 mg, 0.26 mmol) by using 1-cyclopentylmethanesulfonamide in place of 1-phenylmethanesulfonamide to give 1-{3-cyano-6-[(2-oxopyrrolidin-1-yl)methyl]-5-propionylpyridin-2-yl}-N-[(cyclopentylmethyl)sulfonyl]piperidine-4-carboxamide. Yield: 53 mg (39%).

¹H-NMR (400 MHz, DMSO-d₆) δ 0.99 (3H, t), 1.14-1.25 (2H, m), 1.40-1.64 (6H, m), 1.76-1.89 (4H, m), 1.94 (2H, pentet), 2.05-2.16 (1H, m), 2.23 (2H, t), 2.57-2.69 (1H, m), 2.92 (2H, q), 3.14-3.22 (2H, m), 3.34-3.41 (4H, m), 4.41-4.48 (2H, m), 4.63 (2H, s), 8.57 (1H, s), 11.67 (1H, br s).

MSm/z: 530 (M+1), 528 (M−1).

GTPγS(IC₅₀ μM): 0.328

### Example 13

### 1-{3-Cyano-5-(cyclopropylcarbonyl)-6-[(2-oxopiperidin-1-yl)methyl]pyridin-2-yl}-N-{[methyl(phenyl)amino]sulfonyl}piperidine-4-carboxamide

### (a) tert-Butyl 1-{3-cyano-5-(cyclopropylcarbonyl)-6-[(2-oxopiperidin-1-yl)methyl]pyridin-2-yl}piperidine-4-carboxylate

6-[4-(tert-Butoxycarbonyl)piperidin-1-yl]-5-cyano-2-[(2-oxopiperidin-1-yl)methyl]nicotinic acid (Example 3 (d)) (1.33 g, 3 mmol) was dissolved in dry DCM (50 mL) and cooled to 0° C., oxalyl chloride (0.76 mL, 9 mmol) and 3 drops of dry DMF were added and the reaction was stirred at 0° C. for 30 min. The solvet was concentrated in vacuo and the residue was dissolved in dry THF (50 mL), ferric acetylacetonate (32 mg, 0.09 mmol) was added followed by dropwise addition of cyclopropyl magnesium bromide (15 mL, 8 mmol, 0.5 M in THF). The reaction was stirred at r.t 2 h, water (3 mL) was added and the organic solvent was separated and dried (phase separator) and concentrated in vacuo. The residue was diluted with water and extracted with EtOAc (2×150 mL). The combined organic phases was dried (Na₂SO₄), filtered and concentrated in vacuo. The crude product was purified by HPLC (Kromasil C8, 10 um, 250×50 ID mm, using a gradient of 40% to 95% MeCN over 30 minutes with an acidic second eluent (H₂O/MeCN/FA, 95/5/0.2). The organic solvent was concentrated in vacuo and the water phase was diluted with water and made basic (pH=9) with sat. NaHCO₃ (aq), the water phase was extracted with EtOAc (2×150 mL). The combined organic phases were dried (Na₂SO₄), filtered and concentrated in vacuo to give tert-butyl 1-{3-cyano-5-(cyclopropylcarbonyl)-6-[(2-oxopiperidin-1-yl)methyl]pyridin-2-yl}piperidine-4-carboxylate. Yield: 0.28 g (20%).

¹H-NMR (500 MHz, CDCl₃) δ 0.99-1.03 (2H, m), 1.15-1.19 (2H, m), 1.44 (9H, s), 1.72-1.87 (6H, m), 1.96-2.02 (2H, m), 2.34-2.44 (3H, m), 2.50-2.56 (1H, m), 3.25-3.38 (4H, m), 4.46-4.53 (2H, m), 4.84 (2H, s), 8.33 (1H, s).

MSm/z: 467 (M+1).

### (b) 1-{3-Cyano-5-(cyclopropylcarbonyl)-6-[(2-oxopiperidin-1-yl)methyl]pyridin-2-yl}piperidine-4-carboxylic acid

tert-Butyl 1-{3-cyano-5-(cyclopropylcarbonyl)-6-[(2-oxopiperidin-1-yl)methyl]pyridin-2-yl}piperidine-4-carboxylate (276 mg, 0.59 mmol) was dissolved in dry DCM:TFA 1:1 (5 mL). The reaction mixture was stirred at r.t for 1 h, the solvent was concentrated in vacuo and the crude product was coevaporated with DCM (3×). The crude product was used in the next step without purification.

MSm/z: 411 (M+1).

### (c) N-Methyl-N-phenylsulfamide

Chlorosulfonyl isocyanate (3.7 mL, 42.4 mmol) was dissolved in dry DCM (40 mL), the solution was cooled to 0° C. and tert-butanol (3.98 mL, 42.4 mmol) was added dropwise. The reaction mixture was stirred at r.t for 2 h, the solution was cooled to 0° C. and N-methylaniline (4.61 mL, 42.4 mmol) and TEA (8.85 mL, 63.6 mmol) dissolved in dry DCM (20 mL) were added dropwise through a dropping funnel. The reaction was stirred at r.t for 3 h, water was added and the organic phase was separated and dried (phase separator, Isolute) and concentrated in vacuo. The residue was dissolved in DCM (40 mL) and trifluoroacetic acid (32.7 mL, 423 mmol) was added. The reaction was stirred at r.t for 20 min, the solvent was concentrated in vacuo and coevaporated with DCM (3×). The crude product was purified with flash column chromatography, using a mixture of heptane:EtOAc 70:30 as eluent, to give N-methyl-N-phenylsulfamide. Yield: 5.96 g (76%).

¹H-NMR (500 MHz, CDCl₃) δ 3.22 (3H, s), 4.77 (2H, s), 7.28-7.33 (1H, m), 7.36-7.42 (4H, m).

MSm/z: 187 (M+1).

### (d) 1-{3-Cyano-5-(cyclopropylcarbonyl)-6-[(2-oxopiperidin-1-yl)methyl]pyridin-2-yl}-N-{[methyl(phenyl)amino]sulfonyl}piperidine-4-carboxamide

1-{3-Cyano-5-(cyclopropylcarbonyl)-6-[(2-oxopiperidin-1-yl)methyl]pyridin-2-yl}piperidine-4-carboxylic acid (Example 13 (b)) (122 mg, 0.3 mmol) was dissolved in dry DCM (4 mL), TBTU (124 mg, 0.4 mmol) and DIPEA (0.26 mL, 1.5 mmol) were added. The reaction was stirred at r.t for 1 h, N-methyl-N-phenylsulfamide (66 mg, 0.4 mmol) was added and the reaction mixture was stirred at r.t over night (16 h). The solution was diluted with DCM and washed with sat. NaHCO₃ (aq), the organic phase was dried (phase separator) and concentrated in vacuo. The residue was dissolved in DMSO (3 mL) and purified by HPLC (Kromasil C8, 10 um, 250×50 ID mm, using a gradient of 40% to 95% MeCN over 30 minutes with an acidic second eluent (H₂O/MeCN/FA, 95/5/0.2). The organic solvent was concentrated in vacuo and the water phase was diluted with water and made basic (pH=8) with sat. NaHCO₃ (aq), the water phase was extracted with DCM (100 ml) and dried (phase separator, Isolute) and concentrated in vacuo to give 1-{3-Cyano-5-(cyclopropylcarbonyl)-6-[(2-oxopiperidin-1-yl)methyl]pyridin-2-yl}-N-{[methyl(phenyl)amino]sulfonyl}piperidine-4-carboxamide. Yield: 94 mg (55%).

¹H-NMR (500 MHz, CDCl₃) δ 1.05-1.01 (2H, m), 1.19-1.15 (2H, m), 1.86-1.66 (8H, m), 2.28-2.25 (2H, m), 2.43-2.38 (1H, m), 2.50-2.44 (1H, m), 3.15-3.09 (2H, m), 3.34-3.30 (2H, m), 3.45 (3H, s), 4.49-4.42 (2H, m), 4.82 (2H, s), 7.29-7.25 (1H, m), 7.37-7.31 (4H, m), 8.38 (1H, s).

MSm/z: 579 (M+1).

GTPγS(IC₅₀ μM): 0.153

### Example 14

### 1-{3-Cyano-5-(cyclopropylcarbonyl)-6-[(2-oxopiperidin-1-yl)methyl]pyridin-2-yl}-N-[(cyclopentylmethyl)sulfonyl]piperidine-4-carboxamide

Prepared according to Example 13(d) from 1-{3-cyano-5-(cyclopropylcarbonyl)-6-[(2-oxopiperidin-1-yl)methyl]pyridin-2-yl}piperidine-4-carboxylic acid (Example 13(b)) (122 mg, 0.3 mmol) by using 1-cyclopentylmethanesulfonamide (58 mg, 0.36 mmol) in place of N-methyl-N-phenylsulfamide to give 1-{3-cyano-5-(cyclopropylcarbonyl)-6-[(2-oxopiperidin-1-yl)methyl]pyridin-2-yl}-N-[(cyclopentylmethyl)sulfonyl]piperidine-4-carboxamide. Yield: 80 mg (48%).

¹H-NMR (500 MHz, CDCl₃) δ 1.02-1.07 (2H, m), 1.16-1.20 (2H, m), 1.24-1.32 (2H, m), 1.54-1.60 (2H, m), 1.62-1.70 (2H, m), 1.76-1.98 (10H, m), 2.27-2.34 (1H, m), 2.39-2.45 (1H, m), 2.46-2.49 (2H, m), 2.59-2.66 (1H, m), 3.19-3.26 (2H, m), 3.33-3.37 (2H, m), 3.44 (2H, d), 4.47-4.54 (2H, m), 4.88 (2H, s), 8.42 (1H, s), 10.36-10.59 (1H, m).

MSm/z: 556 (M+1).

GTPγS(IC₅₀ μM): 0.461

### Example 15

### N-(Benzylsulfonyl)-1-{3-cyano-5-isobutyryl-6-[(2-oxopyrrolidin-1-yl)methyl]pyridin-2-yl}piperidine-4-carboxamide

### (a) tert-Butyl 1-{3-cyano-5-isobutyryl-6-[(2-oxopyrrolidin-1-yl)methyl]pyridin-2-yl}piperidine-4-carboxylate

Prepared according to Example 1(g) from tert-butyl 1-{5-(chlorocarbonyl)-3-cyano-6-[(2-oxopyrrolidin-1-yl)methyl]pyridin-2-yl}piperidine-4-carboxylate (Example 1 (e)) (610 mg, 1.34 mmol) by using isopropylmagnesium bromide in place of methylmagnesium chloride to give tert-butyl 1-{3-cyano-5-isobutyryl-6-[(2-oxopyrrolidin-1-yl)methyl]pyridin-2-yl}piperidine-4-carboxylate. Yield: 0.61 g (12%).

¹H-NMR (500 MHz, CDCl₃) δ 1.05-1.08 (1H, m), 1.19 (4H, d), 1.47 (9H, s), 1.76-1.85 (2H, m), 1.98-2.14 (5H, m), 2.44-2.48 (2H, m), 2.53-2.60 (1H, m), 3.30-3.39 (3H, m), 3.47-3.51 (2H, m), 4.48-4.53 (2H, m), 4.85 (2H, s), 8.18 (1H, s).

MSm/z: 455 (M+1).

### (b) 1-{3-Cyano-5-isobutyryl-6-[(2-oxopyrrolidin-1-yl)methyl]pyridin-2-yl}piperidine-4-carboxylic acid

Prepared according to Example 1(h) from tert-butyl 1-{3-cyano-5-isobutyryl-6-[(2-oxopyrrolidin-1-yl)methyl]pyridin-2-yl}piperidine-4-carboxylate (610 mg, 1.34 mmol) to give 1-{3-cyano-5-isobutyryl-6-[(2-oxopyrrolidin-1-yl)methyl]pyridin-2-yl}piperidine-4-carboxylic acid. Yield: 0.53 g (99%).

MSm/z: 399 (M+1).

### (c) N-(Benzylsulfonyl)-1-{3-cyano-5-isobutyryl-6-[(2-oxopyrrolidin-1-yl)methyl]pyridin-2-yl}piperidine-4-carboxamide

Prepared according to Example 1(i) from 1-{3-cyano-5-isobutyryl-6-[(2-oxopyrrolidin-1-yl)methyl]pyridin-2-yl}piperidine-4-carboxylic acid (265 mg, 0.67 mmol) to give N-(benzylsulfonyl)-1-{3-cyano-5-isobutyryl-6-[(2-oxopyrrolidin-1-yl)methyl]pyridin-2-yl}piperidine-4-carboxamide. Purification Method A was used (See General Experimental Procedure). Yield: 0.118 g (29%).

¹H-NMR (400 MHz, DMSO-d₆) δ 1.01 (6H, d), 1.49-1.83 (4H, m), 1.89-1.99 (2H, m), 2.20-2.26 (2H, m), 2.51-2.60 (1H, m), 3.08-3.20 (2H, m), 3.33-3.41 (2H, m), 3.54 (1H, quintet), 4.41-4.48 (2H, m), 4.59 (2H, s), 4.63 (2H, s), 7.21-7.39 (5H, m), 8.58 (1H, s), 11.55 (1H, s).

MSm/z: 552 (M+1), 550 (M−1).

GTPγS(IC₅₀ μM): 0.106

### Example 16

### 1-{3-Cyano-5-isobutyryl-6-[(2-oxopyrrolidin-1-yl)methyl]pyridin-2-yl}-N-[(cyclopentylmethyl)sulfonyl]piperidine-4-carboxamide

Prepared according to Example 1(i) from 1-{3-cyano-5-isobutyryl-6-[(2-oxopyrrolidin-1-yl)methyl]pyridin-2-yl}piperidine-4-carboxylic acid (Example 15(b)) (265 mg, 0.67 mmol) by using 1-cyclopentylmethanesulfonamide in place of 1-phenylmethanesulfonamide to give 1-{3-Cyano-5-isobutyryl-6-[(2-oxopyrrolidin-1-yl)methyl]pyridin-2-yl}-N-[(cyclopentylmethyl)sulfonyl]piperidine-4-carboxamide. Purification Method A was used (See General Experimental Procedure). Yield: 100 mg (25%).

¹H-NMR (400 MHz, DMSO-d₆) δ 1.00 (6H, d), 1.11-1.25 (2H, m), 1.37-1.60 (7H, m), 1.74-1.97 (7H, m), 2.03-2.14 (1H, m), 2.18-2.25 (2H, m), 2.57-2.66 (1H, m), 3.11-3.21 (2H, m), 3.31-3.39 (2H, m), 3.53 (1H, quintet), 4.39-4.47 (2H, m), 4.57 (2H, s), 8.56 (1H, s), 11.67 (1H, s).

MSm/z: 544 (M+1), 542 (M−1).

GTPγS(IC₅₀ μM): 0.396

### Example 17

### 1-{3-Cyano-6-[(2-oxopyrrolidin-1-yl)methyl]-5-pentanoylpyridin-2-yl}-N-[(cyclopentylmethyl)sulfonyl]piperidine-4-carboxamide

### (a) tert-Butyl 1-{3-cyano-6-[(2-oxopyrrolidin-1-yl)methyl]-5-pentanoylpyridin-2-yl}piperidine-4-carboxylate

Prepared according to Example 1(g) from tert-butyl 1-{5-(chlorocarbonyl)-3-cyano-6-[(2-oxopyrrolidin-1-yl)methyl]pyridin-2-yl}piperidine-4-carboxylate

(Example 1(e)) (5 g, 11.7 mmol) by using N-butylmagnesium chloride in place of methylmagnesium chloride to give tert-butyl 1-{3-cyano-6-[(2-oxopyrrolidin-1-yl)methyl]-5-pentanoylpyridin-2-yl}piperidine-4-carboxylate. Yield: 1.4 g (26%).

¹H-NMR (400 MHz, DMSO-d₆) δ 0.95 (3H, t), 1.34-1.43 (2H, m), 1.45 (9H, s), 1.62-1.69 (2H, m), 1.73-1.82 (2H, m), 1.97-2.03 (2H, m), 2.06-2.12 (2H, m), 2.45 (2H, t), 2.51-2.58 (1H, m), 2.78-2.82 (2H, m), 3.31-3.38 (2H, m), 3.47-3.50 (2H, m), 4.46-4.51 (2H, m), 4.85 (2H, s), 8.18 (1H, s).

MSm/z: 469 (M+1).

### (b) 1-{3-Cyano-6-[(2-oxopyrrolidin-1-yl)methyl]-5-pentanoylpyridin-2-yl}piperidine-4-carboxylic acid

Prepared according to Example 1(h) from tert-butyl 1-{3-cyano-6-[(2-oxopyrrolidin-1-yl)methyl]-5-pentanoylpyridin-2-yl}piperidine-4-carboxylate (1.4 g, 3 mmol) to give 1-{3-cyano-6-[(2-oxopyrrolidin-1-yl)methyl]-5-pentanoylpyridin-2-yl}piperidine-4-carboxylic acid. Yield: 1.22 g (99%).

MSm/z: 413 (M+1).

### (c) 1-{3-Cyano-6-[(2-oxopyrrolidin-1-yl)methyl]-5-pentanoylpyridin-2-yl}-N-[(cyclopentylmethyl)sulfonyl]piperidine-4-carboxamide

Prepared according to Example 1(i) from 1-{3-cyano-6-[(2-oxopyrrolidin-1-yl)methyl]-5-pentanoylpyridin-2-yl}piperidine-4-carboxylic acid (203 mg, 0.49 mmol) by using 1-cyclopentylmethanesulfonamide in place of 1-phenylmethanesulfonamide to give 1-{3-Cyano-6-[(2-oxopyrrolidin-1-yl)methyl]-5-pentanoylpyridin-2-yl}-N-[(cyclopentylmethyl)sulfonyl]piperidine-4-carboxamide. Purification Method A was used (See General Experimental Procedure). Yield: 69.7 mg (25%).

¹H-NMR (400 MHz, DMSO-d₆) δ 0.89 (3H, t), 1.17-1.37 (4H, m), 1.43-1.64 (9H, m), 1.79-1.92 (5H, m), 1.92-2.02 (2H, m), 2.08-2.18 (1H, m), 2.22-2.29 (2H, m), 2.63-2.72 (1H, m), 2.93 (2H, t), 3.16-3.26 (2H, m), 3.36-3.43 (2H, m), 4.43-4.52 (2H, m), 4.65 (2H, s), 8.63 (1H, s), 11.70 (1H, s).

MSm/z: 558 (M+1).

GTPγS(IC₅₀ μM): 0.029

### Example 18

### N-(Benzylsulfonyl)-1-{3-cyano-6-[(2-oxopyrrolidin-1-yl)methyl]-5-pentanoylpyridin-2-yl}piperidine-4-carboxamide

Prepared according to Example 1(i) from 1-{3-cyano-6-[(2-oxopyrrolidin-1-yl)methyl]-5-pentanoylpyridin-2-yl}piperidine-4-carboxylic acid (Example 17(b)) (203 mg, 0.49 mmol) to give N-(benzylsulfonyl)-1-{3-cyano-6-[(2-oxopyrrolidin-1-yl)methyl]-5-pentanoylpyridin-2-yl}piperidine-4-carboxamide. Purification Method A was used (See General Experimental Procedure). Yield: 117 mg (42%).

¹H-NMR (400 MHz, DMSO-d₆) δ 0.89 (3H, t), 1.32 (2H, sextet), 1.48-1.65 (4H, m), 1.77-1.85 (2H, m), 1.93-2.03 (2H, m), 2.24-2.31 (2H, m), 2.54-2.63 (1H, m), 2.93 (2H, t), 3.13-3.23 (2H, m), 3.38-3.45 (2H, m), 4.44-4.52 (2H, m), 4.66 (2H, s), 4.68 (2H, s), 7.22-7.31 (2H, m), 7.35-7.43 (3H, m), 8.63 (1H, s), 11.59 (1H, s).

MSm/z: 566 (M+1).

GTPγS(IC₅₀ μM): 0.013

### Example 19

### 1-{3-Cyano-6-[(2-oxopyrrolidin-1-yl)methyl]-5-pentanoylpyridin-2-yl}-N-[(4-methylbenzyl)sulfonyl]piperidine-4-carboxamide

Prepared according to Example 1(i) from 1-{3-cyano-6-[(2-oxopyrrolidin-1-yl)methyl]-5-pentanoylpyridin-2-yl}piperidine-4-carboxylic acid (Example 17(b)) (203 mg, 0.49 mmol) by using 1-(4-methylphenyl)methanesulfonamide in place of 1-phenylmethanesulfonamide to give 1-{3-Cyano-6-[(2-oxopyrrolidin-1-yl)methyl]-5-pentanoylpyridin-2-yl}-N-[(4-methylbenzyl)sulfonyl]piperidine-4-carboxamide. Purification Method A was used (See General Experimental Procedure). Yield: 85 mg (29%).

¹H-NMR (400 MHz, DMSO-d₆) δ 0.85 (3H, t), 1.28 (2H, sextet), 1.44-1.62 (4H, m), 1.73-1.82 (2H, m), 1.90-2.00 (2H, m), 2.21-2.28 (2H, m), 2.26 (3H, s), 2.50-2.61 (1H, m), 2.89 (2H, t), 3.10-3.20 (2H, m), 3.34-3.41 (2H, m), 4.40-4.49 (2H, m), 4.58 (2H, s), 4.62 (2H, s), 7.09-7.19 (4H, m), 8.60 (1H, s), 11.49 (1H, s).

MSm/z: 580 (M+1).

GTPγS(IC₅₀ μM): 0.0086

### Example 20

### 1-{3-Cyano-6-[(2-oxopyrrolidin-1-yl)methyl]-5-pentanoylpyridin-2-yl}-N-[(2,4-difluorobenzyl)sulfonyl]piperidine-4-carboxamide

Prepared according to Example 1(i) from 1-{3-cyano-6-[(2-oxopyrrolidin-1-yl)methyl]-5-pentanoylpyridin-2-yl}piperidine-4-carboxylic acid (Example 17(b)) (203 mg, 0.49 mmol) by using 1-(2,4-difluorophenyl)methanesulfonamide in place of 1-phenylmethanesulfonamide to give 1-{3-cyano-6-[(2-oxopyrrolidin-1-yl)methyl]-5-pentanoylpyridin-2-yl}-N-[(2,4-difluorobenzyl)sulfonyl]piperidine-4-carboxamide. Purification Method A was used (See General Experimental Procedure). Yield: 125 mg (42%).

¹H-NMR (400 MHz, DMSO-d₆) δ 0.85 (3H, t), 1.28 (2H, sextet), 1.45-1.61 (4H, m), 1.77-1.85 (2H, m), 1.90-1.99 (2H, m), 2.20-2.26 (2H, m), 2.53-2.63 (1H, m), 2.89 (2H, t), 3.11-3.20 (2H, m), 3.34-3.40 (2H, m), 4.41-4.49 (2H, m), 4.62 (2H, s), 4.68 (2H, s), 7.09-7.18 (1H, m), 7.25-7.33 (1H, m), 7.36-7.45 (1H, m), 8.60 (1H, s), 11.70 (1H, s).

MSm/z: 602 (M+1).

GTPγS(IC₅₀ μM): 0.013

### Example 21

### 1-{3-Cyano-6-[(2-oxopyrrolidin-1-yl)methyl]-5-pentanoylpyridin-2-yl}-N-{[(4-fluorophenyl)(methyl)amino]sulfonyl}piperidine-4-carboxamide

Prepared according to Example 1(i) from 1-{3-cyano-6-[(2-oxopyrrolidin-1-yl)methyl]-5-pentanoylpyridin-2-yl}piperidine-4-carboxylic acid (Example 17(b)) (203 mg, 0.49 mmol) by using N-(4-fluorophenyl)-N-methylsulfamide in place of 1-phenylmethanesulfonamide to give 1-{3-cyano-6-[(2-oxopyrrolidin-1-yl)methyl]-5-pentanoylpyridin-2-yl}-N-{[(4-fluorophenyl)(methyl)amino]sulfonyl}piperidine-4-carboxamide. Purification Method A was used (See General Experimental Procedure). Yield: 120 mg (41%).

¹H-NMR (400 MHz, DMSO-d₆) δ 0.85 (3H, t), 1.28 (2H, sextet), 1.43-1.55 (4H, m), 1.68-1.76 (2H, m), 1.89-1.98 (2H, m), 2.19-2.25 (2H, m), 2.50-2.58 (1H, m), 2.89 (2H, t), 3.08-3.17 (2H, m), 3.26 (3H, s), 3.33-3.40 (2H, m), 4.38-4.46 (2H, m), 4.62 (2H, s), 7.17-7.24 (2H, m), 7.27-7.34 (2H, m), 8.59 (1H, s), 11.60 (1H, s).

MSm/z: 599 (M+1).

GTPγS(IC₅₀ μM): 0.023

### Example 22

### 1-{3-Cyano-6-[(2-oxopyrrolidin-1-yl)methyl]-5-pentanoylpyridin-2-yl}-N-{[methyl(phenyl)amino]sulfonyl}piperidine-4-carboxamide

Prepared according to Example 1(i) from 1-{3-cyano-6-[(2-oxopyrrolidin-1-yl)methyl]-5-pentanoylpyridin-2-yl}piperidine-4-carboxylic acid (Example 17(b)) (203 mg, 0.49 mmol) by using N-methyl-N-phenylsulfamide (Example 13(c)) in place of 1-phenylmethanesulfonamide to give 1-{3-cyano-6-[(2-oxopyrrolidin-1-yl)methyl]-5-pentanoylpyridin-2-yl}-N-{[methyl(phenyl)amino]sulfonyl}piperidine-4-carboxamide. Purification Method A was used (See General Experimental Procedure). Yield: 107 mg (37%).

¹H-NMR (400 MHz, DMSO-d₆) δ 0.85 (3H, t), 1.27 (2H, sextet), 1.41-1.53 (4H, m), 1.65-1.72 (2H, m), 1.89-1.98 (2H, m), 2.18-2.25 (2H, m), 2.50-2.56 (1H, m), 2.89 (2H, t), 3.07-3.17 (2H, m), 3.26 (3H, s), 3.31-3.39 (2H, m), 4.36-4.44 (2H, m), 4.61 (2H, s), 7.23-7.30 (3H, m), 7.33-7.39 (2H, m), 8.60 (1H, s), 11.60 (1H, s).

MSm/z: 581 (M+1).

GTPγS(IC₅₀ μM): 0.017

### Example 23

### 1-{3-Cyano-5-(cyclopropylcarbonyl)-6-[(2-oxopyrrolidin-1-yl)methyl]pyridin-2-yl}-N-{[methyl(phenyl)amino]sulfonyl}piperidine-4-carboxamide

Prepared according to Example 1(i) from 1-{3-cyano-5-(cyclopropylcarbonyl)-6-[(2-oxopyrrolidin-1-yl)methyl]pyridin-2-yl}piperidine-4-carboxylic acid (Example 8(b)) (200 mg, 0.5 mmol) by using N-methyl-N-phenylsulfamide (Example 13(c)) in place of 1-phenylmethanesulfonamide to give 1-{3-cyano-5-(cyclopropylcarbonyl)-6-[(2-oxopyrrolidin-1-yl)methyl]pyridin-2-yl}-N-{[methyl(phenyl)amino]sulfonyl}piperidine-4-carboxamide. Yield: 72 mg (27%).

¹H-NMR (400 MHz, DMSO-d₆) δ 0.94 (4H, d), 1.41-1.55 (2H, m), 1.65-1.74 (2H, m), 1.88-1.97 (2H, m), 2.18-2.26 (2H, m), 2.48-2.58 (1H, m), 2.68-2.77 (1H, m), 3.08-3.19 (2H, m), 3.30 (3H, s), 3.32-3.40 (2H, m), 4.37-4.44 (2H, m), 4.58 (2H, s), 7.23-7.30 (3H, m), 7.33-7.40 (2H, m), 8.74 (1H, s), 11.61 (1H, s).

MSm/z: 565 (M+1).

GTPγS(IC₅₀ μM): 0.042

### Example 24

### 1-{3-Cyano-5-(cyclopropylcarbonyl)-6-[(2-oxopyrrolidin-1-yl)methyl]pyridin-2-yl}-N-[(cyclobutylmethyl)sulfonyl]piperidine-4-carboxamide

Prepared according to Example 1(i) from 1-{3-cyano-5-(cyclopropylcarbonyl)-6-[(2-oxopyrrolidin-1-yl)methyl]pyridin-2-yl}piperidine-4-carboxylic acid (Example 8(b)) (200 mg, 0.5 mmol) by using 1-cyclobutylmethanesulfonamide in place of 1-phenylmethanesulfonamide to give 1-{3-Cyano-5-(cyclopropylcarbonyl)-6-[(2-oxopyrrolidin-1-yl)methyl]pyridin-2-yl}-N-[(cyclobutylmethyl)sulfonyl]piperidine-4-carboxamide. Purification Method A was used (See General Experimental Procedure). Yield: 64 mg (24%).

¹H-NMR (400 MHz, DMSO-d₆) δ 0.95 (4H, d), 1.47-1.62 (2H, m), 1.68-2.07 (10H, m), 2.18-2.25 (2H, m), 2.56-2.63 (2H, m), 2.68-2.77 (1H, m), 3.10-3.22 (2H, m), 3.33-3.38 (2H, m), 3.39-3.44 (2H, m), 4.40-4.49 (2H, m), 4.59 (2H, s), 8.74 (1H, s), 11.62 (1H, s).

MSm/z: 528 (M+1).

GTPγS(IC₅₀ μM): 0.428

### Example 25

### 1-{3-Cyano-5-(cyclopropylcarbonyl)-6-[(2-oxopyrrolidin-1-yl)methyl]pyridin-2-yl}-N-{[(4-fluorophenyl)(methyl)amino]sulfonyl}piperidine-4-carboxamide

Prepared according to Example 1(i) from 1-{3-cyano-5-(cyclopropylcarbonyl)-6-[(2-oxopyrrolidin-1-yl)methyl]pyridin-2-yl}piperidine-4-carboxylic acid (Example 8(b)) (200 mg, 0.5 mmol) by using N-(4-fluorophenyl)-N-methylsulfamide in place of 1-phenylmethanesulfonamide to give 1-{3-Cyano-5-(cyclopropylcarbonyl)-6-[(2-oxopyrrolidin-1-yl)methyl]pyridin-2-yl}-N-{[(4-fluorophenyl)(methyl)amino]sulfonyl}piperidine-4-carboxamide. Purification Method A was used (See General Experimental Procedure). Yield: 89 mg (30%).

¹H-NMR (400 MHz, DMSO-d₆) δ 0.95 (4H, d), 1.44-1.59 (2H, m), 1.68-1.80 (2H, m), 1.87-1.98 (2H, m), 2.18-2.26 (2H, m), 2.50-2.59 (1H, m), 2.70-2.77 (1H, m), 3.06-3.19 (2H, m), 3.27 (3H, s), 3.32-3.39 (2H, m), 4.40-4.48 (2H, m), 4.60 (2H, s), 7.19-7.25 (2H, m), 7.27-7.35 (2H, m), 8.75 (1H, s), 11.61 (1H, s).

MSm/z: 583 (M+1).

GTPγS(IC₅₀ μM): 0.026

### Example 26

### N-(benzylsulfonyl)-1-{3-cyano-6-[(2-oxopyrrolidin-1-yl)methyl]-5-pent-4-enoylpyridin-2-yl}piperidine-4-carboxamide

### (a) tert-butyl 1-{3-cyano-6-[(2-oxopyrrolidin-1-yl)methyl]-5-pent-4-enoylpyridin-2-yl}piperidine-4-carboxylate

Prepared according to Example 1(g) from tert-Butyl 1-{5-(chlorocarbonyl)-3-cyano-6-[(2-oxopyrrolidin-1-yl)methyl]pyridin-2-yl}piperidine-4-carboxylate (Example 1(e)) (5 g, 11.7 mmol) by using cyclopropylmethyl magnesium bromide in place of methylmagnesium chloride to give tert-butyl 1-{3-cyano-6-[(2-oxopyrrolidin-1-yl)methyl]-5-pent-4-enoylpyridin-2-yl}piperidine-4-carboxylate. Yield: 1.1 g (20%).

¹H-NMR (400 MHz, CDCl₃) δ 1.43 (9H, s), 1.73-1.81 (2H, m), 1.93-2.09 (4H, m), 2.20-2.25 (1H, m), 2.37-2.44 (3H, m), 2.50-2.55 (1H, m), 2.87-2.90 (2H, m), 3.26-3.35 (2H, m), 3.44-3.47 (2H, m), 4.44-4.50 (2H, m), 4.82 (2H, s), 4.97-5.08 (2H, m), 5.79-5.88 (1H, m), 8.18 (1H, s).

MSm/z: 467 (M+1).

### (b) 1-{3-cyano-6-[(2-oxopyrrolidin-1-yl)methyl]-5-pent-4-enoylpyridin-2-yl}piperidine-4-carboxylic acid

Prepared according to Example 1(h) from tert-butyl 1-{3-cyano-6-[(2-oxopyrrolidin-1-yl)methyl]-5-pent-4-enoylpyridin-2-yl}piperidine-4-carboxylate to give 1-{3-cyano-6-[(2-oxopyrrolidin-1-yl)methyl]-5-pent-4-enoylpyridin-2-yl}piperidine-4-carboxylic acid. Yield: 0.96 g (100%).

MSm/z: 411 (M+1).

### (c) N-(benzylsulfonyl)-1-{3-cyano-6-[(2-oxopyrrolidin-1-yl)methyl]-5-pent-4-enoylpyridin-2-yl}piperidine-4-carboxamide

Prepared according to Example 1(i) from 1-{3-cyano-6-[(2-oxopyrrolidin-1-yl)methyl]-5-pent-4-enoylpyridin-2-yl}piperidine-4-carboxylic acid (240 mg, 0.59 mmol) to give N-(benzylsulfonyl)-1-{3-cyano-6-[(2-oxopyrrolidin-1-yl)methyl]-5-pent-4-enoylpyridin-2-yl}piperidine-4-carboxamide. Purification Method A was used (See General Experimental Procedure). Yield: 113 mg (34%).

¹H-NMR (400 MHz, DMSO-d₆) δ 1.48-1.61 (2H, m), 1.73-1.81 (2H, m), 1.90-1.99 (2H, m), 2.21-2.30 (4H, m), 2.49-2.60 (1H, m), 2.98-3.04 (2H, m), 3.11-3.19 (2H, m), 3.33-3.41 (2H, m), 4.41-4.48 (2H, m), 4.62 (2H, s), 4.64 (2H, s), 4.93 (1H, d), 5.03 (1H, d), 5.76-5.87 (1H, m), 7.18-7.27 (2H, m), 7.30-7.39 (3H, m), 8.62 (1H, s), 11.55 (1H, s).

MSm/z: 564 (M+1).

GTPγS(IC₅₀ μM): 0.017

### Example 27

### 1-{3-cyano-6-[(2-oxopyrrolidin-1-yl)methyl]-5-pent-4-enoylpyridin-2-yl}-N-[(cyclopentylmethyl)sulfonyl]piperidine-4-carboxamide

Prepared according to Example 1(i) from 1-{3-cyano-6-[(2-oxopyrrolidin-1-yl)methyl]-5-pent-4-enoylpyridin-2-yl}piperidine-4-carboxylic acid (Example 26(b)) (240 mg, 0.59 mmol) by using 1-cyclopentylmethanesulfonamide in place of 1-phenylmethanesulfonamide to give 1-{3-cyano-6-[(2-oxopyrrolidin-1-yl)methyl]-5-pent-4-enoylpyridin-2-yl}-N-[(cyclopentylmethyl)sulfonyl]piperidine-4-carboxamide. Purification Method A was used (See General Experimental Procedure). Yield: 63.4 mg (20%).

¹H-NMR (400 MHz, DMSO-d₆) δ 1.12-1.26 (2H, m), 1.41-1.60 (7H, m), 1.75-1.88 (5H, m), 1.88-1.98 (2H, m), 2.05-2.16 (1H, m), 2.19-2.30 (4H, m), 2.58-2.68 (1H, m), 2.96-3.03 (2H, m), 3.12-3.22 (2H, m), 3.31-3.38 (2H, m), 4.40-4.48 (2H, m), 4.61 (2H, s), 4.92 (1H, d), 5.03 (1H, d), 5.74-5.87 (1H, m), 8.62 (1H, s), 11.66 (1H, s).

MSm/z: 556 (M+1).

GTPγS(IC₅₀ μM): 0.02

### Example 28

### 1-{3-cyano-6-[(2-oxopyrrolidin-1-yl)methyl]-5-pent-4-enoylpyridin-2-yl}-N-{[(4-fluorophenyl)(methyl)amino]sulfonyl}piperidine-4-carboxamide

Prepared according to Example 1(i) from 1-{3-cyano-6-[(2-oxopyrrolidin-1-yl)methyl]-5-pent-4-enoylpyridin-2-yl}piperidine-4-carboxylic acid (Example 26(b)) (240 mg, 0.59 mmol) by using N-(4-fluorophenyl)-N-methylsulfamide in place of 1-phenylmethanesulfonamide to give 1-{3-cyano-6-[(2-oxopyrrolidin-1-yl)methyl]-5-pent-4-enoylpyridin-2-yl}-N-{[(4-fluorophenyl)(methyl)amino]sulfonyl}piperidine-4-carboxamide. Purification Method A was used (See General Experimental Procedure). Yield: 121 mg (34%).

¹H-NMR (400 MHz, DMSO-d₆) δ 1.43-1.55 (2H, m), 1.68-1.76 (2H, m), 1.88-1.98 (2H, m), 2.19-2.30 (4H, m), 2.49-2.59 (1H, m), 2.98-3.04 (2H, m), 3.08-3.18 (2H, m), 3.27 (3H, s), 3.33-3.39 (2H, m), 4.39-4.46 (2H, m), 4.62 (2H, s), 4.93 (1H, d), 5.03 (1H, d), 5.75-5.87 (1H, m), 7.17-7.24 (2H, m), 7.28-7.34 (2H, m), 8.62 (1H, s), 11.61 (1H, s).

MSm/z: 597 (M+1).

GTPγS(IC₅₀ μM): 0.022

### Example 29

### 1-{3-cyano-6-[(2-oxopyrrolidin-1-yl)methyl]-5-pent-4-enoylpyridin-2-yl}-N-{[methyl(phenyl)amino]sulfonyl}piperidine-4-carboxamide

Prepared according to Example 1(i) from 1-{3-cyano-6-[(2-oxopyrrolidin-1-yl)methyl]-5-pent-4-enoylpyridin-2-yl}piperidine-4-carboxylic acid (Example 26(b)) (240 mg, 0.59 mmol) by using N-methyl-N-phenylsulfamide (Example 13(c)) in place of 1-phenylmethanesulfonamide to give 1-{3-cyano-6-[(2-oxopyrrolidin-1-yl)methyl]-5-pent-4-enoylpyridin-2-yl}-N-{[methyl(phenyl)amino]sulfonyl}piperidine-4-carboxamide. Purification Method A was used (See General Experimental Procedure). Yield: 114 mg (33%).

¹H-NMR (400 MHz, DMSO-d₆) δ 1.47 (2H, m), 1.69 (2H, m), 1.93 (2H, m), 2.25 (4H, m), 2.49-2.57 (1H, m), 2.97-3.03 (2H, m), 3.07-3.17 (2H, m), 3.29 (3H, s), 4.36-4.44 (2H, m), 4.61 (2H, s), 4.93 (1H, d), 5.02 (1H, d), 5.75-5.86 (1H, m), 7.23-7.39 (5H, m), 8.61 (1H, s), 11.61 (1H, s). Note: The signal from two protones overlapps with the watersignal in the solvent.

MSm/z: 579 (M+1).

GTPγS(IC₅₀ μM): 0.013

### Example 30

### N-(Benzylsulfonyl)-1-{5-butyryl-3-chloro-6-[(2-oxopyrrolidin-1-yl)methyl]pyridin-2-yl}piperidine-4-carboxamide

### (a) Methyl 2-methyl-6-oxo-1,6-dihydropyridine-3-carboxylate

Methyl 3-aminocrotonate (57.6 g, 0.49 mol) and methyl propiolate (50.5 g, 0.6 mol) were dissolved in MeOH, the reaction mixture was refluxed for 18 h. The reaction was cooled to initiate precipitation, the solution was filtered and the solid material was washed with IPA and dried under reduced pressure at 40° C. to give methyl 2-methyl-6-oxo-1,6-dihydropyridine-3-carboxylate. Yield: 11.7 g (14%).

¹H-NMR (400 MHz, DMSO-d₆) δ 2.52 (3H, s), 3.74 (3H, s), 6.20 (1H, d), 7.81 (1H, d), 12.05 (1H, broad s).

MSm/z: 182 (M−1).

### (b) Methyl 5-chloro-2-methyl-6-oxo-1,6-dihydropyridine-3-carboxylate

Methyl 2-methyl-6-oxo-1,6-dihydropyridine-3-carboxylate (28 g, 0.17 mol) was dissolved in DMF (450 mL), N-chlorosuccinimide (24 g, 0.18 mol) was added and the reaction mixture was stirred for 2 h at 100° C. and 2 h at rt. Water (500 mL) was added and the precipitation was filtered, washed with IPA and diethyl ether and dried to give methyl 5-chloro-2-methyl-6-oxo-1,6-dihydropyridine-3-carboxylate as a white solid. Yield: 17.3 g (51%)

¹H-NMR (400 MHz, DMSO-d₆) δ 2.53 (3H, s), 3.76 (3H, s), 8.01 (1H, s), 12.65-12.59 (1H, m).

### (c) Methyl 5,6-dichloro-2-methylnicotinate

Methyl 5-chloro-2-methyl-6-oxo-1,6-dihydropyridine-3-carboxylate (17.3 g, 86 mmol) was added to phosphorus oxychloride (72 mL, 0.77 mol), the reaction mixture was stirred at 110° C. for 6 h. The solvent was concentrated in vacuo, sat. NaHCO₃ (200 mL) and DCM (200 mL) were added, the organic phase was separated and eluated through a silica plug. The solvent was concentrated in vacuo to give methyl 5,6-dichloro-2-methylnicotinate as yellow oil. Yield: 16.5 g (87%)

¹H-NMR (400 MHz, DMSO-d₆) δ 2.68 (3H, s), 3.87 (3H, s), 8.40 (1H, s).

### (d) Methyl 2-(bromomethyl)-5,6-dichloronicotinate

Methyl 5,6-dichloro-2-methylnicotinate (21 g, 95 mmol) was dissolved in CCl₄, NBS (18.7 g, 105 mmol) and benzoylperoxide (23 g, 95 mmol) were added. The reaction mixture was stirred at 95° C. for 3 h, water was added and the solution was filtered. The organic phase was separated and concentrated in vacuo, the residueal oil (31.7 g) was purified by preparative HPLC (Kromasil C8) to give methyl 2-(bromomethyl)-5,6-dichloronicotinate. Yield: 12.4 g (42%).

¹H-NMR (400 MHz, DMSO-d₆) δ 3.91 (3H, s), 4.93 (2H, s), 8.51 (1H, s).

MSm/z: 299 (M+1).

### (e) Methyl 5,6-dichloro-2-[(2-oxopyrrolidin-1-yl)methyl]nicotinate

Methyl 2-(bromomethyl)-5,6-dichloronicotinate (10.3 g, 33 mmol) and 5-methoxy-3,4-dihydro-2H-pyrrole (9.8 g, 99 mmol) were dissolved in NMP, the reaction mixture was stirred at 100° C. for 2 h. Water was added and the solid material was filtered of and washed with water to give methyl 5,6-dichloro-2-[(2-oxopyrrolidin-1-yl)methyl]nicotinate. Yield: 8.11 g (81%).

¹H-NMR (400 MHz, DMSO-d₆) δ 1.97 (2H, m), 2.27 (2H, t), 3.37 (2H, t), 3.88 (3H, s), 4.77 (2H, s), 8.45 (1H, s).

MSm/z: 303 (M+1).

### (f) Methyl 6-[4-(tert-butoxycarbonyl)piperidin-1-yl]-5-chloro-2-[(2-oxopyrrolidin-1-yl)methyl]nicotinate

Methyl 5,6-dichloro-2-[(2-oxopyrrolidin-1-yl)methyl]nicotinate (8.1 g, 26 mmol) and tert-butyl piperidine-4-carboxylate (7.4 g, 40 mmol) were added to a solution of DIPEA (9 mL, 51 mmol) in MeOH (45 mL), the reaction mixture was heated in the micro-wave at 100° C. for 20 min. The solution was concentrated in vacuo, extracted with DCM/0.5M KHSO₄ and concentrated in vacuo to give methyl 6-[4-(tert-butoxycarbonyl)piperidin-1-yl]-5-chloro-2-[(2-oxopyrrolidin-1-yl)methyl]nicotinate as a light brown oil. The crude product was used in the next step without further purification. Yield: 16.9 g (99%).

MSm/z: 452 (M+1).

### (g) 6-[4-(tert-Butoxycarbonyl)piperidin-1-yl]-5-chloro-2-[(2-oxopyrrolidin-1-yl)methyl]nicotinic acid

Methyl 6-[4-(tert-butoxycarbonyl)piperidin-1-yl]-5-chloro-2-[(2-oxopyrrolidin-1-yl)methyl]nicotinate (16.9 g, 25.4 mmol) was dissolved in MeOH (50 mL), 2M NaOH was added and the reaction mixture was stirred at 60° C. for 50 min. The MeOH was concentrated in vacuo and reaction mixture was acidified with acetic acid. The precipitating was filtered, washed and dried to give 6-[4-(tert-butoxycarbonyl)piperidin-1-yl]-5-chloro-2-[(2-oxopyrrolidin-1-yl)methyl]nicotinic acid. Yield: 25.5 g (100%).

¹H-NMR (600 MHz, CDCl₃) δ 1.44 (9H, s), 1.76-1.84 (2H, m), 1.92-1.97 (2H, m), 2.07-2.13 (2H, m), 2.41-2.46 (1H, m), 2.48-2.53 (2H, m), 2.97-3.03 (2H, m), 3.58-3.61 (2H, m), 4.01-4.06 (2H, m), 4.86 (2H, s), 8.04 (1H, s).

MSm/z: 438 (M+1), 436 (M−1).

### (h) tert-Butyl 1-{3-chloro-5-(fluorocarbonyl)-6-[(2-oxopyrrolidin-1-yl)methyl]pyridin-2-yl}piperidine-4-carboxylate

6-[4-(tert-Butoxycarbonyl)piperidin-1-yl]-5-chloro-2-[(2-oxopyrrolidin-1-yl)methyl]nicotinic acid (11.2 g, 25.5 mmol) was added to a solution of cyanuric fluoride (3.78 g, 28 mmol) and pyridine (2.5 mL, 30.6 mmol) in DCM (100 mL) at 0° C., the temperature was slowly reached r.t. The solution was filtered, water and DCM were added and the organic phase was separated and concentrated in vacuo. The crude product was used in the next step without further purification. Yield: 9.85 g (72%).

### (i) tert-Butyl 1-{5-butyryl-3-chloro-6-[(2-oxopyrrolidin-1-yl)methyl]pyridin-2-yl}piperidine-4-carboxylate

Prepared according to Example 1(g) from tert-butyl 1-{3-chloro-5-(fluorocarbonyl)-6-[(2-oxopyrrolidin-1-yl)methyl]pyridin-2-yl}piperidine-4-carboxylate (1.02 g, 1.9 mmol) by using propyl magnesium bromide in place of methylmagnesium chloride to give tert-butyl 1-{5-butyryl-3-chloro-6-[(2-oxopyrrolidin-1-yl)methyl]pyridin-2-yl}piperidine-4-carboxylate. Yield: 0.56 g (63%).

MSm/z: 464 (M+1)

### (j) 1-{5-Butyryl-3-chloro-6-[(2-oxopyrrolidin-1-yl)methyl]pyridin-2-yl}piperidine-4-carboxylic acid

Prepared according to Example 1(h) from tert-butyl 1-{5-butyryl-3-chloro-6-[(2-oxopyrrolidin-1-yl)methyl]pyridin-2-yl}piperidine-4-carboxylate (0.89 g, 1.91 mmol) to give 1-{5-butyryl-3-chloro-6-[(2-oxopyrrolidin-1-yl)methyl]pyridin-2-yl}piperidine-4-carboxylic acid. Yield: 0.78 g (100%).

MSm/z: 408 (M+1), MSm/z: 406 (M−1)

### (k) N-(Benzylsulfonyl)-1-{5-butyryl-3-chloro-6-[(2-oxopyrrolidin-1-yl)methyl]pyridin-2-yl}piperidine-4-carboxamide

To a solution of 1-{5-butyryl-3-chloro-6-[(2-oxopyrrolidin-1-yl)methyl]pyridin-2-yl}piperidine-4-carboxylic acid (Example 103j) (0.22 g, 0.54 mmol) in DCM (6 mL) were TBTU (0.23 g, 0.72 mmol) and DIPEA (0.20 g, 1.56 mmol) added. The reaction was stirred for 30 min at rt, 1-phenylmethanesulfonamide (0.11 g, 0.66 mmol) was added and the reaction was stirred for 45 min at r.t. DCM and water were added, the organic phase was separated and concentrated in vacuo. The residue was purified by HPLC (Kromasil C8 using a gradient of 20% to 55% of MeCN with a second acidic eluent (H₂O/MeCN/AcOH, 95/5/0.2)) to give N-(benzylsulfonyl)-1-{5-butyryl-3-chloro-6-[(2-oxopyrrolidin-1-yl)methyl]pyridin-2-yl}piperidine-4-carboxamide. Yield: 0.118 g (39%)

¹H-NMR (400 MHz, CD₃OD) δ 0.99 (3H, t), 1.65-1.75 (2H, m), 1.78-1.85 (4H, m), 2.13 (2H, quintett), 2.38-2.50 (3H, m), 2.87-3.00 (4H, m), 3.56 (2H, t), 4.12-4.19 (2H, m), 4.66 (2H, s), 4.76 (2H, s), 7.35-7.40 (5H, m), 8.20 (1H, s).

MSm/z: 561 (M+1), 559 (M−1).

GTPγS(IC₅₀ μM): 0.062

### Example 31

### 1-{5-Butyryl-3-chloro-6-[(2-oxopyrrolidin-1-yl)methyl]pyridin-2-yl}-N-{[(4-fluorophenyl)(methyl)amino]sulfonyl}piperidine-4-carboxamide

Prepared according to Example 1(i) from 1-{5-butyryl-3-chloro-6-[(2-oxopyrrolidin-1-yl)methyl]pyridin-2-yl}piperidine-4-carboxylic acid (350 mg, 0.86 mmol) by using N-(4-fluorophenyl)-N-methylsulfamide in place of 1-phenylmethanesulfonamide to give 1-{5-Butyryl-3-chloro-6-[(2-oxopyrrolidin-1-yl)methyl]pyridin-2-yl}-N-{[(4-fluorophenyl)(methyl)amino]sulfonyl}piperidine-4-carboxamide. Yield: 270 mg (53%).

¹H-NMR (400 MHz, DMSO-d₆) δ 0.89 (3H, t), 1.51-1.62 (4H, m), 1.69-1.75 (2H, m), 1.91-2.00 (2H, m), 2.22-2.27 (2H, m), 2.40-2.47 (1H, m), 2.85-2.93 (4H, m), 3.29 (3H, s), 3.36-3.43 (2H, m), 3.99-4.07 (2H, m), 4.60 (2H, s), 7.20-7.27 (2H, m), 7.31-7.37 (2H, m), 8.25 (1H, s), 11.35-11.82 (1H, m).

MSm/z: 594 (M+1), 592 (M−1).

GTPγS(IC₅₀ μM): 0.109

### Example 32

### 1-{5-Butyryl-3-chloro-6-[(2-oxopyrrolidin-1-yl)methyl]pyridin-2-yl}-N-[(4-methylbenzyl)sulfonyl]piperidine-4-carboxamide

Prepared according to Example 1(i) from 1-{5-butyryl-3-chloro-6-[(2-oxopyrrolidin-1-yl)methyl]pyridin-2-yl}piperidine-4-carboxylic acid (Example 30 (j))(67 mg, 0.16 mmol) by using 1-(4-methylphenyl)methanesulfonamide in place of 1-phenylmethanesulfonamide to give 1-{5-butyryl-3-chloro-6-[(2-oxopyrrolidin-1-yl)methyl]pyridin-2-yl}-N-[(4-methylbenzyl)sulfonyl]piperidine-4-carboxamide. Yield: 62 mg (66%).

¹H-NMR (600 MHz, CDCl₃) δ 0.98 (3H, t), 1.66-1.72 (3H, m), 1.77-1.83 (3H, m), 2.09-2.16 (2H, m), 2.33 (3H, s), 2.39-2.44 (1H, m), 2.46 (2H, t), 2.90 (2H, t), 2.93-2.99 (2H, m), 3.55 (2H, t), 4.13-4.17 (2H, m), 4.61 (2H, s), 4.75 (2H, s), 7.18-7.25 (4H, m), 8.19 (1H, s).

MSm/z: 575 (M+1), 573 (M−1).

GTPγS(IC₅₀ μM): 0.055

### Example 33

### 1-{5-Butyryl-3-cyano-6-[(2-oxopyrrolidin-1-yl)methyl]pyridin-2-yl}-N-{[(4-fluorophenyl)(methyl)amino]sulfonyl}piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (Example 2(b)) (254 mg, 0.64 mmol) by using N-(4-fluorophenyl)-N-methylsulfamide in place of 1-phenylmethanesulfonamide to give 1-{5-butyryl-3-cyano-6-[(2-oxopyrrolidin-1-yl)methyl]pyridin-2-yl}-N-{[(4-fluorophenyl)(methyl)amino]sulfonyl}piperidine-4-carboxamide. Yield: 237 mg (64%).

¹H-NMR (400 MHz, CDCl₃) δ 0.88-1.06 (3H, m), 1.59-1.89 (6H, m), 1.97-2.14 (2H, m), 2.27-2.39 (2H, m), 2.46-2.57 (1H, m), 2.73-2.84 (2H, m), 3.06-3.22 (2H, m), 3.32-3.52 (5H, m), 4.38-4.57 (2H, m), 4.80 (2H, s), 6.97-7.12 (2H, m), 7.19-7.39 (2H, m), 8.21 (1H, s), 9.94 (1H, s).

MSm/z: 585 (M+1), 583 (M−1)

GTPγS(IC₅₀ μM): 0.011

### Example 34

### 1-{5-Butyryl-3-cyano-6-[(2-oxopyrrolidin-1-yl)methyl]pyridin-2-yl}-N-{[methyl(phenyl)amino]sulfonyl}piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (Example 2(b)) (254 mg, 0.64 mmol) by using N-methyl-N-phenylsulfamide (Example 13(c)) in place of 1-phenylmethanesulfonamide to give 1-{5-butyryl-3-cyano-6-[(2-oxopyrrolidin-1-yl)methyl]pyridin-2-yl}-N-{[methyl(phenyl)amino]sulfonyl}piperidine-4-carboxamide. Yield: 224 mg (62%).

¹H-NMR (400 MHz, DMSO-d₆) δ 0.90 (3H, t), 1.44-1.62 (4H, m), 1.68-1.76 (2H, m), 1.92-2.02 (2H, m), 2.22-2.29 (2H, m), 2.50-2.58 (1H, m), 2.90 (2H, t), 3.11-3.22 (2H, m), 3.32 (3H, s), 3.36-3.46 (2H, m), 4.38-4.46 (2H, m), 4.66 (2H, s), 7.23-7.42 (5H, m), 8.61 (1H, s).

MSm/z: 567 (M+1).

GTPγS(IC₅₀ μM): 0.012

### Example 35

### 1-{5-Butyryl-3-cyano-6-[(2-oxopyrrolidin-1-yl)methyl]pyridin-2-yl}-N-[(4-methoxybenzyl)sulfonyl]piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (Example 2(b)) (255 mg, 0.64 mmol) by using 1-(4-methoxyphenyl)methanesulfonamide in place of 1-phenylmethanesulfonamide to give 1-{5-butyryl-3-cyano-6-[(2-oxopyrrolidin-1-yl)methyl]pyridin-2-yl}-N-[(4-methoxybenzyl)sulfonyl]piperidine-4-carboxamide. Yield: 248 mg (67%).

¹H-NMR (400 MHz, DMSO-d₆) δ 0.90 (3H, t), 1.52-1.66 (4H, m), 1.78-1.86 (2H, m), 1.94-2.04 (2H, m), 2.24-2.31 (2H, m), 2.54-2.64 (1H, m), 2.91 (2H, t), 3.14-3.25 (2H, m), 3.37-3.45 (2H, m), 3.74 (3H, s), 4.45-4.53 (2H, m), 4.58 (2H, s), 4.66 (2H, s), 6.94 (2H, d), 7.19 (2H, d), 8.62 (1H, s).

MSm/z: 582 (M+1).

GTPγS(IC₅₀ μM): 0.0097

### Example 36

### 1-{5-Butyryl-3-cyano-6-[(2-oxopyrrolidin-1-yl)methyl]pyridin-2-yl}-N-[(cyclohexylmethyl)sulfonyl]piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (Example 2(b)) (255 mg, 0.64 mmol) by using 1-cyclohexylmethanesulfonamide in place of 1-phenylmethanesulfonamide to give 1-{5-butyryl-3-cyano-6-[(2-oxopyrrolidin-1-yl)methyl]pyridin-2-yl}-N-[(cyclohexylmethyl)sulfonyl]piperidine-4-carboxamide. Yield: 248 mg (67%).

MSm/z: 558 (M+1).

GTPγS(IC₅₀ μM): 0.014

### Example 37

### 1-{5-Butyryl-3-cyano-6-[(2-oxopyrrolidin-1-yl)methyl]pyridin-2-yl}-N-{[(4-fluorophenyl)amino]sulfonyl}piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (Example 2(b)) (100 mg, 0.25 mmol) by using N-(4-fluorophenyl)sulfamide in place of 1-phenylmethanesulfonamide to give 1-{5-butyryl-3-cyano-6-[(2-oxopyrrolidin-1-yl)methyl]pyridin-2-yl}-N-{[(4-fluorophenyl)amino]sulfonyl}piperidine-4-carboxamide. Yield: 67.7 mg (47%).

¹H-NMR (400 MHz, DMSO-d₆) δ 0.90 (3H, t), 1.34-1.46 (2H, m), 1.50-1.67 (4H, m), 1.89-1.98 (2H, m), 2.19-2.25 (2H, m), 2.88-2.93 (2H, m), 3.12-3.21 (2H, m), 3.35-3.41 (2H, m), 4.30-4.38 (2H, m), 4.64 (2H, s), 7.10-7.17 (4H, m), 8.60 (1H, s). MSm/z: 571 (M+1). One signal (1H) is overlapping with the solvent signal.

GTPγS(IC₅₀ μM): 0.019

### Example 38

### N-(Anilinosulfonyl)-1-{5-butyryl-3-cyano-6-[(2-oxopyrrolidin-1-yl)methyl]pyridin-2-yl}piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (Example 2(b)) (100 mg, 0.25 mmol) by using N-phenylsulfamide in place of 1-phenylmethanesulfonamide to give N-(anilinosulfonyl)-1-{5-butyryl-3-cyano-6-[(2-oxopyrrolidin-1-yl)methyl]pyridin-2-yl}piperidine-4-carboxamide. Yield: 65.4 mg (47%).

¹H-NMR (400 MHz, DMSO-d₆) δ 0.89 (3H, t), 1.33-1.45 (2H, m), 1.50-1.69 (4H, m), 1.90-1.99 (2H, m), 2.19-2.26 (2H, m), 2.37-2.45 (1H, m), 2.86-2.94 (2H, m), 3.11-3.25 (2H, m), 4.24-4.32 (2H, m), 4.64 (2H, s), 6.97-7.03 (1H, m), 7.07-7.13 (2H, m), 7.19-7.28 (2H, m), 8.58 (1H, s). The signal from two of the protons are overlapping with the water signal in the solvent.

MSm/z: 553 (M+1).

GTPγS(IC₅₀ μM): 0.033

### Example 39

### 1-{5-Butyryl-3-chloro-6-[(2-oxopyrrolidin-1-yl)methyl]pyridin-2-yl}-N-[(cyclopentylmethyl)sulfonyl]piperidine-4-carboxamide

Prepared according to Example 1(i) from 1-{5-butyryl-3-cyano-6-[(2-oxopyrrolidin-1-yl)methyl]pyridin-2-yl}piperidine-4-carboxylic acid (Example 2(b)) (36 mg, 0.088 mmol) by using 1-cyclopentylmethanesulfonamide in place of 1-phenylmethanesulfonamide to give 1-{5-butyryl-3-chloro-6-[(2-oxopyrrolidin-1-yl)methyl]pyridin-2-yl}-N-[(cyclopentylmethyl)sulfonyl]piperidine-4-carboxamide. Yield: 3.1 mg (6%).

¹H-NMR (500 MHz, DMSO-d₆) δ 0.91 (3H, t), 1.19-1.28 (3H, m), 1.46-1.66 (8H, m), 1.81-1.88 (4H, m), 1.95-2.02 (2H, m), 2.11-2.18 (1H, m), 2.24-2.29 (2H, m), 2.54 (2H, s), 2.89-2.98 (4H, m), 3.41-3.46 (3H, m), 4.03-4.09 (2H, m), 4.61 (2H, s), 8.27 (1H, s).

GTPγS(IC₅₀ μM): 0.72

### Example 40

### 1-{3-cyano-6-[(2-oxopyrrolidin-1-yl)methyl]-5-pent-4-enoylpyridin-2-yl}-N-[(4-methylbenzyl)sulfonyl]piperidine-4-carboxamide

Prepared according to Example 1(h) from 1-{3-cyano-6-[(2-oxopyrrolidin-1-yl)methyl]-5-pent-4-enoylpyridin-2-yl}piperidine-4-carboxylic acid (Example 26(b)) (73 mg, 0.18 mmol) to give 1-{3-cyano-6-[(2-oxopyrrolidin-1-yl)methyl]-5-pent-4-enoylpyridin-2-yl}-N-[(4-methylbenzyl)sulfonyl]piperidine-4-carboxamide. Yield: 11 mg (11%).

MSm/z: 578 (M+1).

GTPγS(IC₅₀ μM): 0.0081

### Example 41

### 1-{3-cyano-6-[(2-oxopyrrolidin-1-yl)methyl]-5-pent-4-enoylpyridin-2-yl}-N-[(2,4-difluorobenzyl)sulfonyl]piperidine-4-carboxamide

Prepared according to Example 1(i) from 1-{3-cyano-6-[(2-oxopyrrolidin-1-yl)methyl]-5-pent-4-enoylpyridin-2-yl}piperidine-4-carboxylic acid (Example 26(b)) (73 mg, 0.18 mmol) to give 1-{3-cyano-6-[(2-oxopyrrolidin-1-yl)methyl]-5-pent-4-enoylpyridin-2-yl}-N-[(2,4-difluorobenzyl)sulfonyl]piperidine-4-carboxamide. Yield: 8 mg (8%).

MSm/z: 600 (M+1).

GTPγS(IC₅₀ μM): 0.009

### Example 42

### 1-{3-Cyano-6-[(2-oxopyrrolidin-1-yl)methyl]-5-(4,4,4-trifluorobutanoyl)pyridin-2-yl}-N-[(2,4-difluorobenzyl)sulfonyl]piperidine-4-carboxamide

### (a) tert-Butyl 1-{3-cyano-5-(fluorocarbonyl)-6-[(2-oxopyrrolidin-1-yl)methyl]pyridin-2-yl}piperidine-4-carboxylate

Prepared according to Example 30(h) from 6-[4-(tert-butoxycarbonyl)piperidin-1-yl]-5-cyano-2-[(2-oxopyrrolidin-1-yl)methyl]nicotinic acid (Example 1(d)) (1.0 g, 2.33 mmol) to give tert-butyl 1-{3-cyano-5-(fluorocarbonyl)-6-[(2-oxopyrrolidin-1-yl)methyl]pyridin-2-yl}piperidine-4-carboxylate. Yield: 903 mg (90%).

¹H-NMR (400 MHz, CDCl₃) δ 1.44 (9H, s), 1.74-1.85 (2H, m), 1.98-2.06 (2H, m), 2.06-2.16 (2H, m), 2.45-2.51 (2H, m), 2.53-2.62 (1H, m), 3.39-3.52 (4H, m), 4.49-4.56 (2H, m), 4.85 (2H, s), 8.28 (1H, s).

### (b) tert-Butyl 1-{3-cyano-6-[(2-oxopyrrolidin-1-yl)methyl]-5-(4,4,4-trifluorobutanoyl)pyridin-2-yl}piperidine-4-carboxylate

tert-Butyl 1-{3-cyano-5-(fluorocarbonyl)-6-[(2-oxopyrrolidin-1-yl)methyl]pyridin-2-yl}piperidine-4-carboxylate (100 mg, 0.23 mmol) and ferric acetylacetonate were dissolved in THF (1 mL), the solution was cooled to −78° C. n-Trifluoropropylmagnesium bromide (55 mg, 0.26 mmol) was added and the temperature was increased to room temperature. NH₃Cl (sat.) and EtOAc were added, the organic phase was separated, dried (Na₂SO₄) and concentrated in vacuo. The residue was purified by HPLC, (Kromasil C8, 250×50 Idmm using a gradient of 10% to 90% MeCN with an acidic second eluent (H₂O/MeCN/FA: 95:5:0.2)) to give tert-butyl 1-{3-cyano-6-[(2-oxopyrrolidin-1-yl)methyl]-5-(4,4,4-trifluorobutanoyl)pyridin-2-yl}piperidine-4-carboxylate. Yield: 28 mg (24%).

### (c) 1-{3-cyano-6-[(2-oxopyrrolidin-1-yl)methyl]-5-(4,4,4-trifluorobutanoyl)pyridin-2-yl}piperidine-4-carboxylic acid

Prepared according to Example 1(h) from tert-butyl 1-{3-cyano-6-[(2-oxopyrrolidin-1-yl)methyl]-5-(4,4,4-trifluorobutanoyl)pyridin-2-yl}piperidine-4-carboxylate (928 mg, 1.82 mmol) to give 1-{3-cyano-6-[(2-oxopyrrolidin-1-yl)methyl]-5-(4,4,4-trifluorobutanoyl)pyridin-2-yl}piperidine-4-carboxylic acid. Yield: 115 mg (14%).

### (d) 1-{3-Cyano-6-[(2-oxopyrrolidin-1-yl)methyl]-5-(4,4,4-trifluorobutanoyl)pyridin-2-yl}-N-[(2,4-difluorobenzyl)sulfonyl]piperidine-4-carboxamide

Prepared according to Example 13(d) from 1-{3-cyano-6-[(2-oxopyrrolidin-1-yl)methyl]-5-(4,4,4-trifluorobutanoyl)pyridin-2-yl}piperidine-4-carboxylic acid (30 mg, 0.07 mmol) and 1-(2,4-difluorophenyl)methanesulfonamide (19 mg, 0.09 mmol) to give 1-{3-cyano-6-[(2-oxopyrrolidin-1-yl)methyl]-5-(4,4,4-trifluorobutanoyl)pyridin-2-yl}-N-[(2,4-difluorobenzyl)sulfonyl]piperidine-4-carboxamide. Yield: 16 mg (24%).

¹H-NMR (400 MHz, CDCl₃) δ 1.80-1.89 (2H, m), 1.94-2.01 (2H, m), 2.06-2.15 (2H, m), 2.39-2.45 (2H, m), 2.50-2.62 (3H, m), 3.08-3.12 (2H, m), 3.28-3.37 (2H, m), 3.44-3.51 (2H, m), 4.49-4.63 (3H, m), 4.70 (2H, s), 4.84 (2H, s), 6.86-6.98 (2H, m), 7.35-7.44 (1H, m), 8.36-8.40 (1H, m).

GTPγS(IC₅₀ μM): 0.016

### Example 43

### 1-{3-cyano-6-[(2-oxopyrrolidin-1-yl)methyl]-5-(4,4,4-trifluorobutanoyl)pyridin-2-yl}-N-[(4-methylbenzyl)sulfonyl]piperidine-4-carboxamide

Prepared according to Example 13(d) from 1-{3-cyano-6-[(2-oxopyrrolidin-1-yl)methyl]-5-(4,4,4-trifluorobutanoyl)pyridin-2-yl}piperidine-4-carboxylic acid (Example 42(c)) (30 mg, 0.07 mmol) and 1-(4-methylphenyl)methanesulfonamide (17 mg, 0.09 mmol) to give 1-{3-cyano-6-[(2-oxopyrrolidin-1-yl)methyl]-5-(4,4,4-trifluorobutanoyl)pyridin-2-yl}-N-[(4-methylbenzyl)sulfonyl]piperidine-4-carboxamide. Yield: 13.2 mg (5%).

¹H-NMR (400 MHz, CDCl₃) δ 1.74-1.85 (2H, m), 1.87-1.94 (2H, m), 2.05-2.15 (2H, m), 2.37 (3H, s), 2.39-2.62 (5H, m), 3.07-3.12 (2H, m), 3.25-3.33 (2H, m), 3.45-3.50 (2H, m), 4.47-4.60 (3H, m), 4.62 (2H, s), 4.83 (2H, s), 7.17-7.24 (4H, m), 8.20 (1H, s).

GTPγS(IC₅₀ μM): 0.019

### Example 44

### 1-{3-cyano-6-[(2-oxopyrrolidin-1-yl)methyl]-5-(4,4,4-trifluorobutanoyl)pyridin-2-yl}-N-[(cyclopentylmethyl)sulfonyl]piperidine-4-carboxamide

Prepared according to Example 13(d) from 1-{3-cyano-6-[(2-oxopyrrolidin-1-yl)methyl]-5-(4,4,4-trifluorobutanoyl)pyridin-2-yl}piperidine-4-carboxylic acid (Example 42(c)) (30 mg, 0.07 mmol) and 1-cyclopentylmethanesulfonamide (15 mg, 0.09 mmol) to give 1-{3-cyano-6-[(2-oxopyrrolidin-1-yl)methyl]-5-(4,4,4-trifluorobutanoyl)pyridin-2-yl}-N-[(cyclopentylmethyl)sulfonyl]piperidine-4-carboxamide. Yield: 4 mg (10%).

MSm/z: 598 (M+1), 597 (M−1)

GTPγS(IC₅₀ μM): 0.146

### Example 45

### 1-{3-cyano-6-[(2-oxopyrrolidin-1-yl)methyl]-5-(4,4,4-trifluorobutanoyl)pyridin-2-yl}-N-{[(4-fluorophenyl)(methyl)amino]sulfonyl}piperidine-4-carboxamide

Prepared according to Example 13(d) from 1-{3-cyano-6-[(2-oxopyrrolidin-1-yl)methyl]-5-(4,4,4-trifluorobutanoyl)pyridin-2-yl}piperidine-4-carboxylic acid (Example 42(c)) (30 mg, 0.07 mmol) and N-(4-fluorophenyl)-N-methylsulfamide (19 mg, 0.09 mmol) to give 1-{3-cyano-6-[(2-oxopyrrolidin-1-yl)methyl]-5-(4,4,4-trifluorobutanoyl)pyridin-2-yl}-N-{[(4-fluorophenyl)(methyl)amino]sulfonyl}piperidine-4-carboxamide. Yield: 13 mg (31%).

¹H-NMR (400 MHz, CDCl₃) δ 1.71-1.92 (4H, m), 2.04-2.13 (2H, m), 2.38-2.62 (5H, m), 3.06-3.13 (2H, m), 3.23-3.30 (2H, m), 3.43-3.48 (2H, m), 3.49 (3H, s), 4.53-4.61 (3H, m), 4.82 (2H, s), 7.06-7.12 (2H, m), 7.31-7.38 (2H, m), 8.21 (1H, s).

GTPγS(IC₅₀ μM): 0.05

### Example 46

### 1-{3-cyano-6-[(2-oxopyrrolidin-1-yl)methyl]-5-pentanoylpyridin-2-yl}-N-{[(4-fluorophenyl)amino]sulfonyl}piperidine-4-carboxamide

Prepared according to Example 13(d) from 1-{3-cyano-6-[(2-oxopyrrolidin-1-yl)methyl]-5-pentanoylpyridin-2-yl}piperidine-4-carboxylic acid (Example 17(b)) (125 mg, 0.3 mmol) to give 1-{3-cyano-6-[(2-oxopyrrolidin-1-yl)methyl]-5-pentanoylpyridin-2-yl}-N-{[(4-fluorophenyl)amino]sulfonyl}piperidine-4-carboxamide. Yield: 88 mg (50%).

¹H-NMR (400 MHz, DMSO-d₆) δ 0.88 (3H, t), 1.26-1.43 (4H, m), 1.46-1.55 (2H, m), 1.59-1.66 (2H, m), 1.90-1.97 (2H, m), 2.18-2.24 (2H, m), 2.91 (3H, t), 3.10-3.19 (2H, m), 3.31-3.40 (2H, m), 4.31-4.38 (2H, m), 4.63 (2H, s), 7.09-7.19 (4H, m), 8.62 (1H, s), 10.34 (1H, s), 11.68 (1H, s).

GTPγS(IC₅₀ μM): 0.021

### Example 47

### N-[(4-cyanobenzyl)sulfonyl]-1-{3-cyano-6-[(2-oxopyrrolidin-1-yl)methyl]-5-pentanoylpyridin-2-yl}piperidine-4-carboxamide

Prepared according to Example 13(d) from 1-{3-cyano-6-[(2-oxopyrrolidin-1-yl)methyl]-5-pentanoylpyridin-2-yl}piperidine-4-carboxylic acid (Example 17(b)) (125 mg, 0.3 mmol) to give N-[(4-cyanobenzyl)sulfonyl]-1-{3-cyano-6-[(2-oxopyrrolidin-1-yl)methyl]-5-pentanoylpyridin-2-yl}piperidine-4-carboxamide. Yield: 133 mg (74%).

¹H-NMR (400 MHz, CDCl₃) δ 0.95 (3H, t), 1.34-1.44 (2H, m), 1.62-1.89 (6H, m), 2.02-2.11 (2H, m), 2.29-2.38 (2H, m), 2.51-2.60 (1H, m), 2.80-2.85 (2H, m), 3.13-3.24 (2H, m), 3.38-3.46 (2H, m), 4.41-4.48 (2H, m), 4.68 (2H, s), 4.80 (2H, s), 7.49 (2H, d), 7.69 (2H, d), 8.24 (1H, s), 10.13 (1H, s).

GTPγS(IC₅₀ μM): 0.043

### Example 48

### 1-{3-cyano-6-[(2-oxopyrrolidin-1-yl)methyl]-5-pentanoylpyridin-2-yl}-N-[(cyclohexylmethyl)sulfonyl]piperidine-4-carboxamide

Prepared according to Example 13(d) from 1-{3-cyano-6-[(2-oxopyrrolidin-1-yl)methyl]-5-pentanoylpyridin-2-yl}piperidine-4-carboxylic acid (Example 17(b)) (125 mg, 0.3 mmol) to give 1-{3-cyano-6-[(2-oxopyrrolidin-1-yl)methyl]-5-pentanoylpyridin-2-yl}-N-[(cyclohexylmethyl)sulfonyl]piperidine-4-carboxamide. Yield: 86 mg (50%).

¹H-NMR (400 MHz, CDCl₃) δ 0.93-0.97 (3H, m), 1.05-1.44 (7H, m), 1.62-2.04 (12H, m), 2.06-2.15 (2H, m), 2.48 (2H, t), 2.59-2.68 (1H, m), 2.82 (2H, t), 3.25-3.34 (4H, m), 3.44-3.48 (2H, m), 4.46-4.53 (2H, m), 4.86 (2H, s), 8.23 (1H, s), 9.65 (1H, s).

GTPγS(IC₅₀ μM): 0.023

### Example 49

### 1-{3-cyano-6-[(2-oxopyrrolidin-1-yl)methyl]-5-pentanoylpyridin-2-yl}-N-[(4-isopropylbenzyl)sulfonyl]piperidine-4-carboxamide

Prepared according to Example 13(d) from 1-{3-cyano-6-[(2-oxopyrrolidin-1-yl)methyl]-5-pentanoylpyridin-2-yl}piperidine-4-carboxylic acid (Example 17(b)) (125 mg, 0.3 mmol) to give 1-{3-cyano-6-[(2-oxopyrrolidin-1-yl)methyl]-5-pentanoylpyridin-2-yl}-N-[(4-isopropylbenzyl)sulfonyl]piperidine-4-carboxamide. Yield: 178 mg (75%).

¹H-NMR (400 MHz, CDCl₃) δ 0.95 (3H, t), 1.23 (6H, d), 1.33-1.44 (2H, m), 1.61-1.70 (2H, m), 1.73-1.91 (3H, m), 2.02-2.10 (2H, m), 2.28-2.35 (2H, m), 2.49-2.57 (1H, m), 2.81 (2H, t), 2.87-2.95 (2H, m), 3.18-3.27 (2H, m), 3.44 (2H, t), 4.45-4.52 (2H, m), 4.60 (2H, s), 4.82 (2H, s), 7.20-7.28 (4H, m), 8.22 (1H, s), 9.31-9.41 (1H, m).

GTPγS(IC₅₀ μM): 0.031

### Example 50

### 1-{3-chloro-6-[(2-oxopyrrolidin-1-yl)methyl]-5-pent-4-enoylpyridin-2-yl}-N-{[(4-fluorophenyl)(methyl)amino]sulfonyl}piperidine-4-carboxamide

### (a) tert-Butyl 1-{3-chloro-6-[(2-oxopyrrolidin-1-yl)methyl]-5-pent-4-enoylpyridin-2-yl}piperidine-4-carboxylate

Prepared according to Example 1(g) from tert-butyl 1-{3-chloro-5-(fluorocarbonyl)-6-[(2-oxopyrrolidin-1-yl)methyl]pyridin-2-yl}piperidine-4-carboxylate (Example 30 (h)) (1.5 g, 2.8 mmol) by using cyclopropyl methyl magnesium bromide in place of methylmagnesium chloride to give tert-butyl 1-{3-chloro-6-[(2-oxopyrrolidin-1-yl)methyl]-5-pent-4-enoylpyridin-2-yl}piperidine-4-carboxylate. Yield: 1.7 g (89%).

### (b) 1-{3-Chloro-6-[(2-oxopyrrolidin-1-yl)methyl]-5-pent-4-enoylpyridin-2-yl}piperidine-4-carboxylic acid

Prepared according to Example 1(h) from tert-butyl 1-{3-chloro-6-[(2-oxopyrrolidin-1-yl)methyl]-5-pent-4-enoylpyridin-2-yl}piperidine-4-carboxylate (1.19 g, 2.49 mmol) to give 1-{3-chloro-6-[(2-oxopyrrolidin-1-yl)methyl]-5-pent-4-enoylpyridin-2-yl}piperidine-4-carboxylic acid. Yield: 320 mg (31%).

¹H-NMR (400 MHz, CDCl₃) δ 1.81-1.92 (2H, m), 1.98-2.13 (4H, m), 2.41-2.52 (4H, m), 2.55-2.64 (1H, m), 2.93 (2H, t), 3.04-3.12 (2H, m), 3.49 (2H, t), 4.05-4.13 (2H, m), 4.82 (2H, s), 5.00-5.11 (2H, m), 5.81-5.94 (1H, m), 7.95 (1H, s).

MSm/z: 420 (M+1), 418 (M−1)

### (c) 1-{3-chloro-6-[(2-oxopyrrolidin-1-yl)methyl]-5-pent-4-enoylpyridin-2-yl}-N-{[(4-fluorophenyl)(methyl)amino]sulfonyl}piperidine-4-carboxamide

Prepared according to Example 13(d) from 1-{3-chloro-6-[(2-oxopyrrolidin-1-yl)methyl]-5-pent-4-enoylpyridin-2-yl}piperidine-4-carboxylic acid (Example 50(b)) (160 mg, 0.38 mmol) to give 1-{3-chloro-6-[(2-oxopyrrolidin-1-yl)methyl]-5-pent-4-enoylpyridin-2-yl}-N-{[(4-fluorophenyl)(methyl)amino]sulfonyl}piperidine-4-carboxamide. Yield: 137 mg (59%).

¹H-NMR (400 MHz, DMSO-d₆) d 1.51-1.67 (2H, m), 1.69-1.80 (2H, m), 1.91-2.01 (2H, m), 2.14-2.22 (1H, m), 2.23-2.30 (2H, m), 2.30-2.37 (2H, m), 2.85-2.97 (2H, m), 3.06 (2H, t), 3.32 (3H, s), 3.42 (2H, t), 3.99-4.13 (2H, m), 4.61 (2H, s), 4.94-5.01 (1H, m), 5.03-5.12 (1H, m), 5.79-5.93 (1H, m), 7.23-7.31 (2H, m), 7.33-7.41 (2H, m), 8.31 (1H, s), 11.62 (1H, s).

MSm/z: 606 (M+1), 604 (M−1)

GTPγS(IC₅₀ μM): 0.167

### Example 51

### N-(benzylsulfonyl)-1-{3-chloro-6-[(2-oxopyrrolidin-1-yl)methyl]-5-pent-4-enoylpyridin-2-yl}piperidine-4-carboxamide

Prepared according to Example 13(d) from 1-{3-chloro-6-[(2-oxopyrrolidin-1-yl)methyl]-5-pent-4-enoylpyridin-2-yl}piperidine-4-carboxylic acid (Example 50(b)) (160 mg, 0.38 mmol) to give N-(benzylsulfonyl)-1-{3-chloro-6-[(2-oxopyrrolidin-1-yl)methyl]-5-pent-4-enoylpyridin-2-yl}piperidine-4-carboxamide. Yield: 146 mg (67%).

¹H-NMR (400 MHz, DMSO-d₆) δ 1.57-1.72 (2H, m), 1.74-1.84 (2H, m), 1.95-2.03 (2H, m), 2.14-2.26 (1H, m), 2.26-2.35 (4H, m), 2.86-2.98 (2H, m), 3.07 (2H, t), 3.43 (2H, t), 4.03-4.12 (2H, m), 4.61 (2H, s), 4.70 (2H, s), 4.98 (1H, m), 5.08 (1H, m), 5.86 (1H, m), 7.28-7.30 (2H, m), 7.38-7.44 (3H, m), 8.31 (1H, s), 8.31 (1H, s).

MSm/z: 573 (M+1), 571 (M−1)

GTPγS(IC₅₀ μM): 0.069

### Example 52

### 1-{5-butyryl-3-chloro-6-[(2-oxopyrrolidin-1-yl)methyl]pyridin-2-yl}-N-[(cyclohexylmethyl)sulfonyl]piperidine-4-carboxamide

Prepared according to Example 13(d) from 1-{5-butyryl-3-chloro-6-[(2-oxopyrrolidin-1-yl)methyl]pyridin-2-yl}piperidine-4-carboxylic acid (Example 30(j)) (129 mg, 0.32 mmol) and 1-cyclohexylmethanesulfonamide (67 mg, 0.38 mmol) to give 1-{5-butyryl-3-chloro-6-[(2-oxopyrrolidin-1-yl)methyl]pyridin-2-yl}-N-[(cyclohexylmethyl)sulfonyl]piperidine-4-carboxamide. Yield: 132 mg (74%).

¹H-NMR (400 MHz, CDCl₃) δ 0.99 (3H, t), 1.04-1.36 (5H, m), 1.60-1.96 (11H, m), 1.96-2.14 (3H, m), 2.45-2.55 (3H, m), 2.81 (2H, t), 2.89-2.99 (2H, m), 3.33 (2H, d), 3.46 (2H, t), 4.03-4.10 (2H, m), 4.83 (2H, s), 7.97 (1H, s), 9.63 (1H, s).

MSm/z: 567 (M+1), 565 (M−1).

GTPγS(IC₅₀ μM): 0.231

### Example 53

### 1-{5-butyryl-3-chloro-6-[(2-oxopyrrolidin-1-yl)methyl]pyridin-2-yl}-N-[(2,4-difluorobenzyl)sulfonyl]piperidine-4-carboxamide

Prepared according to Example 13(d) from 1-{5-butyryl-3-chloro-6-[(2-oxopyrrolidin-1-yl)methyl]pyridin-2-yl}piperidine-4-carboxylic acid (Example 30(j)) (129 mg, 0.32 mmol) and 1-(2,4-difluorophenyl)methanesulfonamide (78.6 mg, 0.38 mmol) to give 1-{5-butyryl-3-chloro-6-[(2-oxopyrrolidin-1-yl)methyl]pyridin-2-yl}-N-[(2,4-difluorobenzyl)sulfonyl]piperidine-4-carboxamide. Yield: 159 mg (84%).

¹H-NMR (400 MHz, CDCl₃) δ 0.97 (3H, t), 1.70 (2H, sextet), 1.79-1.90 (4H, m), 1.98-2.08 (2H, m), 2.28-2.36 (2H, m), 2.45-2.55 (1H, m), 2.76-2.82 (2H, m), 2.82-2.92 (2H, m), 3.36-3.45 (2H, m), 3.98-4.08 (2H, m), 4.65 (2H, s), 4.74 (2H, s), 6.82-6.95 (2H, m), 7.34-7.42 (1H, m), 7.94 (1H, s), 10.40 (1H, s).

MSm/z: 597 (M+1), 595 (M−1).

GTPγS(IC₅₀ μM): 0.032

### Example 54

### 1-{3-chloro-6-[(2-oxopyrrolidin-1-yl)methyl]-5-pentanoylpyridin-2-yl}-N-[(cyclopentylmethyl)sulfonyl]piperidine-4-carboxamide

### (a) tert-Butyl 1-{3-chloro-6-[(2-oxopyrrolidin-1-yl)methyl]-5-pentanoylpyridin-2-yl}piperidine-4-carboxylate

Prepared according to Example 42(b) from tert-butyl 1-{3-chloro-5-(fluorocarbonyl)-6-[(2-oxopyrrolidin-1-yl)methyl]pyridin-2-yl}piperidine-4-carboxylate (Example 30(h)) (1.96 g, 4.46 mmol) to give tert-butyl 1-{3-chloro-6-[(2-oxopyrrolidin-1-yl)methyl]-5-pentanoylpyridin-2-yl}piperidine-4-carboxylate. Yield: 770 mg (36%).

¹H-NMR (500 MHz, CDCl₃) δ 0.95 (3H, t), 1.32-1.44 (2H, m), 1.45 (9H, s), 1.66 (2H, m), 1.75-1.85 (2H, m), 1.92-1.99 (2H, m), 2.08 (2H, m), 2.40-2.48 (3H, m), 2.81 (2H, t), 3.02 (2H, m), 3.50 (2H, t), 4.06 (2H, m), 4.80 (2H, s), 7.93 (1H, s).

MSm/z: 478 (M+1).

### (b) 1-{3-Chloro-6-[(2-oxopyrrolidin-1-yl)methyl]-5-pentanoylpyridin-2-yl}piperidine-4-carboxylic acid

tert-Butyl 1-{3-chloro-6-[(2-oxopyrrolidin-1-yl)methyl]-5-pentanoylpyridin-2-yl}piperidine-4-carboxylate (770 mg, 1.61 mmol) was dissolved in 4M HCl (10 mL in dioxane), the reaction mixture was stirred at r.t for 2 h and then 1 h at 50° C. The solvent was concentrated in vacuo and the crude product was used in the next step without purification.

MSm/z: 422 (M+1), 420 (M−1).

### (c) 1-{3-chloro-6-[(2-oxopyrrolidin-1-yl)methyl]-5-pentanoylpyridin-2-yl}-N-[(cyclopentylmethyl)sulfonyl]piperidine-4-carboxamide

Prepared according to Example 13(d) from 1-{3-chloro-6-[(2-oxopyrrolidin-1-yl)methyl]-5-pentanoylpyridin-2-yl}piperidine-4-carboxylic acid (183 mg, 0.4 mmol) and 1-cyclopentylmethanesulfonamide (73 mg, 0.45 mmol) to give 1-{3-chloro-6-[(2-oxopyrrolidin-1-yl)methyl]-5-pentanoylpyridin-2-yl}-N-[(cyclopentylmethyl)sulfonyl]piperidine-4-carboxamide. Yield: 58 mg (26%).

¹H-NMR (500 MHz, DMSO-d₆) 0.89 (3H, t), 1.20-1.36 (4H, m), 1.45-1.67 (8H, m), 1.81-1.89 (4H, m), 1.93-2.02 (2H, m), 2.15 (1H, m), 2.26 (2H, t), 2.58 (1H, m), 2.91-2.98 (4H, m), 3.38-3.45 (4H, m), 4.01-4.10 (3H, m), 4.60 (2H, s), 8.28 (1H, s).

MSm/z: 567 (M+1), 565 (M−1).

GTPγS(IC₅₀ μM): 0.938

### Example 55

### 1-{3-chloro-6-[(2-oxopyrrolidin-1-yl)methyl]-5-pentanoylpyridin-2-yl}-N-[(2,4-difluorobenzyl)sulfonyl]piperidine-4-carboxamide

Prepared according to Example 13(d) from 1-{3-chloro-6-[(2-oxopyrrolidin-1-yl)methyl]-5-pentanoylpyridin-2-yl}piperidine-4-carboxylic acid (Example 54(b)) (183 mg, 0.4 mmol) and 1-(2,4-difluorophenyl)methanesulfonamide (94 mg, 0.45 mmol) to give 1-{3-chloro-6-[(2-oxopyrrolidin-1-yl)methyl]-5-pentanoylpyridin-2-yl}-N-[(2,4-difluorobenzyl)sulfonyl]piperidine-4-carboxamide. Yield: 96 mg (39%).

¹H-NMR (500 MHz, CD₃OD) 0.95 (3H, t), 1.35-1.44 (2H, m), 1.64 (2H, m), 1.78-1.92 (4H, m), 2.13 (2H, m), 2.46-2.52 (3H, m), 2.92 (2H, t), 2.99 (2H, m), 3.55 (2H, t), 4.17 (2H, m), 4.72 (2H, s), 4.75 (2H, s), 7.01-7.08 (2H, m), 7.45-7.51 (1H, m), 8.19 (1H, s).

MSm/z: 611 (M+1), 609 (M−1).

GTPγS(IC₅₀ μM): 0.226

### Example 56

### 1-{3-chloro-6-[(2-oxopyrrolidin-1-yl)methyl]-5-pentanoylpyridin-2-yl}-N-{[(4-fluorophenyl)(methyl)amino]sulfonyl}piperidine-4-carboxamide

Prepared according to Example 13(d) from 1-{3-chloro-6-[(2-oxopyrrolidin-1-yl)methyl]-5-pentanoylpyridin-2-yl}piperidine-4-carboxylic acid (Example 54(b)) (183 mg, 0.4 mmol) and N-(4-fluorophenyl)-N-methylsulfamide (92 mg, 0.45 mmol) to give 1-{3-chloro-6-[(2-oxopyrrolidin-1-yl)methyl]-5-pentanoylpyridin-2-yl}-N-{[(4-fluorophenyl)(methyl)amino]sulfonyl}piperidine-4-carboxamide. Yield: 91 mg (37%).

¹H-NMR (500 MHz, DMSO-d₆) 0.90 (3H, t), 1.32 (2H, m), 1.50-1.65 (4H, m), 1.71-1.78 (2H, m), 1.97 (2H, m), 2.26 (2H, t), 2.46 (1H, m), 2.90 (2H, m), 2.94 (2H, t), 3.32 (3H, s), 3.42 (2H, t), 4.05 (2H, m), 4.60 (2H, s), 7.27 (2H, m), 7.36-7.39 (2H, m), 8.28 (1H, s).

MSm/z: 608 (M+1), 606 (M−1).

GTPγS(IC₅₀ μM): 0.247

### Example 57

### N-(benzylsulfonyl)-1-{3-chloro-6-[(2-oxopyrrolidin-1-yl)methyl]-5-pentanoylpyridin-2-yl}piperidine-4-carboxamide

Prepared according to Example 13(d) from 1-{3-chloro-6-[(2-oxopyrrolidin-1-yl)methyl]-5-pentanoylpyridin-2-yl}piperidine-4-carboxylic acid (Example 54(b)) (183 mg, 0.4 mmol) and N-phenylsulfamide (78 mg, 0.46 mmol) to give N-(benzylsulfonyl)-1-{3-chloro-6-[(2-oxopyrrolidin-1-yl)methyl]-5-pentanoylpyridin-2-yl}piperidine-4-carboxamide. Yield: 71 mg (31%).

¹H-NMR (500 MHz, CD₃OD) 0.95 (3H, t), 1.35-1.44 (2H, m), 1.64 (2H, m), 1.74-1.85 (4H, m), 2.12 (2H, m), 2.38-2.48 (3H, m), 2.91 (2H, t), 2.95 (2H, m), 3.55 (2H, t), 4.15 (2H, m), 4.66 (2H, s), 4.75 (2H, s), 7.33-7.40 (5H, m), 8.19 (1H, s).

MSm/z: 575 (M+1), 573 (M−1).

GTPγS(IC₅₀ μM): 0.079

### Example 58

### N-(benzylsulfonyl)-1-{3-cyano-6-[(2-oxopyrrolidin-1-yl)methyl]-5-(4,4,4-trifluorobutanoyl)pyridin-2-yl}piperidine-4-carboxamide

Prepared according to Example 13(d) from 1-{3-cyano-6-[(2-oxopyrrolidin-1-yl)methyl]-5-(4,4,4-trifluorobutanoyl)pyridin-2-yl}piperidine-4-carboxylic acid (Example 42(c)) (30 mg, 0.07 mmol) and N-phenylsulfamide (18 mg, 0.06 mmol) to give N-(benzylsulfonyl)-1-{3-cyano-6-[(2-oxopyrrolidin-1-yl)methyl]-5-(4,4,4-trifluorobutanoyl)pyridin-2-yl}piperidine-4-carboxamide. Yield: 8 mg (28%).

¹H-NMR (400 MHz, CDCl₃) δ 1.61-1.81 (4H, m), 1.94-2.04 (2H, m), 2.24-2.30 (2H, m), 2.37-2.57 (3H, m), 2.98-3.05 (2H, m), 3.08-3.20 (2H, m), 3.34-3.40 (2H, m), 4.37-4.46 (3H, m), 4.50-4.55 (2H, m), 4.73 (2H, s), 7.23-7.32 (5H, m), 8.12 (1H, s)

GTPγS(IC₅₀ μM): 0.084

### Example 59

### N-[(4-chlorobenzyl)sulfonyl]-1-{3-chloro-6-[(2-oxopyrrolidin-1-yl)methyl]-5-pentanoylpyridin-2-yl}piperidine-4-carboxamide

Prepared according to Example 13(d) from 1-{3-chloro-6-[(2-oxopyrrolidin-1-yl)methyl]-5-pentanoylpyridin-2-yl}piperidine-4-carboxylic acid (Example 54(b)) (66 mg, 0.16 mmol) to give N-[(4-chlorobenzyl)sulfonyl]-1-{3-chloro-6-[(2-oxopyrrolidin-1-yl)methyl]-5-pentanoylpyridin-2-yl}piperidine-4-carboxamide. Yield: 82 mg (86%).

¹H-NMR (400 MHz, CDCl₃) δ 0.94 (3H, t), 1.32-1.42 (2H, m), 1.61-1.69 (2H, m), 1.75-1.84 (3H, m), 1.99-2.09 (2H, m), 2.24-2.33 (3H, m), 2.42-2.51 (1H, m), 2.78-2.90 (4H, m), 3.38-3.44 (2H, m), 3.98-4.07 (2H, m), 4.59 (2H, s), 4.75 (2H, s), 7.25-7.36 (4H, m), 7.95 (1H, s), 10.30 (1H, s).

GTPγS(IC₅₀ μM): 0.132

### Example 60

### 1-{3-chloro-6-[(2-oxopyrrolidin-1-yl)methyl]-5-pentanoylpyridin-2-yl}-N-[(1-phenylethyl)sulfonyl]piperidine-4-carboxamide

Prepared according to Example 13(d) from 1-{3-chloro-6-[(2-oxopyrrolidin-1-yl)methyl]-5-pentanoylpyridin-2-yl}piperidine-4-carboxylic acid (Example 54(b)) (66 mg, 0.16 mmol) to give 1-{3-chloro-6-[(2-oxopyrrolidin-1-yl)methyl]-5-pentanoylpyridin-2-yl}-N-[(1-phenylethyl)sulfonyl]piperidine-4-carboxamide. Yield: 26 mg (28%).

¹H-NMR (600 MHz, CDCl₃) δ 0.94 (3H, t), 1.34-1.41 (2H, m), 1.62-1.70 (4H, m), 1.80 (3H, d), 1.87-1.95 (2H, m), 2.01-2.07 (2H, m), 2.33-2.38 (3H, m), 2.79-2.90 (4H, m), 3.40-3.45 (2H, m), 3.97-4.04 (2H, m), 4.76 (2H, s), 4.89 (1H, q), 7.33-7.41 (5H, m), 7.94 (1H, s), 9.25 (1H, s).

GTPγS(IC₅₀ μM): 0.268

### Example 61

### 1-{3-cyano-6-[(2-oxopyrrolidin-1-yl)methyl]-5-pentanoylpyridin-2-yl}-N-[(1-phenylethyl)sulfonyl]piperidine-4-carboxamide

Prepared according to Example 13(d) from 1-{3-cyano-6-[(2-oxopyrrolidin-1-yl)methyl]-5-pentanoylpyridin-2-yl}piperidine-4-carboxylic acid (Example 17(b)) (120 mg, 0.29 mmol) to give 1-{3-cyano-6-[(2-oxopyrrolidin-1-yl)methyl]-5-pentanoylpyridin-2-yl}-N-[(1-phenylethyl)sulfonyl]piperidine-4-carboxamide. Yield: 122 mg (72%).

¹H-NMR (400 MHz, CDCl₃) δ 0.95 (3H, t), 1.18-1.22 (1H, m), 1.33-1.43 (2H, m), 1.62-1.87 (7H, m), 2.02-2.11 (3H, m), 2.33-2.39 (2H, m), 2.41-2.51 (1H, m), 2.81 (2H, t), 3.12-3.23 (2H, m), 3.40-3.47 (2H, m), 4.38-4.49 (2H, m), 4.78-4.91 (3H, m), 7.33-7.40 (5H, m), 8.20 (1H, s), 9.49-9.59 (1H, m).

GTPγS(IC₅₀ μM): 0.033.

### Example 62

### 1-{3-cyano-5-(5-ethyl-1,3-oxazol-2-yl)-6-[(2-oxopyrrolidin-1-yl)methyl]pyridin-2-yl}-N-{[(4-fluorophenyl)(methyl)amino]sulfonyl}piperidine-4-carboxamide

### (a) tert-Butyl 1-{3-cyano-5-[(2-hydroxybutyl)carbamoyl]-6-[(2-oxopyrrolidin-1-yl)methyl]pyridin-2-yl}piperidine-4-carboxylate

tert-Butyl 1-{3-cyano-5-(fluorocarbonyl)-6-[(2-oxopyrrolidin-1-yl)methyl]pyridin-2-yl}piperidine-4-carboxylate (Example 42 (a)) (1 g, 2.3 mmol) was dissolved in DCM (10 mL), 1-amino-2-butanol (0.3 mL, 3.5 mmol) and DIPEA 0.8 mL, 4.65 mmol) were added at 5° C. The reaction mixture was stirred at 5° C. for 10 min, the crude material was loaded directly onto silica and purified by flash column chromatography (EtOAc:EtOH 100:0 to 90:10) to give tert-butyl 1-{3-cyano-5-[(2-hydroxybutyl)carbamoyl]-6-[(2-oxopyrrolidin-1-yl)methyl]pyridin-2-yl}piperidine-4-carboxylate. Yield: 0.96 g (82%).

¹H-NMR (600 MHz, DMSO-d₆) δ 0.86 (3H, t), 1.24-1.32 (1H, m), 1.39-1.46 (1H, m), 1.52 (2H, qd), 1.86 (2H, dd), 1.94 (2H, quintet), 2.23 (2H, t), 2.55 (1H, tt), 3.03-3.10 (1H, m), 3.14-3.24 (3H, m), 3.38 (2H, t), 3.44-3.50 (1H, m), 4.24 (2H, d), 4.59 (2H, s), 4.68 (1H, d), 8.16 (1H, s), 8.34 (1H, t).

MSm/z: 500 (M+1), 498 (M−1).

### (b) tert-Butyl 1-{3-cyano-5-[(2-oxopropyl)carbamoyl]-6-[(2-oxopyrrolidin-1-yl)methyl]pyridin-2-yl}piperidine-4-carboxylate

tert-Butyl 1-{3-cyano-5-[(2-hydroxybutyl)carbamoyl]-6-[(2-oxopyrrolidin-1-yl)methyl]pyridin-2-yl}piperidine-4-carboxylate (Example 62 (a)) (250 mg, 0.5 mmol) was dissolved in DCM (3.7 mL), Dess Martin periodinane (1.3 mL, 0.6 mmol) was added and the reaction mixture was stirred over night. NaHCO₃(aq)(5 mL) was added and the precipitated was filtered, the organic phase was separated, dried and concentrated in vacuo to give the crude material. Methyl tetrahydrofurane (5 mL) was added and the solid was filtered, the organic solution was concentrated in vacuo to give tert-butyl 1-{3-cyano-5-[(2-oxopropyl)carbamoyl]-6-[(2-oxopyrrolidin-1-yl)methyl]pyridin-2-yl}piperidine-4-carboxylate.

¹H-NMR (600 MHz, CDCl₃) δ 1.03 (3H, t), 1.36 (9H, s), 1.65-1.72 (2H, m), 1.90 (2H, dd), 2.02 (2H, quintet), 2.32 (2H, t), 2.39-2.47 (3H, m), 3.12-3.19 (2H, m), 3.65 (2H, t), 4.17 (2H, d), 4.28 (2H, d), 4.64 (2H, s), 7.97 (1H, s), 8.83 (1H, t).

MSm/z: 498 (M+1), 496 (M−1).

### (c) tert-Butyl 1-{3-cyano-5-(5-ethyl-1,3-oxazol-2-yl)-6-[(2-oxopyrrolidin-1-yl)methyl]pyridin-2-yl}piperidine-4-carboxylate

tert-Butyl 1-{3-cyano-5-[(2-oxopropyl)carbamoyl]-6-[(2-oxopyrrolidin-1-yl)methyl]pyridin-2-yl}piperidine-4-carboxylat (154 mg, 0.26 mmol) was dissolved in DCM (3 mL), pyridine (0.42 mL, 5.14 mmol), DMAP (3.14 mg, 0.026 mmol) and trichloroacetyl chloride (0.26 mL, 2.3 mmol) were added. The reaction mixture was stirred at r.t for 10 min, 1N HCl (5 mL) and DCM (2 mL) were added, the organic phase was loaded directly onto silica and purified by column chromatography, to give tert-butyl 1-{3-cyano-5-(5-ethyl-1,3-oxazol-2-yl)-6-[(2-oxopyrrolidin-1-yl)methyl]pyridin-2-yl}piperidine-4-carboxylate. Yield: 88 mg (71%)

¹H-NMR (600 MHz, CDCl₃) δ 1.28 (3H, t), 1.44 (9H, s), 1.74-1.82 (2H, m), 1.95-2.00 (2H, m), 2.08 (2H, quintet), 2.45 (2H, t), 2.50 (1H, tt), 2.72 (2H, q), 3.26 (2H, ddd), 3.50 (2H, t), 4.39 (2H, dt), 4.98 (2H, s), 6.81 (1H, s), 8.30 (1H, s).

MSm/z: 480 (M+1).

### (d) 1-{3-Cyano-5-(5-ethyl-1,3-oxazol-2-yl)-6-[(2-oxopyrrolidin-1-yl)methyl]pyridin-2-yl}piperidine-4-carboxylic acid

Prepared according to Example 1(h) from tert-butyl 1-{3-cyano-5-(5-ethyl-1,3-oxazol-2-yl)-6-[(2-oxopyrrolidin-1-yl)methyl]pyridin-2-yl}piperidine-4-carboxylate (88 mg, 0.183 mmol) to give 1-{3-cyano-5-(5-ethyl-1,3-oxazol-2-yl)-6-[(2-oxopyrrolidin-1-yl)methyl]pyridin-2-yl}piperidine-4-carboxylic acid. Yield: 76 mg (98%).

¹H-NMR (600 MHz, CDCl₃) δ 1.31 (3H, t), 1.79-1.89 (2H, m), 2.01-2.09 (2H, m), 2.14 (2H, quintet), 2.60 (2H, t), 2.64-2.71 (1H, m), 2.75 (2H, q), 3.32 (2H, t), 3.55 (2H, t), 4.41 (2H, d), 5.01 (2H, s), 6.87 (1H, s), 8.31 (1H, s), 10.28 (1H, s).

MSm/z: 424 (M+1), 422 (M−1).

### (e) 1-{3-cyano-5-(5-ethyl-1,3-oxazol-2-yl)-6-[(2-oxopyrrolidin-1-yl)methyl]pyridin-2-yl}-N-{[(4-fluorophenyl)(methyl)amino]sulfonyl}piperidine-4-carboxamide

Prepared according to Example 1(i) from 1-{3-cyano-5-(5-ethyl-1,3-oxazol-2-yl)-6-[(2-oxopyrrolidin-1-yl)methyl]pyridin-2-yl}piperidine-4-carboxylic acid (32 mg, 0.076 mmol) to give 1-{3-cyano-5-(5-ethyl-1,3-oxazol-2-yl)-6-[(2-oxopyrrolidin-1-yl)methyl]pyridin-2-yl}-N-{[(4-fluorophenyl)(methyl)amino]sulfonyl}piperidine-4-carboxamide. Yield: 19 mg (41%).

¹H-NMR (600 MHz, CDCl₃) δ 1.30 (3H, t), 1.73-1.85 (4H, m), 2.04-2.10 (2H, m), 2.39 (2H, t), 2.44-2.50 (1H, m), 2.74 (2H, q), 3.08-3.13 (2H, m), 3.45 (3H, s), 3.45-3.48 (2H, m), 4.38-4.43 (2H, m), 4.97 (2H, s), 6.84 (1H, s), 7.03-7.07 (2H, m), 7.31-7.34 (2H, m), 8.32 (1H, s), 9.37 (1H, s).

MSm/z: 610 (M+1), 608 (M−1).

GTPγS(IC₅₀ μM): 0.03.

### Example 63

### N-(benzylsulfonyl)-1-{3-cyano-5-(5-ethyl-1,3-oxazol-2-yl)-6-[(2-oxopyrrolidin-1-yl)methyl]pyridin-2-yl}piperidine-4-carboxamide

Prepared according to Example 1(i) from 1-{3-cyano-5-(5-ethyl-1,3-oxazol-2-yl)-6-[(2-oxopyrrolidin-1-yl)methyl]pyridin-2-yl}piperidine-4-carboxylic acid (32 mg, 0.076 mmol) to give N-(benzylsulfonyl)-1-{3-cyano-5-(5-ethyl-1,3-oxazol-2-yl)-6-[(2-oxopyrrolidin-1-yl)methyl]pyridin-2-yl}piperidine-4-carboxamide. Yield: 20 mg (46%).

¹H-NMR (400 MHz, CDCl₃) δ 1.29 (3H, t), 1.70-1.82 (2H, m), 1.83-1.92 (2H, m), 2.13 (2H, quintet), 2.44-2.53 (1H, m), 2.57 (2H, t), 2.78 (2H, q), 3.16-3.28 (2H, m), 3.62 (2H, t), 4.53 (2H, d), 4.57 (2H, s), 4.75 (2H, s), 7.13 (1H, s), 7.24-7.38 (5H, m), 8.28 (1H, s).

MSm/z: 577 (M+1), 575 (M−1).

GTPγS(IC₅₀ μM): 0.021.

### Example 64

### 1-{3-chloro-5-(5-ethyl-1,3-oxazol-2-yl)-6-[(2-oxopyrrolidin-1-yl)methyl]pyridin-2-yl}-N-{[(4-fluorophenyl)(methyl)amino]sulfonyl}piperidine-4-carboxamide

### (a) tert-Butyl 1-{3-chloro-5-[(2-hydroxybutyl)carbamoyl]-6-[(2-oxopyrrolidin-1-yl)methyl]pyridin-2-yl}piperidine-4-carboxylate

Prepared according to Example 62(a) from tert-butyl 1-{3-chloro-5-(fluorocarbonyl)-6-[(2-oxopyrrolidin-1-yl)methyl]pyridin-2-yl}piperidine-4-carboxylate (Example 30 (h)) (1 g, 2.27 mmol) to give tert-butyl 1-{3-chloro-5-[(2-hydroxybutyl)carbamoyl]-6-[(2-oxopyrrolidin-1-yl)methyl]pyridin-2-yl}piperidine-4-carboxylate. Yield: 640 mg (55%)

¹H-NMR (600 MHz, DMSO-d₆) δ 0.87 (3H, t), 1.24-1.32 (1H, m), 1.38 (9H, s), 1.39-1.47 (1H, m), 1.55-1.65 (2H, m), 1.81-1.87 (2H, m), 1.94 (2H, quintet), 2.23 (2H, t), 2.45 (1H, tt), 2.90 (2H, t), 3.05-3.12 (1H, m), 3.22 (1H, dt), 3.39 (2H, t), 3.45-3.52 (1H, m), 3.77 (2H, d), 4.54 (2H, s), 4.67 (1H, d), 7.86 (1H, s), 8.40 (1H, t).

MSm/z: 509 (M+1), 507 (M−1).

### (b) tert-Butyl 1-{3-chloro-5-[(2-oxobutyl)carbamoyl]-6-[(2-oxopyrrolidin-1-yl)methyl]pyridin-2-yl}piperidine-4-carboxylate

Prepared according to Example 62(b) from tert-butyl 1-{3-chloro-5-[(2-hydroxybutyl)carbamoyl]-6-[(2-oxopyrrolidin-1-yl)methyl]pyridin-2-yl}piperidine-4-carboxylate (200 mg, 0.393 mmol) to give tert-butyl 1-{3-chloro-5-[(2-oxobutyl)carbamoyl]-6-[(2-oxopyrrolidin-1-yl)methyl]pyridin-2-yl}piperidine-4-carboxylate. Yield: 115 mg (58%).

¹H-NMR (600 MHz, CDCl₃) δ 1.07 (3H, t), 1.42 (9H, s), 1.79 (2H, ddd), 1.89-1.96 (2H, m), 2.06 (2H, quintet), 2.34 (2H, t), 2.38 (1H, tt), 2.48 (2H, q), 2.84-2.93 (2H, m), 3.81 (2H, t), 3.87 (2H, d), 4.24 (2H, d), 4.65 (2H, s), 7.76 (1H, s), 9.27 (1H, t).

MSm/z: 507 (M+1), 505 (M−1)

### (c) tert-Butyl 1-{3-chloro-5-(5-ethyl-1,3-oxazol-2-yl)-6-[(2-oxopyrrolidin-1-yl)methyl]pyridin-2-yl}piperidine-4-carboxylate

Prepared according to Example 62(c) from tert-butyl 1-{3-chloro-5-[(2-oxobutyl)carbamoyl]-6-[(2-oxopyrrolidin-1-yl)methyl]pyridin-2-yl}piperidine-4-carboxylate (708 mg, 1.4 mmol) to give tert-butyl 1-{3-chloro-5-(5-ethyl-1,3-oxazol-2-yl)-6-[(2-oxopyrrolidin-1-yl)methyl]pyridin-2-yl}piperidine-4-carboxylate. Yield: 690 mg (100%).

¹H-NMR (400 MHz, CDCl₃) δ 1.28 (3H, t), 1.45 (9H, s), 1.75-1.88 (2H, m), 1.91-1.99 (2H, m), 2.06 (2H, quintet), 2.40 (1H, tt), 2.46 (2H, t), 2.72 (2H, qd), 2.89-2.98 (2H, m), 3.50 (2H, t), 3.93 (2H, d), 4.93 (2H, s), 6.83 (1H, s), 8.09 (1H, s).

MSm/z: 489 (M+1)

### (d) 1-{3-Chloro-5-(5-ethyl-1,3-oxazol-2-yl)-6-[(2-oxopyrrolidin-1-yl)methyl]pyridin-2-yl}piperidine-4-carboxylic acid

Prepared according to Example 1(h) from tert-butyl 1-{3-chloro-5-(5-ethyl-1,3-oxazol-2-yl)-6-[(2-oxopyrrolidin-1-yl)methyl]pyridin-2-yl}piperidine-4-carboxylate (650 mg, 1.33 mmol) to give 1-{3-chloro-5-(5-ethyl-1,3-oxazol-2-yl)-6-[(2-oxopyrrolidin-1-yl)methyl]pyridin-2-yl}piperidine-4-carboxylic acid. Yield: 331 mg (56%).

¹H-NMR (600 MHz, DMSO-d₆) δ 1.22 (3H, t), 1.57-1.69 (2H, m), 1.84-1.92 (2H, m), 1.97 (2H, quintet), 2.26 (2H, t), 2.45-2.52 (1H, m), 2.72 (2H, q), 2.96 (2H, t), 3.44 (2H, t), 3.86 (2H, d), 4.77 (2H, s), 7.03 (1H, s), 8.10 (1H, s).

MSm/z: 433 (M+1), 431 (M−1)

### (e) 1-{3-chloro-5-(5-ethyl-1,3-oxazol-2-yl)-6-[(2-oxopyrrolidin-1-yl)methyl]pyridin-2-yl}-N-{[(4-fluorophenyl)(methyl)amino]sulfonyl}piperidine-4-carboxamide

Prepared in a similarly was as Example 13(d) from 1-{3-chloro-5-(5-ethyl-1,3-oxazol-2-yl)-6-[(2-oxopyrrolidin-1-yl)methyl]pyridin-2-yl}piperidine-4-carboxylic acid (50 mg, 0.116 mmol) to give 1-{3-chloro-5-(5-ethyl-1,3-oxazol-2-yl)-6-[(2-oxopyrrolidin-1-yl)methyl]pyridin-2-yl}-N-{[(4-fluorophenyl)(methyl)amino]sulfonyl}piperidine-4-carboxamide. Yield: 22 mg (31%).

¹H-NMR (600 MHz, CDCl₃) δ 1.29 (3H, t), 1.75-1.85 (4H, m), 2.04 (2H, quintet), 2.34-2.41 (3H, m), 2.73 (2H, q), 2.75-2.81 (2H, m), 3.43 (2H, t), 3.46 (3H, s), 3.91 (2H, d), 4.92 (2H, s), 6.83 (1H, s), 7.02-7.07 (2H, m), 7.32-7.37 (2H, m), 8.11 (1H, s), 9.47 (1H, s).

MSm/z: 619 (M+1), 617 (M−1).

GTPγS(IC₅₀ μM): 0.45.

### Example 65

### N-(benzylsulfonyl)-1-{3-chloro-5-(5-ethyl-1,3-oxazol-2-yl)-6-[(2-oxopyrrolidin-1-yl)methyl]pyridin-2-yl}piperidine-4-carboxamide

Prepared in a similar way as Example 13(d) from 1-{3-chloro-5-(5-ethyl-1,3-oxazol-2-yl)-6-[(2-oxopyrrolidin-1-yl)methyl]pyridin-2-yl}piperidine-4-carboxylic acid (Example 64 (d)) (50 mg, 0.116 mmol) to give N-(benzylsulfonyl)-1-{3-chloro-5-(5-ethyl-1,3-oxazol-2-yl)-6-[(2-oxopyrrolidin-1-yl)methyl]pyridin-2-yl}piperidine-4-carboxamide. Yield: 27 mg (40%).

¹H-NMR (600 MHz, CDCl₃) δ 1.29 (3H, t), 1.77-1.88 (4H, m), 2.02 (2H, quintet), 2.31 (2H, t), 2.37-2.45 (1H, m), 2.73 (2H, q), 2.76-2.84 (2H, m), 3.42 (2H, t), 3.91 (2H, d), 4.64 (2H, s), 4.91 (2H, s), 6.83 (1H, s), 7.32-7.38 (5H, m), 8.11 (1H, s), 9.62 (1H, br s).

MSm/z: 586 (M+1), 584 (M−1).

GTPγS(IC₅₀ μM): 0.17.

### Example 66

### N-(benzylsulfonyl)-1-{5-butyryl-3-chloro-6-[(2-oxopiperidin-1-yl)methyl]pyridin-2-yl}piperidine-4-carboxamide

Prepared essentially according to the procedures described in Example 30(e) to (k) using 6-methoxy-2,3,4,5-tetrahydro pyridine instead of 5-methoxy-3,4-dihydro-2H-pyrrole in step 30(e).

¹H NMR (400 MHz, DMSO-d₆) δ 0.91 (3H, t, J=7.4 Hz), 1.54-1.70 (4H, m), 1.72-1.85 (6H, m), 2.21-2.28 (2H, m), 2.45-2.55 (1H, m), 2.87-2.98 (4H, m), 3.33-3.40 (2H, m), 4.04-4.14 (2H, m), 4.66 (2H, s), 4.69 (2H, s), 7.26-7.32 (2H, m), 7.37-7.45 (3H, m), 8.26 (1H, s), 11.55 (1H, s)

MSm/z: 575 (M+1), 573 (M−1)

GTPγS(IC₅₀ μM): 0.252

### Example 67

### 1-{5-Butyryl-3-chloro-6-[(2-oxopiperidin-1-yl)methyl]pyridin-2-yl}-N-{[(4-fluorophenyl)(methyl)amino]sulfonyl}piperidine-4-carboxamide

Prepared essentially according to the procedures described in Example 30(e) to (k) using 6-methoxy-2,3,4,5-tetrahydro pyridine instead of 5-methoxy-3,4-dihydro-2H-pyrrole in step 30(e) and replacing 1-phenylmethylsulfonamid with (N-(4-fluorophenyl)-N-methylsulfamide in step 30(k).

¹H NMR (400 MHz, DMSO-d₆) δ 0.91 (3H, t, J=7.4 Hz), 1.54-1.65 (4H, m), 1.69-1.82 (6H, m), 2.18-2.29 (2H, m), 2.44-2.54 (1H, m), 2.85-2.97 (4H, m), 3.32 (3H, s, under water peak), 3.34-3.39 (1H, m), 3.99-4.12 (2H, m), 4.66 (3H, s), 7.22-7.29 (2H, m), 7.33-7.40 (2H, m), 8.25 (1H, s), 11.61 (1H, s).

MSm/z: 608 (M+1), 606 (M−1)

GTPγS(IC₅₀ μM): 0.445

### Example 68

### 1-{5-Butyryl-3-cyano-6-[(2-oxopyrrolidin-1-yl)methyl]pyridin-2-yl}-N-[(1-phenylethyl)sulfonyl]piperidine-4-carboxamide

A mixture of 1-{5-butyryl-3-cyano-6-[(2-oxopyrrolidin-1-yl)methyl]pyridin-2-yl}piperidine-4-carboxylic acid (150 mg, 0.376 mmol), TBTU (145 mg; 0.451 mmol) and DIPEA (0.197 mL; 1.13 mmol) were dissolved in DMF (4 mL) and stirred for 1 h at r.t. 1-phenylethanesulfonamide (76.7 mg, 0.414 mmol) (See Baskin, J. et al, Tetrahedron Letters, 2002, pp. 8479-84) was then added to the reaction mixture and the stirring was continured over night. LCMS showed some of the starting acid left. Therfore, PyBrop (175 mg, 0.376 mmol) was added. After 6 h an additional amount of 1-phenylethanesulfonamide was added (35 mg; 0.19 mmol) and the mixture was left at r.t. over night. LCMS shows starting material:product/1:1. The mixture was transferred to a micro vial and heated in a microwave oven single node heating twice at 80° C. for 30 minutes. The mixture was diluted with DCM and washed with sat. NaHCO₃(aq). The aqueous phase was extracted with DCM and the combined organic phases were filtered through a phase separator and evaporated. The crude product was purified through preparative HPLC. (Kromasil C₈, 250×50 Idmm using an increasing gradient of MeCN with a second acid eluent (H₂O/MeCN/FA, 95/5/0.2)) to give 1-{5-Butyryl-3-cyano-6-[(2-oxopyrrolidin-1-yl)methyl]pyridin-2-yl}-N-[(1-phenylethyl)sulfonyl]piperidine-4-carboxamide as a white solid. Yield: 20 mg (9%)

MSm/z: 566 (M+1), 564 (M−1)

GTPγS(IC₅₀ μM):

### Example 69

### 1-{5-Butyryl-3-chloro-6-[(2-oxopyrrolidin-1-yl)methyl]pyridin-2-yl}-N-{[(4-fluorophenyl)(methyl)amino]sulfonyl}-N-methylpiperidine-4-carboxamide

A microwave vial was charged with methyliodide (180 μl; 2.90 mmol), 1-{5-Butyryl-3-chloro-6-[(2-oxopyrrolidin-1-yl)methyl]pyridin-2-yl}-N-{[(4-fluorophenyl)(methyl)amino]sulfonyl}piperidine-4-carboxamide (143 mg; 0.241 mmol), DIPEA (210 μl; 1.21 mmol) and DMF (4.0 mL) and the mixture was heated to 110° C. for 30 minutes in a microwave oven (single node heating). A white precipitate had formed. The mixture was partitioned between NH₄Cl and EtOAc, washed with water, the combined aqueous phase was back extracted with EtOAc and the combined organic phases were concentrated to give a crude product which was purified by preparative HPLC. (Kromasil C₈, 250×50 Idmm using an increasing gradient of MeCN with a second acid eluent (H₂O/MeCN/FA, 95/5/0.2)) to give 1-{5-butyryl-3-chloro-6-[(2-oxopyrrolidin-1-yl)methyl]pyridin-2-yl}-N-{[(4-fluorophenyl)(methyl)amino]sulfonyl}-N-methylpiperidine-4-carboxamide

As a white solid. Yield: 133 mg (91%).

¹H NMR (400 MHz, CDCl₃) δ 0.98 (3H, t, J=8.5 Hz), 1.64-1.87 (6H, m), 2.08 (2H, quintet, J=7.5 Hz), 2.47 (2H, t, J=8.3 Hz), 2.76-2.84 (4H, m), 2.88-2.97 (1H, m), 3.17 (3H, s), 3.41 (3H, s), 3.49 (2H, t, J=7.0 Hz), 4.09-4.17 (2H, m), 4.81 (2H, s), 7.08-7.15 (2H, m), 7.31-7.38 (2H, m), 7.93 (1H, s)

MSm/z: 608 (M+1), 606 (M−1)

GTPγS(IC₅₀ μM): 3.67

### Example 70

### 1-{5-Butyryl-3-cyano-6-[(2-oxopyrrolidin-1-yl)methyl]pyridin-2-yl}-N-[(cyclopentylmethyl)sulfonyl]-N-methylpiperidine-4-carboxamide

Prepared according to the procedure described in Example 69 from 1-{5-butyryl-3-cyano-6-[(2-oxopyrrolidin-1-yl)methyl]pyridin-2-yl}-N-[(cyclopentylmethyl)sulfonyl]piperidine-4-carboxamide (151 mg, 0.278 mmol) to give 1-{5-butyryl-3-cyano-6-[(2-oxopyrrolidin-1-yl)methyl]pyridin-2-yl}-N-[(cyclopentylmethyl)sulfonyl]-N-methylpiperidine-4-carboxamide as a white solid. Yield: 123 mg (79%).

¹H NMR (400 MHz, CDCl₃) δ 0.98 (3H, t, J=7.8 Hz), 1.23-1.38 (2H, m), 1.51-1.77 (6H, m), 1.78-2.02 (7H, m), 2.03-2.14 (2H, m), 2.28-2.38 (1H, m), 2.37-2.48 (2H, m), 2.73-2.84 (2H, m), 3.21-3.33 (5H, m), 3.34-3.42 (2H, m), 3.42-3.53 (2H, m), 4.58-4.72 (2H, m), 4.84 (2H, s), 8.19 (1H, s)

MSm/z: 558 (M+1), 556 (M−1)

GTPγS(IC₅₀ μM): 0.903

### Example 71

### 1-{3-chloro-6-[(2-oxopyrrolidin-1-yl)methyl]-5-pentanoylpyridin-2-yl}-N-[(2,4-difluorobenzyl)sulfonyl]-N-methylpiperidine-4-carboxamide

Prepared according to the procedure described in Example 69 from 1-{3-chloro-6-[(2-oxopyrrolidin-1-yl)methyl]-5-pentanoylpyridin-2-yl}-N-[(2,4-difluorobenzyl)sulfonyl]piperidine-4-carboxamide (22 mg, 0.04 mmol) to give 1-{3-chloro-6-[(2-oxopyrrolidin-1-yl)methyl]-5-pentanoylpyridin-2-yl}-N-[(2,4-difluorobenzyl)sulfonyl]-N-methylpiperidine-4-carboxamide as a white solid. Yield: 21 mg (95%).

MSm/z: 616 (M+1), 614 (M−1)

GTPγS(IC₅₀ μM): 0.903

### Example 72

### N-(benzylsulfonyl)-1-{5-butyryl-3-fluoro-6-[(2-oxopyrrolidin-1-yl)methyl]pyridin-2-yl}piperidine-4-carboxamide

### (a) 2,6-Dichloro-5-fluoronicotinoyl chloride

A suspension of 2,6-dichloro-5-fluoronicotinic acid (4.3 g, 20.5 mmol) in toluene (20 mL) and thionyl chloride (20 mL, 276 mmol) was refluxed under an N₂-atmosphere for 3 hours. The mixture was cooled and the solvent was concentrated in vacuo and the residue was co-evaporated twice with toluene to give 2,6-dichloro-5-fluoronicotinoyl chloride as a yellow oil which was used in the next step without further purification assuming quantitative yield of the product.

### (b) Ethyl 2,6-dichloro-5-fluoronicotinate

Cold ethanol (40 mL) was added to 2,6-dichloro-5-fluoronicotinoyl chloride (4.7 g, 20.5 mmol) at 0° C., the mixture was stirred for 15 minutes at 0° C. followed by 1 hour at reflux under an N₂-atmosphere. The EtOH was concentrated in vacuo and the residue was dissolved in EtOAc (130 mL) and the organic phase was washed with KHCO₃ (15 mL), water (15 mL), brine (15 mL), dried (MgSO₄), filtered and concentrated in vacuo to give ethyl 2,6-dichloro-5-fluoronicotinate as an oil. The crude product was used in the next step without further purification. Yield: 4.64 g (95%).

¹H NMR (400 MHz, CDCl₃) δ 1.42 (3H, t), 4.44 (2H, q), 8.00 (1H, d).

### (c) Ethyl 6-[4-(tert-butoxycarbonyl)piperidin-1-yl]-2-chloro-5-fluoronicotinate

Ethyl 2,6-dichloro-5-fluoronicotinate (2.1 g, 8.8 mmol) and tert-butyl piperidine-4-carboxylate (2.06 g, 11.1 mmol) were added to DIPEA (3.1 mL, 17.6 mmol) and EtOH (10 mL), the reaction mixture was heated to 100° C. for 30 minutes. EtOAc (150 mL) was added and the organic phase was washed with 1 M KHSO₄ (2×10 mL), NaHCO₃ (aq, sat) 10 mL, Brine (10 mL), dried (Na₂SO₄), filtered and concentrated in vacuo. The residue was purified by flash chromatography, pethroleum ether/EtOAc 9:1 to 7: as eluent, to give ethyl 6-[4-(tert-butoxycarbonyl)piperidin-1-yl]-2-chloro-5-fluoronicotinate as oil. Yield: 2.56 g (75%)

¹H NMR (400 MHz, CDCl₃) δ 1.38 (3H, t), 1.45 (9H, s), 1.71-1.82 (2H, m), 1.94-2.01 (2H, m), 2.44-2.54 (1H, m), 3.09-3.19 (2H, m), 4.28-4.37 (4H, m), 7.77 (1H, d).

### (d) Chloro[(2-oxopyrrolidin-1-yl)methyl]zinc

Prepared essentially to the procedure described by P. Knochel et al, JACS, 2007, pp. 5376-7 from 1-(chloromethyl)pyrrolidin-2-one to produce a 0.25 M THF solution of the reagent used in the next step.

### (e) Ethyl 6-[4-(tert-butoxycarbonyl)piperidin-1-yl]-5-fluoro-2-[(2-oxopyrrolidin-1-yl)methyl]nicotinate

A microwave vial was charged with chloro[(2-oxopyrrolidin-1-yl)methyl]zinc (2.2 mL of a 0.25 M solution in THF, 0.550 mmol), ethyl 6-[4-(tert-butoxycarbonyl)piperidin-1-yl]-2-chloro-5-fluoronicotinate (0.188 g, 0.486 mmol), bis(tri-tert-butylphosphine)palldium(0) (7 mg, 0.014 mmol) and N-methylpyrrolidine (2 mL) and heated in a microwave oven at 100° C. for 2×15 minutes. The crude product was purified by preparative HPLC (Kromasil C₈, using an increasing gradient of MeCN with an acidic second eluent (H₂O/MeCN/HOAc, 95/5/0.2)) to give ethyl 6-[4-(tert-butoxycarbonyl)piperidin-1-yl]-5-fluoro-2-[(2-oxopyrrolidin-1-yl)methyl]nicotinate as an colourless viscous oil. Yield: 110 mg (50%).

¹H NMR (400 MHz, CDCl₃) 1.36 (3H, t), 1.44 (9H, s), 1.67-1.78 (2H, m), 1.88-1.96 (2H, m), 2.06 (2H, m), 2.42-2.52 (3H, m), 3.10 (2H, m), 3.47 (2H, t), 4.24 (2H, m), 4.30 (2H, q), 4.85 (2H, s), 7.75 (1H, d).

MSm/z: 450 (M+1)

### (f) 6-[4-(tert-Butoxycarbonyl)piperidin-1-yl]-5-fluoro-2-[(2-oxopyrrolidin-1-yl)methyl]nicotinic acid

A mixture of ethyl 6-[4-(tert-butoxycarbonyl)piperidin-1-yl]-5-fluoro-2-[(2-oxopyrrolidin-1-yl)methyl]nicotinate (3.39 g, 7.54 mmol), LiOH (0.20 g, 8.30 mmol), EtOH (5 mL) and water (3 mL) was heated to reflux for 2 hours. An additional amount of LiOH (0.14 g, 5.8 mmol) was added and the heating was continued for 1 hour. Acetic acid (about 1 mL) was added and the mixture was concentrated. The crude product was purified by preparative HPLC (Kromasil C₈, using an increasing gradient of MeCN with an acidic second eluent (H₂O/MeCN/HOAc, 95/5/0.2)) to give 6-[4-(tert-butoxycarbonyl)piperidin-1-yl]-5-fluoro-2-[(2-oxopyrrolidin-1-yl)methyl]nicotinic acid. Yield: 2.10 g (66%).

¹H NMR (500 MHz, CDCl₃) δ 1.45 (9H, s), 1.70-1.80 (2H, m), 1.92-1.98 (2H, m), 2.11 (2H, m), 2.45-2.53 (3H, m), 3.11 (2H, m), 3.63 (2H, t), 4.26 (2H, m), 4.78 (2H, s), 7.72 (1H, d).

MSm/z: 422 (M+1), 420 (M−1)

### (g) tert-Butyl 1-{3-fluoro-5-(fluorocarbonyl)-6-[(2-oxopyrrolidin-1-yl)methyl]pyridin-2-yl}piperidine-4-carboxylate

Cyanuric fluoride (0.162 g, 1.2 mmol) was added to a mixture of 6-[4-(tert-butoxycarbonyl)piperidin-1-yl]-5-fluoro-2-[(2-oxopyrrolidin-1-yl)methyl]nicotinic acid (0.506 g, 1.2 mmol) and pyridine (0.114 g, 1.44 mmol) in DCM (6 mL) under an atmosphere of nitrogen and the mixture was stirred at r.t. over night. DIPEA (0.4 mL) was added and the mixture was extracted with DCM (3×10 mL). The combined organic phase was passed through a phase separator and evaporated to give tert-Butyl 1-{3-fluoro-5-(fluorocarbonyl)-6-[(2-oxopyrrolidin-1-yl)methyl]pyridin-2-yl}piperidine-4-carboxylate which was used without further purification in the next step.

### (h) di-tert-Butyl ({6-[4-(tert-butoxycarbonyl)piperidin-1-yl]-5-fluoro-2-[(2-oxopyrrolidin-1-yl)methyl]pyridin-3-yl}carbonyl)(ethyl)malonate

A solution of di-tert-butyl ethylmalonate (0.293 g, 1.2 mmol) in THF (5 mL) was added to sodium tert-pentoxide (0.145 g, 1.32 mmol) in THF (5 mL) at 0° C. The ice bath was removed and the reaction mixture was stirred 10 minutes at r.t. tert-Butyl 1-{3-fluoro-5-(fluorocarbonyl)-6-[(2-oxopyrrolidin-1-yl)methyl]pyridin-2-yl}piperidine-4-carboxylate (0.508 g, 1.2 mmol) in THF (2.5 mL) was added dropwise at 0° C. The ice bath was removed after 5 minutes and the reaction mixture was stirred 20 h at r.t. The crude product was purified by preparative HPLC (Kromasil C₈, using an increasing gradient of MeCN with an acidic second eluent (H₂O/MeCN/HOAc, 95/5/0.2)) to give di-tert-butyl ({6-[4-(tert-butoxycarbonyl)piperidin-1-yl]-5-fluoro-2-[(2-oxopyrrolidin-1-yl)methyl]pyridin-3-yl}carbonyl)(ethyl)malonate. Yield: 0.196 g (25%).

¹H NMR (400 MHz, CDCl₃) δ 1.05 (3H, t, J=7.4 Hz), 1.42 (18H, s), 1.45 (9H, s), 1.67-1.80 (2H, m), 1.90-1.98 (2H, m), 2.01-2.11 (2H, m), 2.22 (2H, q, J=7.4 Hz), 2.41-2.54 (3H, m), 3.08-3.18 (2H, m), 3.43 (2H, t, J=7.1 Hz), 4.24-4.32 (2H, m), 4.69 (2H, s), 7.67 (1H, d, J=15.0 Hz).

MSm/z: 648 (M+1).

### (i) 1-{5-Butyryl-3-fluoro-6-[(2-oxopyrrolidin-1-yl)methyl]pyridin-2-yl}piperidine-4-carboxylic acid

A mixture of TFA/DCM 1:1 (3 mL) was added to di-tert-butyl ({6-[4-(tert-butoxycarbonyl)piperidin-1-yl]-5-fluoro-2-[(2-oxopyrrolidin-1-yl)methyl]pyridin-3-yl}carbonyl)(ethyl)malonate (0.180 g, 0.278 mmol) and the reaction mixture was stirred 1.5 h at r.t. The reaction mixture was evaporated and the crude material dissolved in MeCN (1 mL). The reaction mixture was heated 5 minutes in a microwave oven, single node heating, at 100° C. The reaction mixture was concentrated in vacuo and co-evaporated with DCM (2×2 mL) to give 1-{5-butyryl-3-fluoro-6-[(2-oxopyrrolidin-1-yl)methyl]pyridin-2-yl}piperidine-4-carboxylic acid which was used in the next step without further purification. Yield: 0.100 g (92%).

¹H NMR (400 MHz, CDCl₃) δ 0.99 (3H, t, J=7.4 Hz), 1.64-1.75 (2H, m), 1.75-1.85 (2H, m), 1.96-2.04 (2H, m), 2.08-2.17 (2H, m), 2.56-2.67 (3H, m), 2.77 (2H, t, J=7.3 Hz), 3.12-3.22 (2H, m), 3.54 (2H, t, J=7.3 Hz), 4.23-4.32 (2H, m), 4.84 (2H, s), 7.63 (1H, d, J=14.5 Hz), 11.1 (1H, br s).

MSm/z: 392 (M+1)

### (j) N-(benzylsulfonyl)-1-{5-butyryl-3-fluoro-6-[(2-oxopyrrolidin-1-yl)methyl]pyridin-2-yl}piperidine-4-carboxamide

1-{5-butyryl-3-fluoro-6-[(2-oxopyrrolidin-1-yl)methyl]pyridin-2-yl}piperidine-4-carboxylic acid (0.134 g, 0.34 mmol) was slurried in DCM (3.5 mL). TBTU (0.132 g, 0.41 mmol) and then DIPEA (0.221 g, 0.299 mL, 1.71 mmol) were added. The mixture was stirred for 1 h at r.t. 1-Phenylmethanesulfonamide (0.070 g, 0.41 mmol) was added. The reaction mixture was stirred 17 h at r.t. The crude product was purified by preparative HPLC (Kromasil C₈, using an increasing gradient of MeCN with an acidic second eluent (H₂O/MeCN/HOAc, 95/5/0.2)) to give N-(benzylsulfonyl)-1-{5-butyryl-3-fluoro-6-[(2-oxopyrrolidin-1-yl)methyl]pyridin-2-yl}piperidine-4-carboxamide. Yield: 0.134 g (72%).

¹H NMR (400 MHz, CDCl₃) δ 0.97 (3H, t, J=7.4 Hz), 1.73-1.83 (6H, m), 1.93-2.04 (2H, m), 2.17-2.26 (2H, m), 2.48-2.58 (1H, m), 2.76 (2H, t, J=7.4 Hz), 2.88-2.98 (2H, m), 3.41 (2H, t, J=7.0 hz), 4.22-4.30 (2H, m), 4.58 (2H, s), 4.73 (2H, s), 7.30-7.37 (5H, m), 7.63 (1H, d, J=14.5 Hz), 10.60 (1H, br s).

MSm/z: 545 (M+1).

1. A compound of formula I or a pharmaceutically acceptable salt thereof: 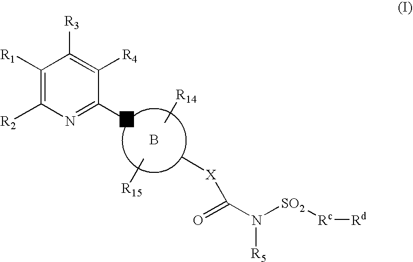
wherein
R₁ represents R₇C(O), R₁₇S, R₁₈C(S) or a group gII 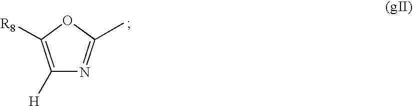
R₂ represents substituted (C₁-C₁₂)alkyl optionally interrupted by sulphur, substituted (C₁-C₁₂)alkoxy or substituted (C₁-C₁₂)alkylthio, wherein any one of these groups is substituted by one or more of azido, carboxy, cyano, (C₁-C₁₂)alkylcarbonyloxy, hydroxy(C₁-C₁₂)alkylcarbonyloxy, arylcarbonyloxy, heterocyclylcarbonyloxy, (C₁-C₁₂)alkyloxycarbonyl, (C₁-C₁₂)alkyl(C(S)), (C₁-C₁₂)alkyl(S(CO)), (C₁-C₁₂)alkylthio, hydroxy(C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl, heterocyclyl(C₁-C₁₂)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₁₂)alkyl, (C₁-C₁₂)alkylcarbonyl or R^{a(2)} and R^{b(2)} together with the nitrogen atom represent piperidine, pyrrolidine, azetidine or aziridine or any one of the groups 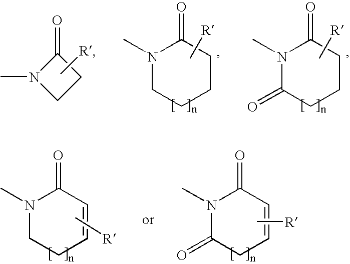
wherein n is an integer selected from 0, 1 and 2, and R′ is H, CN, OH, a halogen atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen atoms; or
R₂ represents substituted (C₁-C₁₂)alkoxy or substituted (C₁-C₁₂)alkylthio, wherein any one of these groups is substituted by one or more of any one of OH, aryl, (C₃-C₆)cycloalkyl or heterocyclyl; or
R₂ represents (C₁-C₁₂)alkylthio, substituted by one or more halogen atom(s); or
R₂ represents (C₁-C₁₂)alkylcarbonyloxy, aryl carbonyloxy, heterocyclylcarbonyloxy of which any one optionally is substituted by one or more of any one of the following groups or atoms: azido, cyano, halogen atom(s), OH, (C₁-C₁₂)alkoxy, (C₁-C₁₂)alkylthio, (C₁-C₁₂)alkylsulfinyl, (C₁-C₁₂)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₁₂)alkylthio, aryl(C₁-C₁₂)alkylsulfinyl, aryl(C₁-C₁₂)alkylsulfonyl, heterocyclyl(C₁-C₁₂)alkyl thio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, (C₁-C₁₂)alkylcarbonyl, arylcarbonyl, heterocyclylcarbonyl, aryl(C₁-C₁₂)alkylcarbonyl and heterocyclyl(C₁-C₁₂)alkylcarbonyl; or
R₂ represents unsubstituted (C₁-C₁₂)alkyl with the proviso that at the same time R₅ represents carboxy(C₁-C₁₂)alkyl; or
R₂ represents a group of formula ((R^{a(2)})N(R^{b(2)}))(CO)—, in which R^{a(2)} and R^{b(2)} each and independently represent H, (C₁-C₁₂)alkyl, aryl, aryl(C₁-C₁₂)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;
R₃ represents H, CN, NO₂, halogen, (C₁-C₁₂)alkyl optionally interrupted by oxygen and/or optionally substituted by OH, aryl, cycloalkyl, heterocyclyl or one or more halogen atoms; or
R₃ represents (C₁-C₁₂)alkoxy optionally substituted by one or more halogen atoms; or
R₃ represents (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;
R₄ represents H, CN, NO₂, halogen, (C₁-C₁₂)alkyl optionally interrupted by oxygen and/or optionally substituted by OH, COOH, (C₁-C₆)alkoxycarbonyl, aryl, cycloalkyl, heterocyclyl or one or more halogen atoms; or
R₄ represents (C₃-C₆)cycloalkyl, hydroxy(C₁-C₁₂)alkyl, (C₁-C₁₂)alkylC(O), (C₁-C₁₂)alkylcycloalkyl, (C₁-C₁₂)alkoxy wherein the alkoxy group may optionally be substituted by one or more halogen atoms, OH and/or COOH and/or (C₁-C₆)alkoxycarbonyl; or
R₄ represents (C₁-C₁₂)alkylthioC(O), (C₁-C₁₂)alkylC(S), (C₁-C₁₂)alkoxyC(O), (C₃-C₆)cycloalkoxy, aryl, aryl(C₁-C₁₂)alkoxy, aryl(C₁-C₁₂)alkyl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl, (C₃-C₆)cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;
R₅ represents H or (C₁-C₁₂)alkyl or carboxy(C₁-C₆)alkyl; with the proviso that when R₂ is unsubstituted (C₁-C₁₂)alkyl, R₅ represents carboxy(C₁-C₁₂)alkyl;
R₇ represents (C₁-C₁₂)alkyl optionally interrupted by oxygen, and/or optionally substituted by OH, aryl, cycloalkyl, heterocyclyl or one or more halogen atoms; or
R₇ represents (C₂-C₁₂)alkenyl optionally interrupted by oxygen, and/or optionally substituted by OH, aryl, cycloalkyl, heterocyclyl or one or more halogen atoms; or
R₇ represents (C₂-C₁₂)alkynyl optionally interrupted by oxygen, and/or optionally substituted by OH, aryl, cycloalkyl, heterocyclyl or one or more halogen atoms; or
R₇ represents (C₃-C₆)cycloalkyl, hydroxy(C₁-C₁₂)alkyl, aryl or heterocyclyl;
R₈ represents H, (C₁-C₁₂)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;
R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{c}; wherein R^{c} represents aryl, cycloalkyl, heterocyclyl or (C₁-C₁₂)alkyl optionally substituted by one or more of halogen atoms, OH, aryl, cycloalkyl and heterocyclyl; or
R₁₄ represents aryl, aryl(C₁-C₁₂)alkoxy, aryl(C₁-C₁₂)alkyl, (C₃-C₆)cycloalkyl(C₁-C₁₂)alkoxy, heterocyclyl, a halogen atom, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O), (C₁-C₁₂)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{c}; wherein R^{c} represents aryl, cycloalkyl, heterocyclyl or (C₁-C₁₂)alkyl optionally substituted by one or more of halogen atoms, OH, aryl, cycloalkyl and heterocyclyl; or
R₁₅ represents aryl, aryl(C₁-C₁₂)alkoxy, aryl(C₁-C₁₂)alkyl, (C₃-C₆)cycloalkyl(C₁-C₁₂)alkoxy, heterocyclyl, a halogen atom, (C₃-C₆)cycloalkyl, hydroxy(C₁-C₁₂)alkyl, (C₁-C₁₂)alkoxy, (C₃-C₆)cycloalkoxy, (C₁-C₁₂)alkylsulfinyl, (C₁-C₁₂)alkylsulfonyl, (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkoxy, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)alkyl, C₁-C₁₂)alkylC(O), (C₁-C₁₂)alkoxyC(O) or R^{a(15)} and R^{b(15)} together with the nitrogen atom represent piperidine, pyrrolidine, azetidine or aziridine;
R₁₇ represents (C₁-C₁₂)alkyl optionally interrupted by oxygen and/or optionally substituted by OH, aryl, cycloalkyl, heterocyclyl or one or more halogen atoms; or
R₁₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;
R₁₈ represents (C₁-C₁₂)alkyl optionally interrupted by oxygen and/or optionally substituted by OH, aryl, cycloalkyl, heterocyclyl or one or more halogen atoms; or
R₁₈ represents (C₃-C₆)cycloalkyl, hydroxy(C₁-C₁₂)alkyl, (C₁-C₁₂)alkoxy, (C₃-C₆)cycloalkoxy, aryl or heterocyclyl;
R^{c} is a single bond or represents an unsubstituted or monosubstituted or polysubstituted (C₁-C₄)alkylene group, (C₁-C₄)oxoalkylene group, (C₁-C₄)alkyleneoxy or oxy-(C₁-C₄)alkylene group, wherein any substituents each individually and independently are selected from (C₁-C₄)alkyl, (C₁-C₄)alkoxyl, oxy-(C₁-C₄)alkyl, (C₂-C₄)alkenyl, (C₂-C₄)alkynyl, (C₃-C₆)cycloalkyl, carboxyl, carboxy-(C₁-C₄)alkyl, aryl, heterocyclyl, nitro, cyano, halogeno, hydroxyl, NR^{a(Rc)}R^{b(Rc)} in which R^{a(Rc)} and R^{b(Rc)} individually and independently from each other represents hydrogen, (C₁-C₄)alkyl or R^{a(Rc)} and R^{b(Rc)} together with the nitrogen atom represent piperidine, pyrrolidine, azetidine or aziridine; or
R^{c} represents imino (—NH—), N-substituted imino (—NR₁₉—), (C₁-C₄)alkyleneimino or N-substituted (C₁-C₄)alkyleneimino (—N(R₁₉)—((C₁-C₄)alkylene) wherein the mentioned alkylene groups are unsubstituted or monosubstituted or polysubstituted with any substituents according to above;
R₁₉ represents H or (C₁-C₄)alkyl;
R^{d} represents (C₁-C₁₂)alkyl, (C₃-C₈)cycloalkyl, aryl or heterocyclyl, and any one of these groups optionally substituted with one or more halogen atoms and/or one or more of the following groups: OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, halogen substituted (C₁-C₁₂)alkylthio, (C₃-C₆)cycloalkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₁₂)alkyl, (C₁-C₁₂)alkylC(O) or R^{a(Rd)} and R^{b(Rd)} together with the nitrogen atom represent piperidine, pyrrolidine, azetidine or aziridine;
X represents a single bond, imino (—NH—), methylene (—CH₂—), iminomethylene (—CH₂—NH—) wherein the carbon is connected to the B-ring/ring system, methyleneimino (—NH—CH₂—) wherein the nitrogen is connected to the B-ring/ring system and any carbon and/or nitrogen in these groups may optionally be substituted with (C₁-C₆) alkyl; or
X represents a group (—CH₂—)ₙ wherein n=2-6, which optionally is unsaturated and/or substituted by one or more substituents selected from halogen, hydroxyl or (C₁-C₆)alkyl;
B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions, wherein the substituents R₁₄ and R₁₅ are connected to the B ring/ring system in such a way that no quarternary ammonium compounds are formed (by these connections).

2. A compound according to claim 1 wherein:
R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups 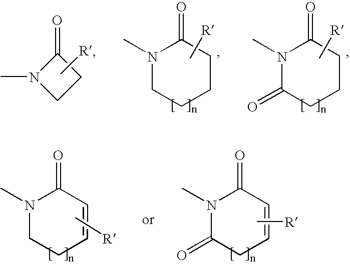
wherein n is an integer chosen from 0, 1 and 2, and R′ is H, CN, OH, a halogen atom, or one of the groups (C₁-C₈)alkyl, aryl, (C₁-C₈)alkoxy, (C₁-C₈)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen atoms; or
R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; or
R₂ represents (C₁-C₆)alkylthio, substituted by one or more halogen atom(s); or
R₂ represents (C₁-C₆)alkylcarbonyloxy, aryl carbonyloxy, heterocyclylcarbonyloxy of which any one optionally is substituted by one or more of any one of the following groups or atoms: azido, cyano, halogen atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; or
R₂ represents unsubstituted (C₁-C₆)alkyl with the proviso that at the same time R₅ represents carboxy(C₁-C₆)alkyl; or
R₂ represents a group of formula ((R^{a(2)})N(R^{b(2)}))(CO)—, in which R^{a(2)} and R^{b(2)} each and independently represent H, (C₁-C₆)alkyl, aryl, aryl(C₁-C₆)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;
R₃ represents H, CN, NO₂, halogen, (C₁-C₆)alkyl optionally interrupted by oxygen and/or optionally substituted by OH, aryl, cycloalkyl, heterocyclyl or one or more halogen atoms; or
R₃ represents (C₁-C₆)alkoxy optionally substituted by one or more halogen atoms; or
R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a (3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl optionally interrupted by oxygen and/or optionally substituted by OH, COOH, (C₁-C₆)alkoxycarbonyl, aryl, cycloalkyl, heterocyclyl or one or more halogen atoms; or
R₄ represents (C₃-C₆)cycloalkyl, hydroxy(C₁-C₆)alkyl, (C₁-C₆)alkylC(O), (C₁-C₆)alkylcycloalkyl, (C₁-C₆)alkoxy wherein the alkoxy group may optionally be substituted by one or more halogen atoms, OH and/or COOH and/or (C₁-C₆)alkoxycarbonyl; or
R₄ represents (C₁-C₆)alkylthioC(O), (C₁-C₆)alkylC(S), (C₁-C₆)alkoxyC(O), (C₃-C₆)cycloalkoxy, aryl, aryl(C₁-C₆)alkoxy, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl, (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₅ represents H or (C₁-C₆)alkyl or carboxy(C₁-C₆)alkyl; with the proviso that when R₂ is unsubstituted (C₁-C₆)alkyl, R₅ represents carboxy(C₁-C₆)alkyl;
R₇ represents (C₁-C₆)alkyl optionally interrupted by oxygen, and/or optionally substituted by OH, aryl, cycloalkyl, heterocyclyl or one or more halogen atoms; or
R₇ represents (C₂-C₆)alkenyl optionally interrupted by oxygen, and/or optionally substituted by OH, aryl, cycloalkyl, heterocyclyl or one or more halogen atoms; or
R₇ represents (C₂-C₆)alkynyl optionally interrupted by oxygen, and/or optionally substituted by OH, aryl, cycloalkyl, heterocyclyl or one or more halogen atoms; or
R₇ represents (C₃-C₆)cycloalkyl, hydroxy(C₁-C₆)alkyl, aryl or heterocyclyl;
R₈ represents H, (C₁-C₆)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen (atoms, OH, aryl, cycloalkyl and heterocyclyl; or
R₁₄ represents aryl, aryl(C₁-C₆)alkoxy, aryl(C₁-C₆)alkyl, (C₃-C₆)cycloalkyl(C₁-C₆)alkoxy, heterocyclyl, a halogen atom, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl or (C₃-C₆)cycloalkyl(C₁-C₆)alkylsulfonyl, a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₅ represents aryl, aryl(C₁-C₆)alkoxy, aryl(C₁-C₆)alkyl, (C₃-C₆)cycloalkyl(C₁-C₆)alkoxy, heterocyclyl, a halogen atom, (C₃-C₆)cycloalkyl, hydroxy(C₁-C₆)alkyl, (C₁-C₆)alkoxy, (C₃-C₆)cycloalkoxy, (C₁-C₆)alkylsulfinyl, (C₁-C₆)alkylsulfonyl, (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkoxy, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O), (C₁-C₆)alkoxyC(O) or R^{a(15)} and R^{b(15)} together with the nitrogen atom represent piperidine, pyrrolidine, azetidine or aziridine;
R₁₇ represents (C₁-C₆)alkyl optionally interrupted by oxygen and/or optionally substituted by OH, aryl, cycloalkyl, heterocyclyl or one or more halogen atoms; or
R₁₇ represents (C₃-C₆)cycloalkyl, hydroxy(C₁-C₆)alkyl, (C₁-C₆)alkoxy, (C₃-C₆)cycloalkoxy, aryl or heterocyclyl;
R₁₈ represents (C₁-C₆)alkyl optionally interrupted by oxygen and/or optionally substituted by OH, aryl, cycloalkyl, heterocyclyl or one or more halogen atoms; or
R₁₈ represents (C₃-C₆)cycloalkyl, hydroxy(C₁-C₆)alkyl, (C₁-C₆)alkoxy, (C₃-C₆)cycloalkoxy, aryl or heterocyclyl; and
R^{d} represents (C₁-C₆)alkyl, (C₃-C₈)cycloalkyl, aryl or heterocyclyl, and a one of these groups optionally substituted with one or more halogen atoms and/or one or more of the following groups: OH, CN, NO₂, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, halogen substituted (C₁-C₆)alkylthio, (C₃-C₆)cycloalkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, tri(C₁-C₄)alkylsilyl or a group of formula NR^{a(Rd)}R^{b(Rd)} in which R^{a(Rd)} and R^{b(Rd)} independently represent H, (C₁-C₆)alkyl, (C₁-C₆)alkylC(O) or R^{a(Rd)} and R^{b(Rd)} together with the nitrogen atom represent piperidine, pyrrolidine, azetidine or aziridine.

3. A compound according to claim 2 wherein:
R₁ represents R₇C(O), or a group gII 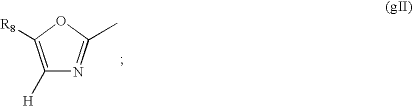
R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups 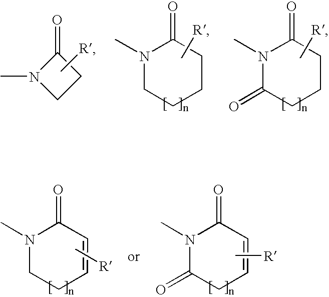
wherein n is an integer chosen from 0, 1 and 2, and R′ is H, CN, OH, a halogen atom, or one of the groups (C₁-C₆)alkyl, aryl, (C₁-C₆)alkoxy, (C₁-C₆)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen atoms; or
R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; or
R₂ represents (C₁-C₆)alkylthio, substituted by one or more halogen atom(s); or
R₂ represents (C₁-C₆)alkylcarbonyloxy, aryl carbonyloxy, heterocyclylcarbonyloxy of which any one optionally is substituted by one or more of any one of the following groups or atoms: azido, cyano, halogen atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; or
R₂ represents unsubstituted (C₁-C₆)alkyl with the proviso that at the same time R₅ represents carboxy(C₁-C₆)alkyl; or
R₂ represents a group of formula ((R^{a(2)})N(R^{b(2)}(CO)—, in which R^{a(2)} and R^{b(2)} each and independently represent H, (C₁-C₆)alkyl, aryl, aryl(C₁-C₆)alkyl, heterocyclyl(C₃-C₆)cycloalkyl, (C₃-C₆)cycloalkyl(C₁-C₆)alkyl, heterocyclyl(C₁-C₆)alkyl or R^{a(2)} and R^{b(2)} together with the nitrogen atom represent piperidine, pyrrolidine, azetidine or aziridine;
R₃ represents H, CN, NO₂, halogen, (C₁-C₆)alkyl optionally interrupted by oxygen and/or optionally substituted by OH, aryl, cycloalkyl, heterocyclyl or one or more halogen atoms; or
R₃ represents (C₁-C₆)alkoxy optionally substituted by one or more halogen atoms; or
R₃ represents (C₃-C₆)cycloalkyl, hydroxy(C₁-C₆)alkyl, (C₁-C₆)alkylC(O), (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a (3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl optionally interrupted by oxygen and/or optionally substituted by OH, COOH, aryl, cycloalkyl, heterocyclyl or one or more halogen atoms; or
R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by one or more halogen atoms, OH and/or COOH and/or methoxycarbonyl; or
R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₈ represents H, (C₁-C₆)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₆)alkyl, (C₁-C₆)alkoxy, (C₃-C₆)cycloalkoxy, aryl or heterocyclyl;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₄ represents aryl, aryl(C₁-C₆)alkoxy, aryl(C₁-C₆)alkyl, (C₃-C₆)cycloalkyl(C₁-C₆)alkoxy, heterocyclyl, a halogen atom, (C₃-C₆)cycloalkyl, hydroxy(C₁-C₆)alkyl, (C₁-C₆)alkoxy, (C₃-C₆)cycloalkoxy, or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine; and
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₅ represents aryl, aryl(C₁-C₆)alkoxy, aryl(C₁-C₆)alkyl, (C₃-C₆)cycloalkyl(C₁-C₆)alkoxy, heterocyclyl, a halogen atom, (C₃-C₆)cycloalkyl, hydroxy(C₁-C₆)alkyl, (C₁-C₆)alkoxy, (C₃-C₆)cycloalkoxy, or a group of formula NR^{a(15)}R^{b(15)} in which R^{a}(15) and R^{b(15)} independently represent H, (C₁-C₆)alkyl, (C₁-C₆)alkylC(O), (C₁-C₆)alkoxyC(O) or R^{a}(15) and R^{b(15)} together with the nitrogen atom represent piperidine, pyrrolidine, azetidine or aziridine.

4. A compound according to claim 3 wherein:
R₂ represents substituted (C₁-C₆)alkyl optionally interrupted by sulphur, substituted (C₁-C₆)alkoxy or substituted (C₁-C₆)alkylthio, wherein any one of these groups is substituted by one or more of azido, carboxy, cyano, (C₁-C₆)alkylcarbonyloxy, hydroxy(C₁-C₆)alkylcarbonyloxy, arylcarbonyloxy, heterocyclylcarbonyloxy, (C₁-C₆)alkyloxycarbonyl, (C₁-C₆)alkyl(C(S)), (C₁-C₆)alkyl(S(CO)), (C₁-C₆)alkylthio, hydroxy(C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl, heterocyclyl(C₁-C₆)alkylcarbonyl or of a group of formula NR^{a(2)}R^{b(2)} or —(CO)NR^{a(2)}R^{b(2)}, in which R^{a(2)} and R^{b(2)} each and independently represent H, (C₁-C₆)alkyl, (C₁-C₆)alkylcarbonyl or R^{a(2)} and R^{b(2)} together with the nitrogen atom represent piperidine, pyrrolidine, azetidine or aziridine or any one of the groups 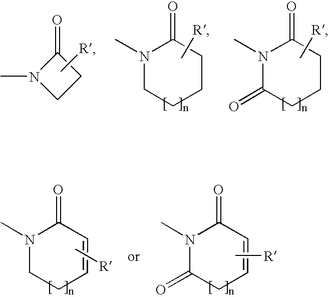
wherein n is an integer chosen from 0, 1 and 2, and R′ is H, CN, OH, a halogen atom, or one of the groups (C₁-C₄)alkyl, aryl, (C₁-C₄)alkoxy, (C₁-C₄)alkylthio, (C₁-C₇)cycloalkyl, heterocyclyl, aryl(C₁-C₆)alkyl, (C₁-C₇)cycloalkyl(C₁-C₆)alkyl, heterocyclyl(C₁-C₆)alkyl, of which groups any one optionally is substituted by one or more OH and/or one or more halogen atoms; or
R₂ represents substituted (C₁-C₆)alkoxy or substituted (C₁-C₆)alkylthio, wherein any one of these groups is substituted by one or more of any one of OH, aryl, (C₃-C₆)cycloalkyl or heterocyclyl; or
R₂ represents (C₁-C₆)alkylthio, substituted by one or more halogen atom(s); or
R₂ represents (C₁-C₆)alkylcarbonyloxy, aryl carbonyloxy, heterocyclylcarbonyloxy of which any one optionally is substituted by one or more of any one of the following groups or atoms: azido, cyano, halogen atom(s), OH, (C₁-C₆)alkoxy, (C₁-C₆)alkylthio, (C₁-C₆)alkylsulfinyl, (C₁-C₆)alkylsulfonyl, (C₃-C₆)cycloalkyloxy, (C₃-C₆)cycloalkylthio, (C₃-C₆)cycloalkylsulfinyl, (C₃-C₆)cycloalkylsulfonyl, aryloxy, arylthio, arylsulfinyl, arylsulfonyl, heterocyclyloxy, heterocyclylthio, heterocyclylsulfinyl, heterocyclylsulfonyl, aryl(C₁-C₆)alkylthio, aryl(C₁-C₆)alkylsulfinyl, aryl(C₁-C₆)alkylsulfonyl, heterocyclyl(C₁-C₆)alkyl thio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl, (C₁-C₆)alkylcarbonyl, arylcarbonyl, heterocyclylcarbonyl, aryl(C₁-C₆)alkylcarbonyl and heterocyclyl(C₁-C₆)alkylcarbonyl; or
R₂ represents unsubstituted (C₁-C₆)alkyl with the proviso that at the same time R₅ represents carboxy(C₁-C₆)alkyl;
R₃ represents H or a group of formula NR^{a (3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents CN, halogen, further R₄ represents (C₁-C₆)alkylC(O), (C₁-C₆)alkoxy wherein the alkoxy group may optionally be substituted by one or more halogen atoms, OH and/or COOH and/or methoxycarbonyl;
R₇ represents (C₁-C₆)alkyl optionally interrupted by oxygen, and/or optionally substituted by OH, aryl, cycloalkyl, heterocyclyl or one or more halogen atoms; or
R₇ represents (C₂-C₆)alkenyl optionally interrupted by oxygen, and/or optionally substituted by OH, aryl, cycloalkyl, heterocyclyl or one or more halogen atoms; or
R₇ represents (C₂-C₆)alkynyl optionally interrupted by oxygen, and/or optionally substituted by OH, aryl, cycloalkyl, heterocyclyl or one or more halogen atoms; or
R₇ represents (C₃-C₆)cycloalkyl or hydroxy(C₁-C₆)alkyl;
R₈ represents H, (C₁-C₆)alkyl optionally interrupted by oxygen, and/or optionally substituted by aryl, cycloalkyl, heterocyclyl or one or more halogen atoms;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{e}; wherein R^{e} represents aryl, cycloalkyl, heterocyclyl or (C₁-C₆)alkyl optionally substituted by one or more of halogen atoms, OH, aryl, cycloalkyl and heterocyclyl; or
R₁₄ represents or a group of formula NR^{a(14)}R^{b(14)} in which R^{a(14)} and R^{b(14)} independently represent H, (C₁-C₆)alkyl, (C₁-C₆)alkylC(O), (C₁-C₆)alkoxyC(O) or R^{a(14)} and R^{b(14)} together with the nitrogen atom represent piperidine, pyrrolidine, azetidine or aziridine; and
R₁₅ represents H.

5. A compound according to claim 1 wherein:
R₁ is selected from methylcarbonyl, ethylcarbonyl, n-propylcarbonyl, isopropylcarbonyl, cyclopropylcarbonyl, n-butylcarbonyl, 4-buten-1-ylcarbonyl, 3,3,3-trifluoropropylcarbonyl and 5-ethyl-1,3-oxazol-2-yl;
R₂ is selected from (2-oxopyrrolidin-1-yl)methyl and (2-oxopiperidin-1-yl)methyl;
R₃ is H;
R₄ is selected from fluoro, chloro and cyano;
R₅ is H or methyl;
R₇ is selected from methyl, ethyl, n-propyl, isopropyl, cyclopropyl, n-butyl, 4-buten-1-yl and 3,3,3-trifluoropropyl;
R₈ is ethyl;
R₁₄ is H;
R₁₅ is H;
R^{c} is selected from methylene (—CH₂—), methylmethine (—CH(CH₃)—), imino (—NH—) and methylimino (—N(CH₃)—);
R₁₉ is chosen from H or methyl;
R^{d} is selected from cyclobutyl, cyclopentyl, cyclohexyl, phenyl, 4-methylphenyl, 4-isopropylphenyl, 4-methoxyphenyl, 4-fluorophenyl, 4-chlorophenyl, 2,4-difluorophenyl, and 4-cyanophenyl;
X represents a single bond; and
B is 4-piperidin-1-ylene and the substituents R₁₄ and R₁₅ are connected to the B ring/ring system, in such a way that no quarternary ammonium compounds are formed (by these connections).

6. A compound according to claim 1 which is of the formula (Ia):

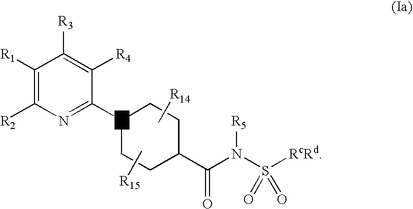

7. A compound according to claim 1 which is of the formula (Ib):

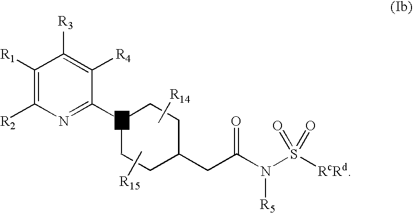

8. A compound according to claim 1 which is of the formula (Ic):

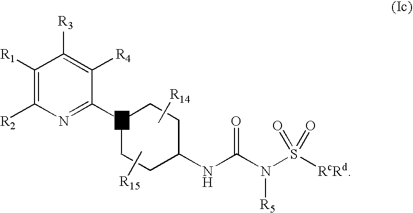

9. A compound according to claim 1 which is of the formula (Id):

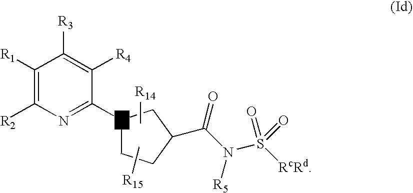

10. A compound according to claim 1 which is of the formula (Ie):

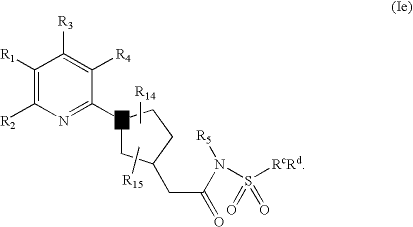

11. A compound according to claim 1 which is of the formula (If):

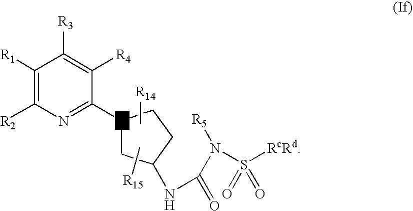

12. A compound according to claim 1 which is of the formula (Ig):

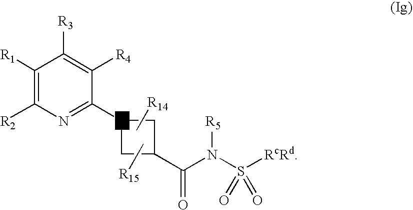

13. A compound according to claim 1 which is of the formula (Ih):

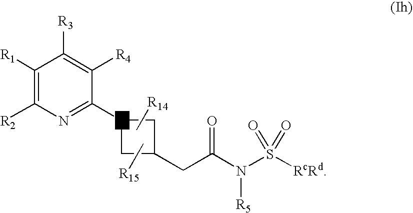

14. A compound according to claim 1 which is of the formula (Ii):

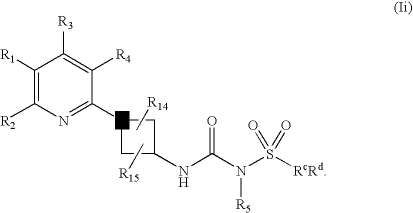

15. A compound according to claim 1 wherein
R₁ represents R₇C(O).

16. A compound according to claim 1 wherein R₁ represents a group gII

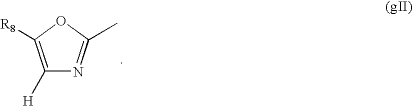

17. A compound according to claim 15 which is of the formula (Iaa):

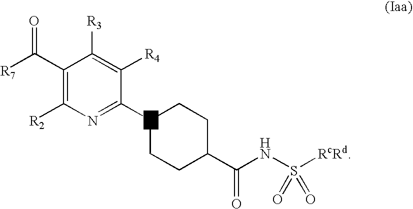

18. A compound according to claim 16 which is of the formula (Iab):

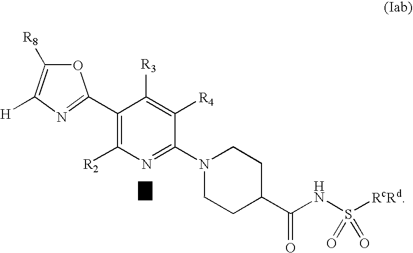

19. A compound selected from:
1-{5-acetyl-3-cyano-6-[(2-oxopyrrolidin-1-yl)methyl]pyridin-2-yl}-N-(benzylsulfonyl)piperidine-4-carboxamide
N-(benzylsulfonyl)-1-{5-butyryl-3-cyano-6-[(2-oxopyrrolidin-1-yl)methyl]pyridin-2-yl}piperidine-4-carboxamide
1-{5-acetyl-3-cyano-6-[(2-oxopiperidin-1-yl)methyl]pyridin-2-yl}-N-(benzylsulfonyl)piperidine-4-carboxamide
1-{5-butyryl-3-cyano-6-[(2-oxopiperidin-1-yl)methyl]pyridin-2-yl}-N-[(4-methylbenzyl)sulfonyl]piperidine-4-carboxamide
1-{5-butyryl-3-cyano-6-[(2-oxopyrrolidin-1-yl)methyl]pyridin-2-yl}-N-[(4-methylbenzyl)sulfonyl]piperidine-4-carboxamide
1-{5-butyryl-3-cyano-6-[(2-oxopyrrolidin-1-yl)methyl]pyridin-2-yl}-N-[(2,4-difluorobenzyl)sulfonyl]piperidine-4-carboxamide
1-{5-butyryl-3-cyano-6-[(2-oxopyrrolidin-1-yl)methyl]pyridin-2-yl}-N-[(cyclopentylmethyl)sulfonyl]piperidine-4-carboxamide
N-(benzylsulfonyl)-1-{3-cyano-5-(cyclopropylcarbonyl)-6-[(2-oxopyrrolidin-1-yl)methyl]pyridin-2-yl}piperidine-4-carboxamide
1-{3-cyano-5-(cyclopropylcarbonyl)-6-[(2-oxopyrrolidin-1-yl)methyl]pyridin-2-yl}-N-[(cyclopentylmethyl)sulfonyl]piperidine-4-carboxamide
N-(benzylsulfonyl)-1-{3-cyano-6-[(2-oxopyrrolidin-1-yl)methyl]-5-propionylpyridin-2-yl}piperidine-4-carboxamide
1-{3-cyano-6-[(2-oxopyrrolidin-1-yl)methyl]-5-propionylpyridin-2-yl}-N-[(4-methylbenzyl)sulfonyl]piperidine-4-carboxamide
1-{3-cyano-6-[(2-oxopyrrolidin-1-yl)methyl]-5-propionylpyridin-2-yl}-N-[(cyclopentylmethyl)sulfonyl]piperidine-4-carboxamide
1-{3-cyano-5-(cyclopropylcarbonyl)-6-[(2-oxopiperidin-1-yl)methyl]pyridin-2-yl}-N-{[methyl(phenyl)amino]sulfonyl}piperidine-4-carboxamide
1-{3-cyano-5-(cyclopropylcarbonyl)-6-[(2-oxopiperidin-1-yl)methyl]pyridin-2-yl}-N-[(cyclopentylmethyl)sulfonyl]piperidine-4-carboxamide
N-(benzylsulfonyl)-1-{3-cyano-5-isobutyryl-6-[(2-oxopyrrolidin-1-yl)methyl]pyridin-2-yl}piperidine-4-carboxamide
1-{3-cyano-5-isobutyryl-6-[(2-oxopyrrolidin-1-yl)methyl]pyridin-2-yl}-N-[(cyclopentylmethyl)sulfonyl]piperidine-4-carboxamide
1-{3-cyano-6-[(2-oxopyrrolidin-1-yl)methyl]-5-pentanoylpyridin-2-yl}-N-[(cyclopentylmethyl)sulfonyl]piperidine-4-carboxamide
N-(benzylsulfonyl)-1-{3-cyano-6-[(2-oxopyrrolidin-1-yl)methyl]-5-pentanoylpyridin-2-yl}piperidine-4-carboxamide
1-{3-cyano-6-[(2-oxopyrrolidin-1-yl)methyl]-5-pentanoylpyridin-2-yl}-N-[(4-methylbenzyl)sulfonyl]piperidine-4-carboxamide
1-{3-cyano-6-[(2-oxopyrrolidin-1-yl)methyl]-5-pentanoylpyridin-2-yl}-N-[(2,4-difluorobenzyl)sulfonyl]piperidine-4-carboxamide
1-{3-cyano-6-[(2-oxopyrrolidin-1-yl)methyl]-5-pentanoylpyridin-2-yl}-N-{[(4-fluorophenyl)(methyl)amino]sulfonyl}piperidine-4-carboxamide
1-{3-cyano-6-[(2-oxopyrrolidin-1-yl)methyl]-5-pentanoylpyridin-2-yl}-N-{[methyl(phenyl)amino]sulfonyl}piperidine-4-carboxamide
1-{3-cyano-5-(cyclopropylcarbonyl)-6-[(2-oxopyrrolidin-1-yl)methyl]pyridin-2-yl}-N-{[methyl(phenyl)amino]sulfonyl}piperidine-4-carboxamide
1-{3-cyano-5-(cyclopropylcarbonyl)-6-[(2-oxopyrrolidin-1-yl)methyl]pyridin-2-yl}-N-[(cyclobutylmethyl)sulfonyl]piperidine-4-carboxamide
1-{3-cyano-5-(cyclopropylcarbonyl)-6-[(2-oxopyrrolidin-1-yl)methyl]pyridin-2-yl}-N-{[(4-fluorophenyl)(methyl)amino]sulfonyl}piperidine-4-carboxamide
N-(benzylsulfonyl)-1-{3-cyano-6-[(2-oxopyrrolidin-1-yl)methyl]-5-pent-4-enoylpyridin-2-yl}piperidine-4-carboxamide
1-{3-cyano-6-[(2-oxopyrrolidin-1-yl)methyl]-5-pent-4-enoylpyridin-2-yl}-N-[(cyclopentylmethyl)sulfonyl]piperidine-4-carboxamide
1-{3-cyano-6-[(2-oxopyrrolidin-1-yl)methyl]-5-pent-4-enoylpyridin-2-yl}-N-{[(4-fluorophenyl)(methyl)amino]sulfonyl}piperidine-4-carboxamide
1-{3-cyano-6-[(2-oxopyrrolidin-1-yl)methyl]-5-pent-4-enoylpyridin-2-yl}-N-{[methyl(phenyl)amino]sulfonyl}piperidine-4-carboxamide
N-(benzylsulfonyl)-1-{5-butyryl-3-chloro-6-[(2-oxopyrrolidin-1-yl)methyl]pyridin-2-yl}piperidine-4-carboxamide
1-{5-butyryl-3-chloro-6-[(2-oxopyrrolidin-1-yl)methyl]pyridin-2-yl}-N-{[(4-fluorophenyl)(methyl)amino]sulfonyl}piperidine-4-carboxamide
1-{5-butyryl-3-chloro-6-[(2-oxopyrrolidin-1-yl)methyl]pyridin-2-yl}-N-[(4-methylbenzyl)sulfonyl]piperidine-4-carboxamide
1-{5-butyryl-3-cyano-6-[(2-oxopyrrolidin-1-yl)methyl]pyridin-2-yl}-N-{[(4-fluorophenyl)(methyl)amino]sulfonyl}piperidine-4-carboxamide
1-{5-butyryl-3-cyano-6-[(2-oxopyrrolidin-1-yl)methyl]pyridin-2-yl}-N-{[methyl(phenyl)amino]sulfonyl}piperidine-4-carboxamide
1-{5-butyryl-3-cyano-6-[(2-oxopyrrolidin-1-yl)methyl]pyridin-2-yl}-N-[(4-methoxybenzyl)sulfonyl]piperidine-4-carboxamide
1-{5-butyryl-3-cyano-6-[(2-oxopyrrolidin-1-yl)methyl]pyridin-2-yl}-N-[(cyclohexylmethyl)sulfonyl]piperidine-4-carboxamide
1-{5-butyryl-3-cyano-6-[(2-oxopyrrolidin-1-yl)methyl]pyridin-2-yl}-N-{[(4-fluorophenyl)amino]sulfonyl}piperidine-4-carboxamide
N-(anilinosulfonyl)-1-{5-butyryl-3-cyano-6-[(2-oxopyrrolidin-1-yl)methyl]pyridin-2-yl}piperidine-4-carboxamide
1-{5-butyryl-3-chloro-6-[(2-oxopyrrolidin-1-yl)methyl]pyridin-2-yl}-N-[(cyclopentylmethyl)sulfonyl]piperidine-4-carboxamide
1-{3-cyano-6-[(2-oxopyrrolidin-1-yl)methyl]-5-pent-4-enoylpyridin-2-yl}-N-[(4-methylbenzyl)sulfonyl]piperidine-4-carboxamide
1-{3-cyano-6-[(2-oxopyrrolidin-1-yl)methyl]-5-pent-4-enoylpyridin-2-yl}-N-[(2,4-difluorobenzyl)sulfonyl]piperidine-4-carboxamide
1-{3-cyano-6-[(2-oxopyrrolidin-1-yl)methyl]-5-(4,4,4-trifluorobutanoyl)pyridin-2-yl}-N-[(2,4-difluorobenzyl)sulfonyl]piperidine-4-carboxamide
1-{3-cyano-6-[(2-oxopyrrolidin-1-yl)methyl]-5-(4,4,4-trifluorobutanoyl)pyridin-2-yl}-N-[(4-methylbenzyl)sulfonyl]piperidine-4-carboxamide
1-{3-cyano-6-[(2-oxopyrrolidin-1-yl)methyl]-5-(4,4,4-trifluorobutanoyl)pyridin-2-yl}-N-[(cyclopentylmethyl)sulfonyl]piperidine-4-carboxamide
1-{3-cyano-6-[(2-oxopyrrolidin-1-yl)methyl]-5-(4,4,4-trifluorobutanoyl)pyridin-2-yl}-N-{[(4-fluorophenyl)(methyl)amino]sulfonyl}piperidine-4-carboxamide
1-{3-cyano-6-[(2-oxopyrrolidin-1-yl)methyl]-5-pentanoylpyridin-2-yl}-N-{[(4-fluorophenyl)amino]sulfonyl}piperidine-4-carboxamide
N-[(4-cyanobenzyl)sulfonyl]-1-{3-cyano-6-[(2-oxopyrrolidin-1-yl)methyl]-5-pentanoylpyridin-2-yl}piperidine-4-carboxamide
1-{3-cyano-6-[(2-oxopyrrolidin-1-yl)methyl]-5-pentanoylpyridin-2-yl}-N-[(cyclohexylmethyl)sulfonyl]piperidine-4-carboxamide
1-{3-cyano-6-[(2-oxopyrrolidin-1-yl)methyl]-5-pentanoylpyridin-2-yl}-N-[(4-isopropylbenzyl)sulfonyl]piperidine-4-carboxamide
1-{3-chloro-6-[(2-oxopyrrolidin-1-yl)methyl]-5-pent-4-enoylpyridin-2-yl}-N-{[(4-fluorophenyl)(methyl)amino]sulfonyl}piperidine-4-carboxamide
N-(benzylsulfonyl)-1-{3-chloro-6-[(2-oxopyrrolidin-1-yl)methyl]-5-pent-4-enoylpyridin-2-yl}piperidine-4-carboxamide
1-{5-butyryl-3-chloro-6-[(2-oxopyrrolidin-1-yl)methyl]pyridin-2-yl}-N-[(cyclohexylmethyl)sulfonyl]piperidine-4-carboxamide
1-{5-butyryl-3-chloro-6-[(2-oxopyrrolidin-1-yl)methyl]pyridin-2-yl}-N-[(2,4-difluorobenzyl)sulfonyl]piperidine-4-carboxamide
1-{3-chloro-6-[(2-oxopyrrolidin-1-yl)methyl]-5-pentanoylpyridin-2-yl}-N-[(cyclopentylmethyl)sulfonyl]piperidine-4-carboxamide
1-{3-chloro-6-[(2-oxopyrrolidin-1-yl)methyl]-5-pentanoylpyridin-2-yl}-N-[(2,4-difluorobenzyl)sulfonyl]piperidine-4-carboxamide
1-{3-chloro-6-[(2-oxopyrrolidin-1-yl)methyl]-5-pentanoylpyridin-2-yl}-N-{[(4-fluorophenyl)(methyl)amino]sulfonyl}piperidine-4-carboxamide
N-(benzylsulfonyl)-1-{3-chloro-6-[(2-oxopyrrolidin-1-yl)methyl]-5-pentanoylpyridin-2-yl}piperidine-4-carboxamide
N-(benzylsulfonyl)-1-{3-cyano-6-[(2-oxopyrrolidin-1-yl)methyl]-5-(4,4,4-trifluorobutanoyl)pyridin-2-yl}piperidine-4-carboxamide
N-[(4-chlorobenzyl)sulfonyl]-1-{3-chloro-6-[(2-oxopyrrolidin-1-yl)methyl]-5-pentanoylpyridin-2-yl}piperidine-4-carboxamide
1-{3-chloro-6-[(2-oxopyrrolidin-1-yl)methyl]-5-pentanoylpyridin-2-yl}-N-[(1-phenylethyl)sulfonyl]piperidine-4-carboxamide
1-{3-cyano-6-[(2-oxopyrrolidin-1-yl)methyl]-5-pentanoylpyridin-2-yl}-N-[(1-phenylethyl)sulfonyl]piperidine-4-carboxamide
1-{3-cyano-5-(5-ethyl-1,3-oxazol-2-yl)-6-[(2-oxopyrrolidin-1-yl)methyl]pyridin-2-yl}-N-{[(4-fluorophenyl)(methyl)amino]sulfonyl}piperidine-4-carboxamide
N-(benzylsulfonyl)-1-{3-cyano-5-(5-ethyl-1,3-oxazol-2-yl)-6-[(2-oxopyrrolidin-1-yl)methyl]pyridin-2-yl}piperidine-4-carboxamide
1-{3-chloro-5-(5-ethyl-1,3-oxazol-2-yl)-6-[(2-oxopyrrolidin-1-yl)methyl]pyridin-2-yl}-N-{[(4-fluorophenyl)(methyl)amino]sulfonyl}piperidine-4-carboxamide
N-(benzylsulfonyl)-1-{3-chloro-5-(5-ethyl-1,3-oxazol-2-yl)-6-[(2-oxopyrrolidin-1-yl)methyl]pyridin-2-yl}piperidine-4-carboxamide
N-(benzylsulfonyl)-1-{5-butyryl-3-chloro-6-[(2-oxopiperidin-1-yl)methyl]pyridin-2-yl}piperidine-4-carboxamide
1-{5-butyryl-3-chloro-6-[(2-oxopiperidin-1-yl)methyl]pyridin-2-yl}-N-{[(4-fluorophenyl)(methyl)amino]sulfonyl}piperidine-4-carboxamide
1-{5-butyryl-3-cyano-6-[(2-oxopyrrolidin-1-yl)methyl]pyridin-2-yl}-N-[(1-phenylethyl)sulfonyl]piperidine-4-carboxamide
1-{5-butyryl-3-chloro-6-[(2-oxopyrrolidin-1-yl)methyl]pyridin-2-yl}-N-{[(4-fluorophenyl)(methyl)amino]sulfonyl}-N-methylpiperidine-4-carboxamide
1-{5-butyryl-3-cyano-6-[(2-oxopyrrolidin-1-yl)methyl]pyridin-2-yl}-N-[(cyclopentylmethyl)sulfonyl]-N-methylpiperidine-4-carboxamide
1-{3-chloro-6-[(2-oxopyrrolidin-1-yl)methyl]-5-pentanoylpyridin-2-yl}-N-[(2,4-difluorobenzyl)sulfonyl]-N-methylpiperidine-4-carboxamide, and
N-(benzylsulfonyl)-1-{5-butyryl-3-fluoro-6-[(2-oxopyrrolidin-1-yl)methyl]pyridin-2-yl}piperidine-4-carboxamide;
or a pharmaceutically acceptable salt thereof.

20. A pharmaceutical composition comprising a compound according to claim 1 and a pharmaceutically acceptable adjuvant, diluent, and/or carrier.

21-23. (canceled)

24. A method of treatment of a platelet aggregation disorder comprising administering to a patient suffering from such a disorder a therapeutically effective amount of a compound according to claim 1.
